(19) United States
(12) Patent Application Publication
(10) Pub. No.: US 2010/0234377 A1
(43) Pub. Date: Sep. 16, 2010

(54) PIPERAZINE COMPOUNDS FOR THE INHIBITION OF HAEMATOPOIETIC PROSTAGLANDIN D SYNTHASE

(75) Inventors: Babette Aicher, Frankfurt (DE); Arndt-Rene Kelter, Alfter (DE); Thomas Stephen Coulter, Wantage (GB); Steven Taylor, Didcot (GB); Adams James Davenport, Didcot (GB); Thomas Hesterkamp, Hamburg (DE); Christian Kirchhoff, Tornesch (DE)

Correspondence Address: DRINKER BIDDLE & REATH (DC), 1500 K STREET, N.W., SUITE 1100, WASHINGTON, DC, 20005-1209, US

(73) Assignee: Evotec AG, Hamburg (DE)

(21) Appl. No.: 12/594,789

(22) PCT Filed: Apr. 7, 2008
(86) PCT No.: PCT/GB08/01209

§ 371 Date: Apr. 13, 2010

(30) Foreign Application Priority Data
Apr. 5, 2007 (GB) 0706793.7

(51) Int. Cl. A61K 31/5377; C07D 401/06; A61K 31/497; C07D 403/12; C07D 413/12; C07D 241/04; A61K 31/4965; A61P 3/00; A61P 9/00; A61P 17/02; A61P 25/00; A61P 29/00; A61P 37/08

(52) U.S. Cl. 514/235.8; 544/365; 514/253.13; 544/357; 514/252.11; 544/121; 544/390; 514/255.01

(57) ABSTRACT

The present invention relates to compounds of general formula (I):

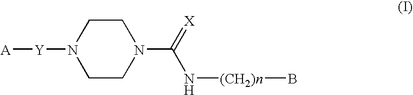

wherein A, Y, X, n and B are as defined herein;
   and their use in the treatment and prevention of metabolic disorders, inflammatory conditions, allergic conditions, fever, pain including allodynia and nociception, eating disorders, cachexia, brain injuries, cancer of the genitals, sleep apnea, cardiovascular disease, flush effect associated with nicotinic acid and related compounds or for the promotion of wound healing. Certain compounds of general formula (I) are new and the invention also relates to these compounds and to their use in medicine.

The present invention relates to the use of piperazine compounds for the production of pharmaceutical compositions for the prophylaxis and/or treatment of diseases that can be influenced by the inhibition of haematopoetic prostaglandin D Synthase (also referred to as GST2). More especially, the invention relates to the treatment or prevention of metabolic disorders, in particular obesity and other diseases of the lipid and carbohydrate metabolism, the complications associated with such disorders, inflammation, allergic conditions and cardiovascular disease. Some of the compounds are new and the invention also relates to these novel compounds, to their medical use and to pharmaceutical compositions containing them.

The development of obesity is based on an imbalance between nutrient uptake and nutrient utilisation, where excess energy is stored in the form of fat rather than being used. The primary cell type storing fat is the adipocyte. In the face of excess nutrient, the process of adipogenesis is triggered. Adipogenesis comprises the generation of new lipid-filled adipocytes from mesenchymal precursor cells as well as the size enlargement of existing adipocytes.

Adipogenesis and the maintenance of the adipocyte phenotype is controlled by a complex cascade of transcription factors, most notably by members of the peroxisome proliferators-activated receptors (PPAR). These factors control the expression of adipocyte-specific genes, such as the fatty acid binding protein aP2, the hormone leptin, and many others. PPARs belong to the nuclear receptor superfamily of ligand activated transcription factors. Once activated, PPARs heterodimerise with 9-cis retinoic acid receptors (RXR) and bind to specific DNA response elements to activate gene transcription.

The PPAR family consists to date of 3 members, PPARalpha, beta/delta, and gamma. They differ in their expression patterns, their specific ligands and the target genes being regulated. The PPARgamma isoform is specifically expressed in the adipose tissue where activation of PPARgamma activates the differentiation of pre-adipocytes into mature adipocytes (Endocrine Rev., 20, 649-688, 1999). Animal models show that total deficiency in PPARgamma leads to lipodystrophy, a state wherein no adipocytes can be formed and maintained (Mol. Cell, 4, 585-595, 1999). Thus, in a general sense, PPARgamma is viewed as the master regulator of adipogenesis. Inhibiting the process of adipogenesis by reducing the activity of PPARgamma should have efficacy against obesity and obesity-related disorders.

Accordingly, mice that are genetically haploinsufficient (i.e. lack one copy of the PPARgamma gene) are lean and resistant to development of obesity even when fed a high-fat containing diet (Mol. Cell, 4, 597-609, 1999). Similar phenotypes are observed when PPARgamma activity is reduced pharmacologically in mice (Mol. Endocrinol., 16, 2628-2644, 2002). In humans, a hypomorphic polymorphism within the PPARgamma gene leads to a reduced activity of the resulting PPARgamma protein. Individuals carrying this allele in general have a low body mass index (BMI) and less frequently develop diabetes mellitus type 2 (Nat. Genet., 26(1), 76-80, 2000). In contrast, the thiazolidinedione (TZD) class of anti-diabetic drugs were shown to activate PPARgamma to an extent beyond physiological activity. Among the side-effects of TZD treatment is a profound weight gain, in line with the pivotal role of PPAR gamma.

The naturally occurring prostaglandin 15-deoxy-Δ^{12,14}PGJ2 has been shown to be a potent ligand for PPARgamma and is able to drive fat cell differentiation in vitro (Cell, 83(5), 803-812, 1995). 15-deoxy-Δ^{12,14}PGJ2 is generated from arachidonic acid in a multistep process involving both enzymatic and non-enzymatic steps. The last enzymatic step within this cascade is the generation of prostaglandin D2 (PGD2) from prostaglandin H2 (PGH2). PGD2 is then non-enzymatically converted into the so-called J-series of prostaglandins, ultimately leading to 15-deoxy-Δ^{12,14}PGJ2.

One of the enzymes catalysing the conversion of PGH2 into PGD2 is the haematopoietic prostaglandin D Synthase (H-PGDS, also known as GST2). GST2 is expressed in the adipose tissue, and its expression is strongly up-regulated in obesity (see WO 03/040296). Therefore, inhibition of GST2 activity represents a novel therapeutic principle to treat metabolic disorders via reduction of PPARgamma activity. Therapeutic effects resulting from inhibition of GST2 via alternative pathways are possible.

Obesity is associated with an increased risk of associated diseases such as cardiovascular diseases, hypertension, diabetes, hyperlipidemia and an increased mortality. Diabetes (insulin resistance) and obesity are part of the “metabolic syndrome” which is defined as the linkage between several diseases (also referred to as syndrome X, insulin-resistance syndrome, or deadly quartet). It has been suggested that the control of lipid levels and glucose levels is required to treat diabetes type II, heart disease, and other occurrences of metabolic syndrome (see e.g., Diabetes, 48, 1836-1841, 1999; and JAMA, 288, 2209-2716, 2002).

Metabolic diseases of the carbohydrate metabolism include impaired glucose tolerance and diabetes mellitus, which is defined as a chronic hyperglycemia associated with resulting damages to organs and dysfunctions of metabolic processes. Depending on its etiology, one differentiates between several forms of diabetes, which are either due to an absolute (lacking or decreased insulin secretion) or to a relative lack of insulin. Diabetes mellitus Type I (IDDM, insulin-dependent diabetes mellitus) is of auto-immune etiology, leading to an insulitis with the subsequent destruction of the beta cells of the islets of Langerhans which are responsible for the insulin synthesis. In addition, in latent autoimmune diabetes in adults (LADA; Diabetes Care. 8, 1460-1467, 2001) beta cells are being destroyed due to autoimmune attack resulting in elevated blood glucose levels (hyperglycemia).

Diabetes mellitus Type II is associated with a resistance to insulin in the liver and the skeletal muscles, but also with a defect of the islets of Langerhans. High blood glucose levels (and also high blood lipid levels) in turn lead to an impairment of beta cell function and to an increase in beta cell apoptosis.

Diabetes is a very disabling disease, because today's common anti-diabetic drugs do not control blood sugar levels well enough to completely prevent the occurrence of high and low blood sugar levels. Out of range blood sugar levels are toxic and cause long-term complications for example retinopathy, renopathy, neuropathy and peripheral vascular disease. There is also a host of related conditions for which persons with diabetes are substantially at risk.

Lipid disorders cover a group of conditions which cause abnormalities in the level and metabolism of plasma lipids and lipoproteins. Thus, hyperlipidemias are of particular clinical relevance since they constitute an important risk factor for the development of atherosclerosis and subsequent vascular diseases such as coronary heart disease. Several complications and secondary disorders are associated with lipid disorders.

A well-known physiological condition associated with obesity is a chronic low-grade inflammation of the adipose tissue. A hallmark of this inflammatory condition is the increased production of pro-inflammatory cytokines, such as tumour necrosis factor alpha or interleukin 6 in the adipose tissue of obese subjects. Adipose inflammation also manifests in the increased infiltration of the fat tissue by activated macrophages, which are believed to sustain the inflammatory environment. Many of the major co-morbidities of obesity, such as insulin resistance, are known to be aggravated by the systemic inflammatory condition.

Prostaglandins are key mediators of inflammation. The most widely used class of anti-inflammatory and analgetic substances, the so-called non-steroidal anti-inflammatory drugs (NSAIDs), mediates its effects by blocking the conversion of arachidonic acid to the common precursor PGH2, which is catalysed by the cycloxygenase (COX) enzymes. Though widely used, NSAIDs exhibit many adverse effects, the most serious being gastric damage. It is estimated that worldwide NSAID use leads to a large number of deaths per year caused by severe gastric bleeding (N. Engl. J. Med., 340(24), 1888-1899, 1999). It is currently believed that these adverse effects are due to the inhibiton of prostaglandin E2 (PGE2) synthesis in the gastric mucosa.

PGD2 and its derivatives are well known to affect inflammatory conditions. For example, in animal models of allergic airway inflammation, it has been shown that the deletion of the PGD2 receptor results in a dramatic decrease of leukocyte infiltration into the lung. PGD2 has been shown to exert potent chemotactic effects on a variety of immune cells (e.g. granulocytes, Th2 cells, macrophages) implicated in inflammation.

PGD2 and its derivatives are well known to affect inflammatory conditions. PGD2 is the major eicosanoid produced by activated mast cells in situations of allergic inflammation and is also produced by various antigen presenting cells (J. Immunol., 143, 2982-2989, 1989) and monocytes/macrophages. In addition, PGD2 potently induces bronchoconstriction (N. Engl. J. Med., 311, 209-213, 1984), mucous protection from lung epithelia and vasodilation (J. Clin. Invest., 67, 1695-1702, 1981). Furthermore, more recent data highlight a role for PGD2 in mediating leukocyte infiltration into the asthmatic lung. For example, in animal models of allergic airway inflammation, it has been shown that the deletion of the PGD2 receptor results in a dramatic decrease of leukocyte in bronchoalveolar lavage fluid (Science, 287, 2013-2017, 2000). Correspondingly, increased PGD2 levels and therefore increased allergic lung inflammation could be observed in a transgenic animal model overexpressing the human form of GST2 (J. Immunol., 168, 443-449, 2002). By activating a newly discovered second receptor (chemoattractant-related exepressed on Th2 cells, J. Exp. Med., 193, 255-261, 2001), PGD2 has been shown to exert potent chemotactic effects on a variety of immune cells (e.g., granulocytes, Th2 cells, macrophages) implicated in inflammation (J. Exp. Med. 193, 255-261, 2001). Another class of arachidonic acid-derived eicosanoids, the leukotrienes, are also implicated in mediating aspects of asthmatic leukocyte infiltration. The Lukast family of marketed anti-asthmatic drugs antagonises leukotriene receptors, thereby ameliorating asthmatic symptoms (Expert Opin. Pharmacother., 5(3), 679-686, 2004).

Therefore, reduction of PGD2 production by inhibition of GST2 activity represents a novel therapeutic principle for treating inflammatory and allergic disorders such as asthma.

Since production of PGD2 is catalysed by GST2, inhibitors of GST2 are likely to be useful in the treatment of inflammation and allergic conditions, both as an alternative to NSAIDs, for example in the treatment of conditions such as rheumatoid arthritis, osteoarthritis and psoriatic arthritis, and in the treatment of conditions in which COX inhibitors such as NSAIDs are contra indicated. Examples of such inflammatory conditions include granuloma, systemic lupus erythematosus, inflammatory bowel disease, Crohn's disease, ulcerative colitis, multiple sclerosis and other demylelinating diseases, systemic vasculitis, amyotrophic lateral sclerosis (ALS or Lou Gehrig's disease), hypothyroidism, chronic obstructive pulmonary disease (COPD), and psoriasis. The compounds are also useful in the promotion of wound healing and for treating brain injuries.

Examples of allergic conditions which are mediated by GST2 include anaphylaxis, allergic rhinitis (hay fever), atopic dermatitis and mastocytosis.

In addition, it appears that inhibitors of GST2 are of use in the treatment of cardiovascular conditions such as atherosclerosis, stroke and thrombosis and also a number of other conditions including fever and pain, for example allodynia and nociception.

Inhibition of GST2 activity will furthermore increase and change the steady state concentrations of the eicosanoids upstream from PGD2. The physiological effects of the numerous eicosanoids comprise counteracting principles. Therefore their amplification may potentially be of therapeutic relevance in the above mentioned disorders. Known physiological and pharmacological effects are for example the shunting of unused arachidonic acid into the 5-lipoxygenase pathway in certain situations of NSAID sensitivity (Curr. Drug Targets Inflamm. Allergy, 1(1), 1-11, 2002). It is therefore likely that other prostaglandins may be produced from unused or surplus PGH2, the precursor of PGD2, thus altering the bias of the prostanoid profile.

WO 03/040296 discloses a link between human GST2 genes, particularly the variants of the human GST2 genes, and diseases which are associated with the regulation of body weight or thermogenesis. It is postulated that human GST2 genes, particularly the GST2 variants are involved in diseases such as metabolic diseases including obesity, eating disorders, cachexia, diabetes mellitus, hypertension, coronary heart disease, hypercholesterolemia, dyslipidemia, osteoarthritis, biliary stones, cancer of the genitals and sleep apnea, and in diseases connected with the ROS defence (reactive oxygen species defence), such as e.g. diabetes mellitus and cancer.

It is known that nicotinic acid and its receptors may be useful for the development of anti-dyslipidemic drugs. However, an undesired side-effect of these compounds is a strong flush, which appears to have a prostaglandin D2 component (Z. Benyo et al, Archives of Pharmacology 2005, vol 371 Suppl 1, R9). An inhibitor of prostaglandin D2 synthase may therefore be useful in treating this effect, especially when administered in combination with nicotinic acid or a related compound.

The problem underlying the present invention is to provide potent and selective GST2 inhibitors which may effectively and safely be used for the treatment of metabolic diseases and their consecutive complications and disorders.

It has now surprisingly been found that a series of piperazine compounds are potent and selective GST2 inhibitors which may effectively and safely be used in a method for the treatment of metabolic diseases and their consecutive complications and disorders, the method comprising administering to a patient in need of such treatment an effective amount of a piperazine compound as described below.

In the present invention there is provided a compound of general formula (I):

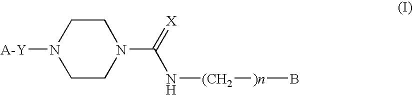

wherein

X is O or S;

Y is C═O or CR¹R²
R¹ and R² are each independently halogen or alternatively R¹ and R² may combine to form an alkylene chain —(CH₂)ₘ—, where m is 2 to 4;

A is a 5 to 10 membered aromatic or heteroaromatic ring system which may optionally be substituted with one or more substituents chosen from halogen, C₁₋₃ alkyl, C₁₋₃ haloalkyl, CN, OR³, R³, SR³, SOR³, SO₂R³, NO₂, CONH₂, CH₂OR³, CH₂NR³R⁴ or NR³R⁴;
R³ and R⁴ are each independently hydrogen, C₁₋₄ alkyl or C₁₋₄ haloalkyl;

or when there are two OR³ substituents on adjacent positions of the group A, the two R³ groups may combine to form an alkylene chain or alkenylene chain having from 1 to 3 carbon atoms and optionally substituted by one or more halogen atoms;

n is 0 or 1;

B is a 5 to 10 membered aromatic or heteroaromatic ring system optionally substituted with one or more substitutents chosen from halogen, CN, NO₂, R¹⁰, —OR¹⁰, —CO₂R¹⁰, —COR¹⁰, —CONR¹⁰R¹¹, —NR¹⁰COR¹¹, NR¹⁰CO₂R¹¹, —SO₂NR¹⁰R¹¹, —SONR¹⁰R¹¹, —SOR¹⁰, —SO₂R¹⁰, —NR¹⁰SO₂NR¹¹R¹², —SR¹⁰, —NR¹⁰R¹¹, —OCOR¹⁰, —NR¹⁰SO₂R¹¹, —NR¹⁰SOR¹¹, —N(SO₂R¹⁰)₂, —NR¹⁰(CH₂)_{q}CO₂R¹¹, or —O—(CH₂)_{q}T;
R¹⁰, R¹¹ and R¹² are each independently H, or C₁₋₆ alkyl, C₁₋₆ haloalkyl, or a group T;
   wherein T is a C₃₋₇ cycloalkyl, C₃₋₇ heterocyclyl, —C₁₋₆ alkyl (C₃₋₇ cycloalkyl), —C₁₋₆ alkyl(C₃₋₇ heterocyclyl), C₅₋₁₀ aromatic or C₅₋₁₀ heteroaromatic group, any of which is optionally substituted with one or more substituents chosen from C₁₋₆ alkyl, C₁₋₆ haloalkyl, O—C₁₋₆ alkyl, O—C₁₋₆ haloalkyl, halogen, CN, NO₂, R³, —CO₂R³, —COR^{S}, —CONR³R⁴, —NR³COR¹¹, —NR³CO₂R⁴, —SO₂NR³R⁴, —SONR³R⁴, —SOR³, —SO₂R³, —NR³SO₂NR⁴R⁵, —SR³, —NR³R⁴, —OCOR³, —NR³SO₂R⁴, —NR³OR⁴S, —N(SO₂R³)₂ or —NR³(CH₂)_{q}CO₂R⁴;
      wherein R³ and R⁴ are as defined above and R⁵ is as defined for R³ and R⁴;
or when there are two OR¹⁰ substituents on adjacent positions of the group B, the two R¹⁰ groups may combine to form an alkylene chain or alkenylene chain having from 1 to 3 carbon atoms;
q is an integer of 1 to 6

or a pharmacologically acceptable salt, hydrate, solvate, complex, polymorph or prodrug thereof;

provided that:
B is not benzisoxazole;
when A is phenyl or pyridyl, B is not phenyl substituted with a pyrrolidinyl or piperidinyl groupdisubstituted with a methoxy and a piperazinyl group;
when A is furyl, B is not phenyl substituted with halogen or —CF₃;
when A is unsubstituted phenyl, B is not unsubstituted phenyl or phenyl substituted with trifluoromethyl.

In some cases, it is also preferred that in compounds of general formula (I):
when A is phenyl or pyridyl, B is not pyrazole substituted with phenyl or substituted phenyl;
when A is phenyl substituted by OH or methoxy, B is not phenyl substituted by trifluoromethyl;
when A is unsubstituted phenyl, B is not phenyl substituted with C(O)O—C₁₋₆ alkyl.

Compounds of general formula (I) are potent and selective GST2 inhibitors and are therefore useful as pharmaceutical agents especially for the treatment or prevention of metabolic disorders, inflammatory conditions, allergic conditions, fever, pain including allodynia and nociception, eating disorders, cachexia, brain injuries, cancer of the genitals, sleep apnoea, cardiovascular disease, flush effect associated with nicotinic acid and related compounds or for the promotion of wound healing.

Compounds similar to those of general formula (I) are known. For example, WO 98/37077 and WO 99/42107 both relate to a series of compounds which are said to have activity as calcitoninin mimetics and to be useful in the inhibition of bone resorption.

WO 96/21648 discloses antitumour agents which are piperazine derivatives linked to a pyridine ring system.

WO 99/16751 relates to inhibitors of coagulation factor Xa which can be used for treating or preventing thromboembolic disorders.

U.S. Pat. No. 4,329,344 and U.S. Pat. No. 4,223,034 also relate in general to compounds which have structures falling within general formula (I), although no piperazines are actually exemplified. These compounds are said to have tranquilizing activity.

Verderame and colleagues disclose piperazine compounds which are said to have possible activity as sedatives or other psychologically active compounds (see, J. Med. Chem., 11(5), 1090-1092 (1968)).

Nagarajan et al, Indian Journal of Chemistry, 24B, 934-939 (1985) also discloses piperazine derivatives. These compounds are said to have anticonvulsant properties.

WO 97/28141 relates to 5HT_{1D} antagonists of similar, but not identical, structure to that of general formula (I). These compounds are said to be of use in the treatment of depression, obsessive compulsive disorder, anxiety, panic attacks, schizophrenia, aggressiveness, bulaemia (bulimia), alcoholism and neurodegenerative disorders such as Parkinson's disease and Alzheimer's disease.

WO 02/059098 relates to compounds which are activators of human peroxisome proliferators activated receptors (PPARs) and are said to be of use in the treatment of conditions such as diabetes, hyperlipidemia and obesity. The document discloses compounds comprising a piperazine ring linked to a CONH-aryl group. However, the 4-position of the piperazine ring is linked via a methylene group to a 5-membered aromatic ring system which, in turn is disubstituted with a phenyl group and a further bulky group having aromatic characteristics. The present inventors have surprisingly found, however that it is not optimal to substitute the piperazine ring system with such a bulky group and that much better activity is obtained when the size of the moiety “A” in general formula (I) is limited. Furthermore, the compounds of general formula (I) do not bind to PPARy.

JP 2001 199901 relates to compounds which can bind to the light chain of myosin and are intended for use in the treatment of diabetes and obesity. The compounds are similar, although not the same, as some of the compounds of the present application but are believed to be too sterically bulky to be of pharmaceutical use, as according to Lipinski's rules they have excessive molecular weight (Adv. Drug Deliv. Rev., 23, 3-25, (1997)).

WO 99/07672 relates to compounds which are similar to the compounds of general formula (I). These compounds are said to be modulators of K_{ATP} channels and therefore to be useful in the treatment of many of the same conditions as the compounds of the present invention. However, there is no evidence presented in the document that the compounds do, in fact, have the claimed activity. The present inventors have tested a large number of compounds of formula (I) and have found that even those which are most similar to the compounds of WO 99/07672 do not have K_{ATP} channel modulating activity. They then went on to test several comparator compounds which fall outside the scope of general formula (I), but which were exemplified in WO 99/07672 and have found that these do not have K_{ATP} channel modulating activity either. In view of these results, it would certainly not have been expected from the teaching of WO 99/07672 that the compounds of general formula (I) would have been effective in the treatment of diseases and conditions such as asthma, diabetes, obesity and the other conditions mentioned above because they are not K_{ATP} channel modulators.

WO 2004/072025 relates to compounds similar to those of general formula (I) in which the group B is a 6-membered aromatic ring substituted with one of the following:

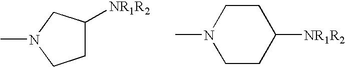

EP 1437344 relates to compounds similar to those of general formula (I) but in which the group equivalent to group B of general formula (I) is substituted with OCHPh₂. These compounds are said to be useful in the treatment of inflammatory digestive system disease, irritable bowel syndrome, allergic rhinitis, hypercholesterolaemia and arterial sclerosis, prevention of obesity and lowering of blood glucose.

WO 03/037274 relates to compounds similar to those of general formula (I) which are said to have sodium channel inhibiting activity.

WO 97/24328 relates to compounds which are said to be inhibitors of leukotriene synthesis and which are therefore said to have use in the treatment of conditions such as asthma, allergies and arteriosclerosis. The compounds all have a group CHPh in the equivalent position to the Y group of general formula (I).

EP 0638553 relates to compounds which are said to be of use in the treatmet of arteriosclerosis and diabetes. The compounds are similar, but not identical, to those of general formula (I).

WO2006/074025 relates to compounds which are similar to those of general formula (I) but are not identical. These compounds are said to be of use for the treatment of inflammation and diabetes.

WO2006/085108 relates to compounds which are said to be of use as anti-inflammatory agents. The compounds are similar to the compounds of general formula (I) but the group corresponding to the A group of general formula (I) is much larger than those of the compounds of the present invention.

WO2006/105670 relates to calcium channel blockers which are compounds similar to those of the present invention but in which the group equivalent to le is a phenyl. We have found that compounds of this type are less effective GST2 inhibitors than the compounds of general formula (I).

There are a variety of other piperazine compounds disclosed in the prior art which have similar, though not identical, structures to that of general formula (I) but there is no disclosure or suggestion anywhere of the use of such compounds in the treatment of metabolic disorders such as obesity and diabetes, or, indeed of any of the other conditions mentioned above.

In the present specification, the term “C₁-C₆” alkyl refers to a straight or branched chain fully saturated hydrocarbon group having from one to six carbon atoms.

Examples include methyl, ethyl, n-propyl, ⁱpropyl, n-butyl, ⁱbutyl, ^{t}butyl, n-pentyl and n-hexyl groups. Similarly, “C₁-C₄ alkyl” refers to an alkyl group having up to four carbon atoms.

“C₁-C₆ haloalkyl” refers to a C₁-C₆ alkyl group in which one or more of the hydrogen atoms are replaced by halogen atoms.

“C₁-C₆-heteroalkyl” refers to a C₁-C₆-alkyl group in which one or more of the carbon atoms is replaced by NH, O or S.

“halogen” refers to fluorine, chlorine, bromine or iodine.

“C₅-C₁₀-cycloalkyl” refers to an unsaturated or saturated cyclic hydrocarbon having from 5 to 10 ring carbon atoms.

“C₅-C₁₀-heterocyclyl” is similar to C₅-C₁₀-cycloalkyl except that one or more of the ring carbon atoms is replaced by NH, O or S.

A “five to ten membered aromatic ring” refers to an aromatic ring system having from five to ten ring carbon atoms and either a single ring or two fused rings. Examples include phenyl, naphthyl and indenyl groups.

A “five to ten membered heteroaromatic ring” system is a ring system having aromatic character in which one or more of the ring carbon atoms is replaced by NH, N, O or S. The heteroaromatic ring system may comprise a single ring or a fused ring system and in fused systems, one ring may be partially saturated. Examples include pyridine, pyrimidine, pyridazine, pyrrolyl, isoxazolyl, isothiazolyl, pyrazolyl, oxazolyl, oxadiazolyl, thiadiazolyl, thiazolyl, imidazolyl, triazolyl, tetrazolyl, furyl, triazinyl, thienyl, benzoxazolyl, benzothiazolyl, benzothiadiazolyl, dihydrobenzofuranyl, indole, indazole, isoindazole, dihydrobenzofuranyl, indolizinyl, quinolizinyl, cinnolinyl, phthalazinyl, quinazolinyl, naphthyridinyl, carbazolyl, benzodioxolyl, quinoxalinyl, purinyl, furazanyl, isobenzylfuranyl, benzimidazolyl, imidazo[1,2-a]pyridine, benzofuranyl, benzothienyl (including S-oxide), quinolyl, indolyl, isoquinolyl, dibenzofuranyl, napthyridyl.

Pharmaceutically acceptable salts of the compounds of general formula (I) can be formed with numerous organic and inorganic acids and bases. Exemplary acid addition salts including acetate, adipate, alginate, ascorbate, aspartate, benzoate, benzenesulfonate, bisulphate, borate, butyrate, citrate, camphorate, camphersulfonate, cyclopentanepropionate, digluconate, dodecyl sulphate, ethane sulfonate, fumarate, glucoheptanoate, glycerophosphate, hemisulphate, heptanoate, hexanoate, hydrochloride, hydrobromide, hydroiodide, 2-hydroxyethane sulfonate, lactate, maleate, methane sulfonate, 2-naphthalene sulfonate, nicotinate, nitrate, oxalate, pamoate, pectinate, persulphate, 3-phenyl sulfonate, 3-phenylpropionate, phosphate, picrate, pivalate, propionate, salicylate, succinate, sulphate, sulfonate, tartrate, thiocyanate, toluene sulfonate such as tosylate, undecanoate, or the like.

Basic nitrogen-containing moieties can be quaternized with such agents as lower alkyl halides, such as methyl, ethyl, propyl, and butyl chloride, bromide and iodide; dialkyl sulfates like dimethyl, diethyl, dibutyl, and diamyl sulfates, long-chain alkyl halides such as decyl, lauryl, myristyl and stearyl chloride, bromide and iodide, or aralkyl halides like benzyl and phenethyl bromides, or others. Water soluble or dispersible products are thereby obtained.

Pharmaceutically acceptable basic addition salts include but are not limited to cations based on the alkaline and alkaline earth metals such as sodium, lithium, potassium, calcium, magnesium, aluminium salts and the like, as well as non toxic ammonium quarternary ammonium, and amine cations, including but not limited to ammonium, tetramethylammonium, tetraethylammonium, methylamine, dimethylamine, trimethylamine, triethylamine, ethylamine and the like. Other representative amines useful for the formation of base addition salts include benzazethine, dicyclohexyl amine, hydrabine, N-methyl-D-glucamine, N-methyl-D-glucamide, t-butyl amine, diethylamine, ethylendiamine, ethanolamine, diethanolamine, piperazine and the like and salts with amino acids such as arginie, lysine, or the like.

In particularly active compounds of the present invention, A is naphthyl or a monocyclic aromatic or heteroaromatic ring system with 5 or 6 ring atoms. However, monocyclic aromatic or heteroaromatic groups with 6 ring atoms are preferred. Examples of particularly suitable A groups include phenyl, pyridyl or pyrazinyl, more especially phenyl or pyridyl, optionally substituted with one or more halogen, NO₂, CN, CONH₂, CH₂OH, CH₂NR³R⁴, NR³R⁴, SR³, SOR³, SO₂R³, C₁-C₃ alkyl, C₁-C₃ haloalkyl, O(C₁-C₃ alkyl) or O—(C₁-C₃ haloalkyl) groups, where R³ and R⁴ are hydrogen or methyl.

Particularly suitable A groups are unsubstituted or substituted with one or two substituents chosen from chloro, fluoro, trifluoromethyl, methoxy, ethoxy, trifluoromethoxy, difluoromethoxy, cyano and nitro.

When A is a pyridyl group, it is preferred that it is a 2- or 3-pyridyl group.

In more active compounds Y is C═O.

In suitable compounds of general formula (I), n is 0.

Suitably, X is O.

Compounds with all of these preferred features are particularly suitable for use as pharmaceuticals as they have relatively high macrosomal stability in comparison with other compounds of general formula (I).

These preferred compounds are compounds of general formula (Ia):

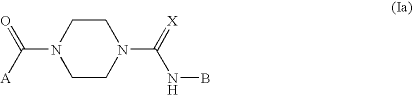

wherein A is phenyl, 2-pyridyl or 3-pyridyl optionally substituted with one or two substituents chosen from chloro, fluoro, trifluoromethyl, methoxy, ethoxy, trifluoromethoxy, difluoromethoxy, cyano and nitro; and B and X are as defined for general formula (I).

In preferred compounds of general formula (Ia), independently or in any combination:

X is O; and

A is halophenyl or 2-pyridyl substituted with halo.

Particularly suitable compounds of general formula (Ia) are those in which A is 3-halo phenyl or 6-halo pyridin-2-yl, i.e:

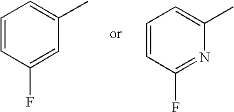

Suitably, the halo substitutent is fluoro.

It is greatly preferred that any substituents on the ring B are in a position other than that adjacent the atom which links the group B to the —NH—(CH₂)ₙ— moiety of the remainder of the molecule.

Preferred T groups include C₃₋₇ cycloalkyl, C₃₋₇ heterocyclyl, —C₁₋₆ alkyl (C₃₋₇ cycloalkyl), —C₁₋₆ alkyl (C₃₋₇ heterocyclyl), C₅₋₁₀ aromatic or C₅₋₁₀ heteroaromatic group, any of which is optionally substituted with one or more substituents chosen from C₁₋₆ alkyl, C₁₋₆ haloalkyl, O—C₁₋₆ alkyl, O—C₁₋₆ haloalkyl, halogen, CN, C(O)NH₂, C(O)NHCH₃, S(O)₂CH₃ or NO₂.

Particularly suitable compounds are those in which B is a fused 5,6- or 6,6-bicyclic aryl or heterobiaryl group, which may be partially saturated, or alternatively a monocyclic aryl or heteroaryl group having 5 or 6 ring atoms.

Particular examples of fused ring systems suitable as the group B include benzothiazole, benzimidazole, benzoxazole, naphthalene, quinoline or benzothiophene and also partially saturated systems in which B is phenyl substituted by two OR¹⁰ groups where the R¹⁰ groups together form an alkylene or alkenylene bridge having one to three carbon atoms.

When the group B is a fused 5,6-bicyclic system, it may be joined to the remainder of the molecule via the 5-membered ring. Thus 2-benzothiazole, 2-benzimidazole, 2-benzoxazole are all preferred B groups. Alternatively, however, the link may be via the 6-membered ring of a 5,6-bicyclic moiety and thus, for example, B may be a 5-benzo[b]thiophen group. Preferred partially saturated fused ring systems include benzodioxolyl, benzodioxinyl and dihydrobenzodioxinyl and other preferred fused B groups are 3-quinoline and naphthalene, especially 1-naphthalene.

When the group B is a fused ring system, it is preferred that the group B is unsubstituted or is substituted with halogen, R¹⁰, —NR¹⁰¹¹, —CONR¹⁰R¹¹, —NHCOR¹¹, —NR¹⁰SO₂R¹¹CO₂R¹⁰ or OR¹⁰, where R¹⁰ and R¹¹ are e independently hydrogen, C₁₋₄ haloalkyl, C₁₋₄ alkyl, C₃₋₆ cycloalkyl, C₃₋₇ heterocyclyl or a 5- or 6-membered aromatic or heteroaromatic ring.

More preferably, the group B is unsubstituted or is substituted with halogen, C₁₋₄ alkyl, C₁₋₄ haloalkyl, C₃₋₆ cycloalkyl, C₃₋₇ heterocyclyl or a 5- or 6-membered aromatic or heteroaromatic ring.

It is most preferred that a fused ring system B group is unsubstituted or substituted with halogen, methyl, ethyl, difluoromethyl, trifluoromethyl, cyclopropyl or pyridyl. Preferred halo groups are fluoro and chloro but especially fluoro.

As mentioned above, other preferred compounds are those in which B is a monocyclic aryl or heteroaryl group having 5 or 6 ring atoms. Examples of such groups include phenyl and pyridyl, for example 2-pyridyl or 3-pyridyl.

When B is a pyridyl group, it is preferably a 3-pyridyl group.

When B is phenyl, it is preferred that it is unsubstituted or mono-substituted at the 3- or the 4-position (but especially the 4-position) or disubstituted at the 3- and 4-positions with any of the substituents mentioned above.

For the monocyclic B groups, a larger range of substituents may advantageously be employed, including some more bulky substituents.

In preferred compounds, the monocyclic group B is unsubstituted or substituted with halo, CN, NO₂, R¹⁰, OR¹⁰, —CO₂R¹⁰, —COR¹⁰, NR¹⁰R¹¹, NR¹⁰SO₂R¹¹, NR¹⁰COR¹¹, CONR¹⁰R¹¹, N(SO₂R¹⁰)₂, SO₂R¹⁰, SO₂NR¹⁰R¹¹ or O—(CH₂)_{q}—T, wherein R¹⁰, R¹¹, T and q are as defined above.

When the group B has a halo substituent, this is preferably fluoro, chloro or bromo, more preferably fluoro or cholo and most preferably fluoro.

When the substituent for the monocyclic B group is R¹⁰ or OR¹⁰, preferred R¹⁰ groups include hydrogen, C₁₋₆ alkyl, C₁₋₆ haloalkyl, 5- or 6-membered cyclic or heterocyclic groups or 5- or 6-membered aryl or heteroaryl groups, wherein the cyclic, heterocyclic, aryl or heteroaryl groups may be further substituted with a halo, C₁₋₄ alkyl, C₁₋₄ haloalkyl, —OC₁₋₄ alkyl, —OC₁₋₄ haloalkyl, CN, C(O)NH₂, C(O)NHCH₃, SO₂CH₃ or NO₂.

Particularly preferred R¹⁰ groups when the substituent for the monocyclic B group is R¹⁰ or OR¹⁰ include hydrogen (for OR¹⁰), methyl, ethyl, isopropyl, n-propyl, n-butyl, isobutyl, t-butyl, difluoromethyl, trifluoromethyl, piperazinyl, morpholinyl, phenyl, pyridyl, triazolyl, imidazolyl, pyrazolyl, thiazolyl, oxadiazolyl and tetrazolyl, wherein the cyclic groups may be substituted as described above.

When the substituent for a monocyclic group B is —CO₂R¹⁰, —COR¹⁰, it is preferred that R¹⁰ is hydrogen, C₁₋₆ alkyl, C₁₋₆ haloalkyl, 5- or 6-membered cyclic or heterocyclic groups or 5- or 6-membered aryl or heteroaryl groups, wherein the cyclic, heterocyclic, aryl or heteroaryl groups may be further substituted with a halo, C₁₋₄ alkyl or C₁₋₄ haloalkyl group and a pyridyl group may be present as an N-oxide.

Particularly preferred R¹⁰ groups when the substituent for the monocyclic B group is CO₂R¹⁰, —COR¹⁰ include hydrogen methyl, ethyl, morpholinyl, piperidinyl, phenyl or pyridyl wherein the cyclic groups may be substituted with a halo, C₁₋₄ alkyl or C₁₋₄ haloalkyl group, especially methyl or ethyl.

When the substituent for the monocyclic B group is NR¹⁰ R¹¹, NR¹⁰SO₂R¹¹, NR¹⁰COR¹¹, NR¹⁰CO₂R¹¹ or CONR¹⁰R¹¹, it is greatly preferred that R¹⁰ is H, except for CONR¹⁰R¹¹ substituents, where C₁₋₄ alkyl or C₁₋₄ haloalkyl are also preferred.

For substituents CONR¹⁰R¹¹, in which R¹⁰ is C₁₋₄ alkyl or C₁₋₄ haloalkyl, R¹¹ will generally also be C₁₋₄ alkyl or C₁₋₄ haloalkyl and is preferably the same as R¹⁰.

Preferred R¹¹ groups when the substituent for the monocyclic B group is NR¹⁰R¹¹, NR¹⁰SO₂R¹¹, NR¹⁰COR¹¹, NR¹⁰CO₂R¹¹ or CONR¹⁰R¹¹ include C₁₋₆ alkyl, C₁₋₆ haloalkyl, 5- or 6-membered cyclic or heterocyclic groups or 5- or 6-membered aryl or heteroaryl groups, wherein the cyclic, heterocyclic, aryl or heteroaryl groups may be further substituted with a halo, C₁₋₄ alkyl or C₁₋₄ halo alkyl group and a pyridyl group may be present as an N-oxide.

Particularly preferred R¹¹ groups when the substituent for the monocyclic B group is NR¹⁰SO₂R¹¹, NR¹⁰COR¹¹ or CONR¹⁰R¹¹ include methyl, ethyl, isopropl, n-propyl, n-butyl, isobutyl, t-butyl, trifluoromethyl, cyclopropyl, tetrahydropyranyl, phenyl or pyridyl wherein the cyclic, aryl or heteroaryl groups may be substituted with a halo, C₁₋₄ alkyl or C₁₋₄ halo alkyl group, especially methyl or ethyl and a pyridyl group may be present as its N-oxide.

When the group B is substituted with N(SO₂R¹⁰)₂, it is preferred that the R¹⁰ groups are small groups, for example methyl or ethyl, preferably methyl.

When the group B has a substituent SOR¹⁰ or SO₂R¹⁰, it is preferred that R¹⁰ is C₁₋₆ alkyl, C₃₋₆ cycloalkyl or C₃₋₆ heterocyclyl. Examples of such groups include methyl, ethyl, isopropyl, cyclopentyl, morpholinyl and piperidinyl.

Where the group B has a substituent O—(CH₂)_{q}-T, q is preferably 1 or 2 and the group T is a cycloalkyl, heterocyclyl, heteroaryl or aryl ring having 5 or 6 ring atoms, for example phenyl, piperidinyl or morpholinyl.

When the group B has more than one substituent, one of the substituents will generally be halo or trifluoromethyl, more usually halo, particularly chloro or fluoro and more especially fluoro.

When the compounds of general formula (I) are for use in the treatment of diabetes, it may be preferred that the ring B is as described above, except that, when B is a monocyclic ring system substituted with R¹⁰, the R¹⁰ group is C₁₋₆ alkyl, C₁₋₆ haloalkyl, 5- or 6-membered cyclic or heterocyclic groups or 5- or 6-membered aryl or heteroaryl groups, wherein the cyclic, heterocyclic, aryl or heteroaryl groups may be further substituted with a halo, C₁₋₄ alkyl, C₁₋₄ haloalkyl or C₃₋₆ cycloalkyl, and a pyridyl group may be present as an N-oxide.

Particularly preferred groups include methyl, ethyl, isopropyl, n-propyl, n-butyl, isobutyl, t-butyl, trifluoromethyl, morpholinyl, phenyl, pyridyl, triazolyl, imidazolyl, pyrazolyl, thiazolyl, oxadiazolyl and tetrazolyl, wherein the cyclic groups may be substituted as described above.

Examples of compounds for use in the present invention are:

4-Benzoyl-piperazine-1-carbothioic acid (4-tert-butyl-phenyl)-amide (Compound 1);

4-Benzoyl-piperazine-1-carbothioic acid (4-dimethylamino-phenyl)-amide (Compound 2);

4-Benzoyl-piperazine-1-carbothioic acid (4-nitro-phenyl)-amide (Compound 3);

4-Benzoyl-piperazine-1-carbothioic acid (4-fluoro-phenyl)-amide (Compound 4);

4-Benzoyl-piperazine-1-carbothioic acid naphthalen-1-ylamide (Compound 5);

4-[(4-Benzoyl-piperazine-1-carbothioyl)-amino]-benzoic acid methyl ester (Compound 6).

4-Benzoyl-piperazine-1-carbothioic acid p-tolylamide (Compound 7);

4-(4-Fluoro-benzoyl)-piperazine-1-carbothioic acid (4-tert-butyl-phenyl)-amide (Compound 8);

4-(4-Fluoro-benzoyl)-piperazine-1-carbothioic acid (4-phenoxy-phenyl)-amide (Compound 9);

4-(4-Fluoro-benzoyl)-piperazine-1-carbothioic acid (4-dimethylamino-phenyl)-amide (Compound 10);

4-{[4-(4-Fluoro-benzoyl)-piperazine-1-carbothioyl]-amino}-benzoic acid methyl ester (Compound 11);

4-(4-Fluoro-benzoyl)-piperazine-1-carbothioic acid (4-methoxy-phenyl)-amide (compound 12);

4-(4-Fluoro-benzoyl)-piperazine-1-carbothioic acid p-tolylamide (compound 13);

4-(Pyridine-3-carbonyl)-piperazine-1-carbothioic acid (4-dimethylamino-phenyl)-amide (Compound 14);

4-(6-fluoro-pyridine-2-carbonyl)-piperazine-1-carbothioic acid (6-morpholin-4-yl-pyridin-3-yl)-amide (Compound 15);

4-(6-Fluoro-pyridine-2-carbonyl)-piperazine-1-carbothioic acid (6-morpholin-4-yl-pyridin-3-yl)-amide (Compound 16);

4-(6-Fluoro-pyridine-2-carbonyl)-piperazine-1-carbothioic acid (4-phenoxy-phenyl)-amide (Compound 17);

4-(6-Fluoro-pyridine-2-carbonyl)-piperazine-1-carbothioic acid (4-difluoromethoxy-phenyl)-amide (Compound 18);

4-(6-Fluoro-pyridine-2-carbonyl)-piperazine-1-carbothioic acid (4-trifluoromethoxy-phenyl)-amide (Compound 19);

4-(6-Fluoro-pyridine-2-carbonyl)-piperazine-1-carbothioic acid (4-trifluoromethyl-phenyl)-amide (Compound 20);

4-(6-Fluoro-pyridine-2-carbonyl)-piperazine-1-carboxylic acid (4-trifluoromethoxy-phenyl)-amide (Compound 21);

4-(6-Fluoro-pyridine-2-carbonyl)-piperazine-1-carboxylic acid (4-trifluoromethyl-phenyl)-amide (Compound 22);

4-(3-Fluoro-benzoyl)-piperazine-1-carbothioic acid (4-trifluoromethyl-phenyl)-amide (Compound 23);

4-(3-Fluoro-benzoyl)-piperazine-1-carboxylic acid (2,6-dichloro-pyridin-4-yl)-amide (Compound 24);

4-(3-Fluoro-benzoyl)-piperazine-1-carboxylic acid (3-phenoxy-phenyl)-amide (Compound 25);

4-(3-Fluoro-benzoyl)-piperazine-1-carboxylic acid (4-phenoxy-phenyl)-amide (Compound 26);

4-(3-Fluoro-benzoyl)-piperazine-1-carboxylic acid benzo[b]thiophen-5-ylamide (Compound 27);

4-(3-Fluoro-benzoyl)-piperazine-1-carboxylic acid (4-thiophen-2-yl-phenyl)-amide (Compound 28);

4-(3-Fluoro-benzoyl)-piperazine-1-carboxylic acid [4-(2-methyl-thiazol-3-yl)-phenyl]-amide (Compound 29);

4-(3-Fluoro-benzoyl)-piperazine-1-carboxylic acid [3-(5-methyl-[1,2,4]oxadiazol-3-yl)-phenyl]-amide (Compound 30);

4-(3-Fluoro-benzoyl)-piperazine-1-carboxylic acid (3-morpholin-4-yl-phenyl)-amide (Compound 31);

4-(3-Fluoro-benzoyl)-piperazine-1-carboxylic acid (3-bromo-phenyl)-amide (Compound 32);

4-{[4-(3-Fluoro-benzoyl)-piperazine-1-carbonyl]-amino}-benzoic acid ethyl ester (Compound 33);

3-{[4-(3-Fluoro-benzoyl)-piperazine-1-carbonyl]-amino}-benzoic acid methyl ester (Compound 34);

4-(3-Fluoro-benzoyl)-piperazine-1-carboxylic acid (3-cyano-phenyl)-amide (Compound 35);

4-(3-Fluoro-benzoyl)-piperazine-1-carboxylic acid (3-nitro-phenyl)-amide (Compound 36);

4-(3-Fluoro-benzoyl)-piperazine-1-carboxylic acid (4-nitro-phenyl)-amide (Compound 37);

4-(3-Fluoro-benzoyl)-piperazine-1-carboxylic acid (3-chloro-4-morpholin-4-yl-phenyl)-amide (Compound 38);

4-(3-Fluoro-benzoyl)-piperazine-1-carboxylic acid (5-chloro-benzooxazol-2-yl)-amide (Compound 39);

Preparation of 4-(6-Fluoro-pyridine-2-carbonyl)-piperazine-1-carbothioic acid (3-fluoro-4-morpholin-4-yl-phenyl)-amide (Compound 40);

4-(3-Fluoro-benzoyl)-piperazine-1-carboxylic acid (3-fluoro-4-morpholin-4-yl-phenyl)-amide (Compound 41);

4-(3-Fluoro-benzoyl)-piperazine-1-carboxylic acid (4-benzoyl-phenyl)-amide (Compound 42);

4-(3-Fluoro-benzoyl)-piperazine-1-carboxylic acid (4-bromo-3-pyridin-4-yl-phenyl)-amide (Compound 43);

4-(3-Fluoro-benzoyl)-piperazine-1-carboxylic acid [6-(pyridin-3-yloxy)-pyridin-3-yl]-amide (Compound 44);

4-(3-Fluoro-benzoyl)-piperazine-1-carboxylic acid quinolin-3-ylamide (Compound 45);

4-(2-Fluoro-4-{[4-(3-fluoro-benzoyl)-piperazine-1-carbonyl]-amino}-phenyl)-piperazine-1-carboxylic acid tert-butyl ester (Compound 46);

4-(2,5-Difluoro-benzoyl)-piperazine-1-carboxylic acid (3-fluoro-4-morpholin-4-yl-phenyl)-amide (Compound 47);

4-(4-{[4-(2,5-Difluoro-benzoyl)-piperazine-1-carbonyl]amino}-2-fluoro-phenyl)-piperazine-1-carboxylic acid tert-butyl ester (Compound 48);

4-(2,5-Difluoro-benzoyl)-piperazine-1-carboxylic acid (4-nitro-phenyl)-amide (Compound 49);

4-(2-Fluoro-4-{[4-(3-fluoro-benzoyl)-piperazine-1-carbonyl]-amino}-phenyl)-piperazine-1-carboxylic acid isopropyl ester (Compound 50);

4-(2,5-Difluoro-benzoyl)-piperazine-1-carboxylic acid (3-fluoro-4-piperazin-1-yl-phenyl)-amide (Compound 51);

4-(3-fluorobenzoyl)-N-{3-fluoro-4-[4-(isopropylcarbamoyl)piperazin-1-yl]phenyl}piperazine-1-carboxamide (Compound 52);

N-{4-[4-(-butylcarbamoyl)piperazin-1-yl]-3-fluorophenyl}-4-(3-fluorobenzoyl)piperazine-1-carboxamide (Compound 53);

N-{4-[4-(cyclopentylcarbamoyl)piperazin-1-yl]-3-fluorophenyl}-4-(3-fluorobenzoyl)piperazine-1-carboxamide (Compound 54);

4-(2,5-difluorobenzoyl)-N-{3-fluoro-4-[4-(isopropylcarbamoyl)piperazin-1-yl]phenyl}piperazine-1-carboxamide (Compound 55);

4-(3-Fluoro-benzoyl)-piperazine-1-carboxylic acid {4-[4-(2,2-dimethyl-propionyl)-piperazin-1-yl]-3-fluoro-phenyl}-amide (Compound 56);

4-(3-Fluoro-benzoyl)-piperazine-1-carboxylic acid [3-fluoro-4-(4-isobutyryl-piperazin-1-yl)-phenyl]-amide (Compound 57)

4-(2,5-Difluoro-benzoyl)-piperazine-1-carboxylic acid [3-fluoro-4-(4-isobutyryl-piperazin-1-yl)-phenyl]-amide (Compound 58);

4-(2-Fluoro-4-{[4-(3-fluoro-benzoyl)-piperazine-1-carbonyl]-amino}-phenyl)-piperazine-1-carboxylic acid tetrahydro-furan-3-yl ester (Compound 59);

4-(3-Fluoro-benzoyl)-piperazine-1-carboxylic acid [3-fluoro-4-(4-methyl-piperazin-1-yl)-phenyl]-amide (Compound 60);

4-(3-Fluoro-benzoyl)-piperazine-1-carboxylic acid [4-(4-acetyl-piperazin-1-yl)-3-fluoro-phenyl]-amide (Compound 61);

4-(3-Fluoro-benzoyl)-piperazine-1-carboxylic acid (5-methyl-isoxazol-3-yl)-amide (Compound 62); 4-(3-Fluoro-benzoyl)-piperazine-1-carboxylic acid (6-chloro-pyridin-3-yl)-amide (Compound 63);

4-(3-Fluoro-benzoyl)-piperazine-1-carboxylic acid (1H-benzoimidazol-2-yl)-amide (Compound 64);

4-(3-Fluoro-benzoyl)-piperazine-1-carboxylic acid benzothiazol-2-ylamide (Compound 65);

4-(3-Fluoro-benzoyl)-piperazine-1-carboxylic acid (6-fluoro-benzothiazol-2-yl)-amide (Compound 66);

4-(3-Fluoro-benzoyl)-piperazine-1-carboxylic acid (6-trifluoromethoxy-benzothiazol-2-yl)-amide (Compound 67);

4-(3-Fluoro-benzoyl)-piperazine-1-carboxylic acid (6-bromo-benzothiazol-2-yl)-amide (Compound 68);

4-(3-Fluoro-benzoyl)-piperazine-1-carboxylic acid (4-phenyl-thiazol-2-yl)-amide (Compound 69);

4-(3-Fluoro-benzoyl)-piperazine-1-carboxylic acid (3-phenyl-[1,2,4]thiadiazol-5-yl)-amide (Compound 70);

4-(3-Fluoro-benzoyl)-piperazine-1-carboxylic acid (5-pyridin-4-yl-[1,3,4]thiadiazol-2-yl)-amide (Compound 71);

4-(3-Fluoro-benzoyl)-piperazine-1-carboxylic acid (5-phenyl-[1,3,4]oxadiazol-2-yl)-4-(3-Fluoro-benzoyl)-piperazine-1-carboxylic acid (4-pyridin-2-yl-thiazol-2-yl)-amide (Compound 73);

4-(3-Fluoro-benzoyl)-piperazine-1-carboxylic acid (4-pyridin-3-yl-thiazol-2-yl)-amide (Compound 74);

4-(3-Fluoro-benzoyl)-piperazine-1-carboxylic acid (4-pyridin-4-yl-thiazol-2-yl)-amide (Compound 75);

4-(3-Fluoro-benzoyl)-piperazine-1-carboxylic acid [3-(4-methyl-4H-[1,2,4]triazol-3-yl)-phenyl]-amide (Compound 76);

4-(3-Fluoro-benzoyl)-piperazine-1-carboxylic acid [3-(2-methyl-thiazol-4-yl)-phenyl]-amide (Compound 77);

4-(3-Fluoro-benzoyl)-piperazine-1-carboxylic acid [3-(2-methyl-pyrimidin-4-yl)-phenyl]-amide (Compound 78);

4-(3-Fluoro-benzoyl)-piperazine-1-carboxylic acid (5 ,6-dimethyl-benzothiazol-2-yl)-amide (Compound 79);

4-(3-Fluoro-benzoyl)-piperazine-1-carboxylic acid (6-methyl-benzothiazol-2-yl)-amide (Compound 80);

4-(3-Fluoro-benzoyl)-piperazine-1-carboxylic acid (6-methoxy-benzothiazol-2-yl)-amide (Compound 81);

4-(3-Fluoro-benzoyl)-piperazine-1-carboxylic acid (6-chloro-benzothiazol-2-yl)-amide (Compound 82);

2-{[4-(3-Fluoro-benzoyl)-piperazine-1-carbonyl]-amino}-benzothiazole-6-carboxylic acid ethyl ester (Compound 83);

4-(3-Fluoro-benzoyl)-piperazine-1-carboxylic acid (6-acetylamino-benzothiazol-2-yl)-amide (Compound 84);

4-(3-Fluoro-benzoyl)-piperazine-1-carboxylic acid (3-cyano-4-fluoro-phenyl)-amide (Compound 85);

4-(3-Fluoro-benzoyl)-piperazine-1-carboxylic acid (3-pyridin-4-yl-phenyl)-amide (Compound 86);

4-(3-Fluoro-benzoyl)-piperazine-1-carboxylic acid (3-pyridin-3-yl-phenyl)-amide (Compound 87);

4-(3-Fluoro-benzoyl)-piperazine-1-carboxylic acid (4-bromo-3-fluoro-phenyl)-amide (Compound 88);

4-(3-Fluoro-benzoyl)-piperazine-1-carboxylic acid [4-(1H-pyrazol-3-yl)-phenyl]-amide (Compound 89);

4-(3-Fluoro-benzoyl)-piperazine-1-carboxylic acid [4-(2-methyl-thiazol-4-yl)-phenyl]-amide (Compound 90);

4-(3-Fluoro-benzoyl)-piperazine-1-carboxylic acid [4-(morpholine-4-sulfonyl)-phenyl]-amide (Compound 91);

4-(3-Fluoro-benzoyl)-piperazine-1-carboxylic acid [3-(morpholine-4-sulfonyl)-phenyl]-amide (Compound 92);

4-(3-Fluoro-benzoyl)-piperazine-1-carboxylic acid (3-methylcarbamoyl-phenyl)-amide (Compound 93);

2-Fluoro-5-{[4-(3-fluoro-benzoyl)-piperazine-1-carbonyl]-amino}-benzoic acid methyl ester (Compound 94);

2-Fluoro-4-{[4-(3-fluoro-benzoyl)-piperazine-1-carbonyl]-amino}-benzoic acid methyl ester (Compound 95);

2-Fluoro-4-{[4-(3-fluoro-benzoyl)-piperazine-1-carbonyl]-amino}-benzoic acid (Compound 96);

4-(3-Fluoro-benzoyl)-piperazine-1-carboxylic acid [3-fluoro-4-(morpholine-4-carbonyl)-phenyl]-amide (Compound 97);

4-(3-Fluoro-benzoyl)-piperazine-1-carboxylic acid [3-(2-methyl-thiazol-4-yl)-phenyl]-amide (Compound 98);

4-(3-Fluoro-benzoyl)-piperazine-1-carboxylic acid [3-fluoro-4-(6-fluoro-pyridin-3-ylcarbamoyl)-phenyl]-amide (Compound 99);

4-{[4-(3-Fluoro-benzoyl)-piperazine-1-carbonyl]-amino}-benzoic acid (Compound 100);

4-(3-Fluoro-benzoyl)-piperazine-1-carboxylic acid [4-(morpholine-4-carbonyl)-phenyl]-amide (Compound 101);

4-(3-Fluoro-benzoyl)-piperazine-1-carboxylic acid (4-diethylcarbamoyl-phenyl)-amide (Compound 102);

4-(3-Fluoro-benzoyl)-piperazine-1-carboxylic acid [4-(6-fluoro-pyridin-3-ylcarbamoyl)-phenyl]-amide (Compound 103);

4-(3-Fluoro-benzoyl)-piperazine-1-carboxylic acid (4-carbamoyl-3-fluoro-phenyl)-amide (Compound 104);

4-(3-Fluoro-benzoyl)-piperazine-1-carboxylic acid (4-carbamoyl-phenyl)-amide (Compound 105);

4-(3-Fluoro-benzoyl)-piperazine-1-carboxylic acid (3-amino-phenyl)-amide (Compound 106);

4-(3-Fluoro-benzoyl)-piperazine-1-carboxylic acid {3-[(pyridine-2-carbonyl)-amino]-phenyl}-amide (Compound 107);

4-(3-Fluoro-benzoyl)-piperazine-1-carboxylic acid {3-[(1-oxy-pyridine-2-carbonyl)-amino]-phenyl}-amide (Compound 108);

4-(3-Fluoro-benzoyl)-piperazine-1-carboxylic acid {3-[(1-oxy-pyridine-3-carbonyl)-amino]-phenyl}-amide (Compound 109);

4-(3-Fluoro-benzoyl)-piperazine-1-carboxylic acid {3-[(1-oxy-pyridine-4-carbonyl)-amino]-phenyl}-amide (Compound 110);

4-(3-Fluoro-benzoyl)-piperazine-1-carboxylic acid {3-[(6-fluoro-pyridine-3-carbonyl)-amino]-phenyl}-amide (Compound 111);

4-(3-Fluoro-benzoyl)-piperazine-1-carboxylic acid (4-amino-phenyl)-amide (Compound 112);

4-(2,5-Difluoro-benzoyl)-piperazine-1-carboxylic acid (4-amino-phenyl)-amide (Compound 113);

4-(3-Fluoro-benzoyl)-piperazine-1-carboxylic acid {4-[(pyridine-2-carbonyl)-amino]-phenyl}-amide (Compound 114);

4-(3-Fluoro-benzoyl)-piperazine-1-carboxylic acid {4-[(1-oxy-pyridine-2-carbonyl)-amino]-phenyl}-amide (Compound 115);

4-(3-Fluoro-benzoyl)-piperazine-1-carboxylic acid {4-[(1-oxy-pyridine-3-carbonyl)-amino]-phenyl}-amide (Compound 116);

4-(3-Fluoro-benzoyl)-piperazine-1-carboxylic acid {4-[(6-fluoro-pyridine-3-carbonyl)-amino]-phenyl}-amide (Compound 117);

4-(2,5-Difluoro-benzoyl)-piperazine-1-carboxylic acid {4-[(1-oxy-pyridine-2-carbonyl)-amino]-phenyl}-amide (Compound 118);

4-(2,5-Difluoro-benzoyl)-piperazine-1-carboxylic acid {4-[(1-oxy-pyridine-3-carbonyl)-amino]-phenyl}-amide (Compound 119);

4-(3-Fluoro-benzoyl)-piperazine-1-carboxylic acid [4-(4-fluoro-benzoylamino)-phenyl]-amide (Compound 120);

4-(3-Fluoro-benzoyl)-piperazine-1-carboxylic acid (4-isobutyrylamino-phenyl)-amide (Compound 121);

4-(3-Fluoro-benzoyl)-piperazine-1-carboxylic acid {4-[(pyridine-3-carbonyl)-amino]-phenyl}-amide (Compound 122);

4-(3-Fluoro-benzoyl)-piperazine-1-carboxylic acid {4-[(pyridine-4-carbonyl)-amino]-phenyl}-amide (Compound 123);

4-(3-Fluoro-benzoyl)-piperazine-1-carboxylic acid {4-[(tetrahydro-pyran-4-carbonyl)-amino]-phenyl}-amide (Compound 124);

4-(3-Fluoro-benzoyl)-piperazine-1-carboxylic acid (4-benzenesulfonylamino-phenyl)-amide (Compound 125);

4-(3-Fluoro-benzoyl)-piperazine-1-carboxylic acid (4-methanesulfonylamino-phenyl)-amide (Compound 126);

4-(3-Fluoro-benzoyl)-piperazine-1-carboxylic acid (4-cyclopropanesulfonylamino-phenyl)-amide (Compound 127);

4-(3-Fluoro-benzoyl)-piperazine-1-carboxylic acid [4-(4-fluoro-benzenesulfonylamino)-phenyl]-amide (Compound 128);

N-{4-[bis(methylsulfonyl)amino]phenyl}-4-(3-fluorobenzoyl) piperazine-1-carboxamide (Compound 129);

4-(3-Fluoro-benzoyl)-piperazine-1-carboxylic acid [3-(4-fluoro-benzenesulfonylamino)-phenyl]-amide (Compound 130);

4-(3-Fluoro-benzoyl)-piperazine-1-carboxylic acid (3-cyclopropanesulfonylamino-phenyl)-amide (Compound 131);

4-(2,5-Difluoro-benzoyl)-piperazine-1-carboxylic acid (4-cyclopropanesulfonylamino-phenyl)-amide (Compound 132);

4-(2,5-Difluoro-benzoyl)-piperazine-1-carboxylic acid (4-methanesulfonylamino-phenyl)-amide (Compound 133);

4-(3-Fluoro-benzoyl)-piperazine-1-carboxylic acid (3-fluoro-4-nitro-phenyl)-amide (Compound 134);

4-(3-Fluoro-benzoyl)-piperazine-1-carboxylic acid [4-(1H-tetrazol-5-yl)-phenyl]-amide (Compound 135);

4-(3-Fluoro-benzoyl)-piperazine-1-carboxylic acid (6-amino-benzothiazol-2-yl)-amide (Compound 136);

4-(3-Fluoro-benzoyl)-piperazine-1-carboxylic acid (6-cyclopropanesulfonylamino-benzothiazol-2-yl)-amide (Compound 137);

2-{[4-(3-Fluoro-benzoyl)-piperazine-1-carbonyl]-amino}-benzothiazole-6-carboxylic acid (Compound 138);

4-(3-Fluoro-benzoyl)-piperazine-1-carboxylic acid [6-(morpholine-4-carbonyl)-benzothiazol-2-yl]-amide (Compound 139);

4-(3-Fluoro-benzoyl)-piperazine-1-carboxylic acid {6-[(pyridine-2-carbonyl)-amino]-benzothiazol-2-yl}-amide (Compound 140);

2-{[4-(3-Fluoro-benzoyl)-piperazine-1-carbonyl]-amino }1-benzothiazole-6-carboxylic acid amide (Compound 141);

4-(3-Fluoro-benzoyl)-piperazine-1-carboxylic acid [4-(3-isopropyl-[1,2,4]oxadiazol-5-yl)-phenyl]-amide (Compound 142);

4-(3-Fluoro-benzoyl)-piperazine-1-carboxylic acid (4-pyrazol-1-yl-phenyl)-amide (Compound 143);

4-(3-Fluoro-benzoyl)-piperazine-1-carboxylic acid (4-imidazol-1-yl-phenyl)-amide (Compound 144);

4-(3-Fluoro-benzoyl)-piperazine-1-carboxylic acid (4-[1,2,4]triazol-1-yl-phenyl)-amide (Compound 145);

4-(3-Fluoro-benzoyl)-piperazine-1-carboxylic acid [3-fluoro-4-(4-pyridin-2-yl-piperazin-1-yl)-phenyl]-amide (Compound 146);

4-(3-Fluoro-benzoyl)-piperazine-1-carboxylic acid (3-fluoro-4-pyridin-3-yl-phenyl)-amide (Compound 147);

4-(2,5-Difluoro-benzoyl)-piperazine-1-carboxylic acid (4-trifluoromethyl-phenyl)-amide (Compound 148);

4-(3-Fluoro-benzoyl)-piperazine-1-carboxylic acid (4-trifluoromethyl-phenyl)-amide (Compound 149);

4-(2,5-Difluoro-benzoyl)-piperazine-1-carbothioic acid (6-morpholin-4-yl-pyridin-3-yl)-amide (Compound 150);

4-(3-Fluoro-benzoyl)-piperazine-1-carbothioic acid (6-morpholin-4-yl-pyridin-3-yl)-amide (Compound 151);

4-(2-Methyl-benzoyl)-piperazine-1-carbothioic acid (6-morpholin-4-yl-pyridin-3-yl)-amide (Compound 152);

4-(3-Nitro-benzoyl)-piperazine-1-carbothioic acid (6-morpholin-4-yl-pyridin-3-yl)-amide (Compound 153);

4-(3-Fluoro-benzoyl)-piperazine-1-carboxylic acid (6-morpholin-4-yl-pyridin-3-yl)-amide (Compound 154);

4-(3-Fluoro-benzoyl)-piperazine-1-carboxylic acid (4-fluoro-phenyl)-amide (Compound 155);

4-(2-Methyl-benzoyl)-piperazine-1-carboxylic acid (4-fluoro-phenyl)-amide (Compound 156);

4-(3-Carbamoyl-benzoyl)-piperazine-1-carboxylic acid (4-fluoro-phenyl)-amide (Compound 157);

4-(3-Fluoro-benzoyl)-piperazine-1-carboxylic acid (3-carbamoyl-phenyl)-amide (Compound 158);

4-(3-Fluoro-benzoyl)-piperazine-1-carboxylic acid [3-(2H-[1,2,4]triazol-3-yl)-phenyl]-amide (Compound 159);

4-(3-Fluoro-benzoyl)-piperazine-1-carboxylic acid (3-carbamoyl-4-fluoro-phenyl)-amide (Compound 160);

4-(3-Fluoro-benzoyl)-piperazine-1-carboxylic acid [4-fluoro-3-(2H-[1,2,4]triazol-3-yl)-phenyl]-amide (Compound 161);

4-(3-Fluoro-benzoyl)-piperazine-1-carboxylic acid [3-(3-isopropyl-[1,2,4]oxadiazol-5-yl)-phenyl]-amide (Compound 162);

4-(3-Fluoro-benzoyl)-piperazine-1-carboxylic acid [4-fluoro-3-(3-isopropyl-[1,2,4]oxadiazol-5-yl)-phenyl]amide (Compound 163);

4-Benzoyl-piperazine-1-carboxylic acid (4-trifluoromethyl-phenyl)-amide (Compound 164);

4-(3-Chloro-benzoyl)-piperazine-1-carboxylic acid (4-trifluoromethyl-phenyl)-amide (Compound 165);

4-(3-Fluoro-benzoyl)-piperazine-1-carboxylic acid (3-phenylamino-phenyl)-amide (Compound 166);

4-(3-Fluoro-benzoyl)-piperazine-1-carboxylic acid [3-(4-fluoro-phenylamino)-phenyl]-amide (Compound 167);

or a pharmacologically acceptable salt, hydrate, solvate, complex, polymorph or prodrug thereof.

Compounds of general formula (I) may be prepared from compounds of general formula (II).

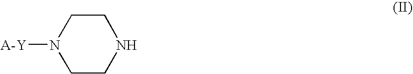

wherein A and Y are as defined in general formula (I) by reaction with a compound of general formula (III):

B—(CH₂)ₙ—N═C═X (III)

wherein B and X are as defined in general formula (I).

The reaction may be carried out in an appropriate aprotic organic solvent such as dichloromethane at a temperature of from about 0 to 50° C., usually at room temperature. The compound of general formula (II) may be immobilised on a solid support (for example a resin), which can be removed from the reaction mixture after the reaction by any known method, for example by filtration.

Compounds of general formula (III) are readily available or may be prepared by methods well known to those skilled in the art.

Compounds of general formula (II) may be prepared by the reaction of a compound of general formula (IV):

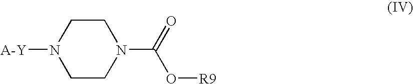

wherein A and Y are as defined for general formula (I) and R⁹ is C₁-C₆ alkyl;

with an acid, which may be in an organic solvent. The reaction may be conducted at a temperature of from about 0 to 50° C. but will generally be carried out at room temperature. The resultant organic salts of compounds of general formula (II) may be treated with carbonate resin to generate the corresponding free amine. The compound of general formula (IV) may be immobilised on a solid phase such as a resin, and the resin can be removed after the reaction by filtration.

A method for the preparation of compounds of general formula (IV) in which Y is CR¹R² is by the reaction of a compound of general formula (VII):

A-CR¹R²—Z (VII)

wherein A, R¹ and R² are as defined for general formula (I) and Z is halogen, especially bromine;

with a piperazine derivative of general formula (VI):

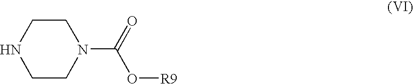

wherein R⁹ is as defined for general formula (IV).

The reaction may be conducted in an appropriate organic solvent in the presence of a base such as triethylamine.

Compounds of general formula (VI) are readily available or can be prepared by methods well known to those skilled in the art. In the compounds of general formula (VI), the R⁹ group will often be a t-butyl group since t-butoxycarbonyl (boc) is a well known amine protecting group.

Compounds of general formula (VII) are also readily available or alternatively may be prepared from compounds of general formula (VIII):

A-CHR¹R² (VIII)

wherein A is as defined for general formula (I);

by halogenation with any known halogenating agent, such as N-bromosuccinimide.

Compounds of general formula (IV) in which Y is C=O may be prepared from compounds of general formula (IX):

A-C(O)Cl (IX)

wherein A is as defined for general formula (I);

by reaction with a compound of general formula (VI) as defined above.

The reaction may be carried out in an appropriate organic solvent such as dichloromethane and the reaction mixture is preferably cooled, for example to between −5 and 5° C. during the addition of the acid chloride of general formula (IX).

Compounds of general formula (IX) are readily available or may be prepared by methods which are well known to those of skill in the art.

Compounds of general formula (IV) in which Y is C═O may also be prepared by the reaction of a compound of general formula (VI) as defined above, with a carboxylic acid of general formula (X):

A-C(O)OH (X)

wherein A is as defined above for general formula (I);

in the presence of known amide coupling reagents such as (1-(3-dimethylaminopropyl)-3-ethyl dicarbodiimide hydrochloride (EDC) and HOBt.

The reaction may be carried out in an appropriate organic solvent such as dichloromethane and the reaction mixture is preferably cooled during activation of the carboxylic acid of general formula (X), for example to between −5 and 5° C.

Compounds of general formula (X) are readily available or may be prepared by methods which are well known to those of skill in the art.

In an alternative synthetic method, compounds of general formula (I) in which Y is C═O may be formed by the reaction of a compound of general formula (XIX):

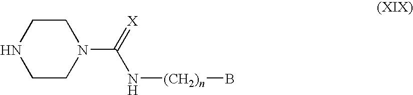

wherein X, n and B are as defined in general formula (I);

with a compound of general formula (IX) or (X) as defined above

The reaction will generally be carried out at a temperature of from about −5 to 30° C., more usually from 0 to 25° C.

Compounds of general formula (XIX) may be prepared by first reacting a compound of general formula (VI) as defined above with a compound of general formula (III) as defined above, followed by deprotection of the carbamate group and de-salting using standard conditions. The reaction may be carried out in an aprotic organic solvent such as dichloromethane at a temperature of from about 15 to 50° C., usually at room temperature. The compound of general formula (XIX) may be immobilised on a solid support (for example a resin), which can be removed from the reaction mixture after the reaction by any known method, for example by filtration.

Alternatively, compounds of general formula (XIX) can be prepared from compounds of general formula (XI):

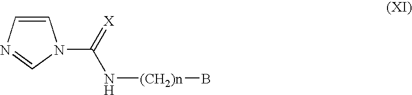

wherein B, n and X are as defined for general formula (I).

by reaction with a compound of formula (VI) as defined above. The reaction may be carried out in a polar organic solvent such as dichloromethane and at a temperature of 15 to 50° C., typically at room temperature.

The intermediate of general formula (XI) may be prepared by the reaction of a compound of general formula (XII):

B—(CH₂)ₙ—NH₂ (XII)

wherein B is as defined in general formula (I);

with di-imidazol-1-ylmethanone for compounds in which X is O or di-imidazol-1-ylmethanethione for compounds in which X is S.

The reaction may be carried out in an appropriate organic solvent such as dichloromethane at a temperature of from 15 to 50° C., but more usually at room temperature and the intermediate of general formula (XI) can be reacted in situ without purification with the compound of general formula (II).

Compounds of general formula (XII) are readily available or may be prepared by methods which are well known to those of skill in the art.

Typically a compound of general formula (XII) may be prepared from a compound of general formula (XV):

B—(CH₂)ₙ—NO₂ (XV)

where B and n are as defined above; typically by reduction for example using hydrogenation over a palladium/carbon catalyst. For some compounds, particularly those in which B is substituted with a heteroaromatic group, another reducing method may be preferred, for example by treating with tin (II) chloride.

Additional methods for the preparation of specific compounds of general formulae (XII) and (XV) are discussed in more detail below.

A further method for the preparation of compounds of general formula (I) is by the reaction of a compound of general formula (XII) as defined above with a chloroformate followed by a compound of general formula (II) as defined above. Usually, the chloroformate is an aryl chloroformate in which the aryl group has electron withdrawing substituents and a particularly suitable chloroformate compound is 4-nitrophenylchloroformate. This method is illustrated in Route 13 in the Examples below.

This route for preparing compounds of general formula (I) is very flexible as the first step, the reaction with the chloroformate can be carried out over a wide temperature range, which makes it suitable for use in preparing compounds with unreactive B groups where the reaction mixture containing the compound of general formula (XII) requires heating and also for compounds with highly reactive B groups, where the amine of general formula (XII) can be cooled. Thus, the first step of the reaction can be carried out at temperatures between −5 and 80° C., depending upon the nature of the group B. The solvent will usually be a polar organic solvent such as dichloromethane but it may be necessary to adjust the solvent if very high or very low reaction temperatures are required.

Once the first stage of the reaction is complete, excess chloroformate may be removed from the reaction mixture and the compound of general formula (II) added in the presence of a weak base. Again, suitable solvents are typically polar organic solvents such as dichloromethane and the reaction may be carried out at a temperature between 5 and 40° C., but typically at room temperature.

Yet another method for the preparation of compounds of general formula (I) is by the reaction of a compound of general formula (XXX):

B—C(═O)OH (XXX)

wherein B is as defined in general formula (I) with a stable azide-transfer agent such as diphenylphosphoryl azide (DPPA) in the presence of a suitable amine such as NEt₃, to form the corresponding acyl azide intermediate, followed by reaction with a compound of general formula (II) as defined above.

As described above, compounds of general formula (XV) are useful precursors to compounds of general formula (XII). Some compounds of formula (XV) are readily available and some can be synthesised using the methods described below. The particular method selected will depend upon the group B in the compound of general formula (XV).

For example, a compound of general formula (XVa):

O₂N—(CH₂)ₙ—B¹—OT (XVa)

where B¹ is a 5 to 10 membered aromatic or heteroaromatic ring system and T is as defined above;

may be prepared by reacting a compound of general formula (XIII):

F—B¹—(CH₂)ₙ—NO₂ (XIII)

with a compound of general formula (XIV):

T-OH (XIV)

where T is as defined for general formula (I).

The reaction may be carried out in the presence of a strong base such as sodium hydride.

Compounds of general formula (XVb):

NO₂—(CH₂)ₙ—B¹—O(CH₂)_{q}T (XVb)

may be prepared by reacting a compound of general formula (XVI):

NO₂—(CH₂)ₙ—B¹—O(CH₂)_{q}X (XVI)

where B¹ and q are as defined above and X is a leaving group, especially a halogen such as chlorine;

with a compound of general formula (XVII):

T-H (XVII)

where T is as defined above but is especially a nitrogen-containing heterocyclic ring in which the H is bound to the nitrogen atom.

Compounds of Formula (XVc):

NO₂—(CH₂)ₙ—B¹-T (XVc)

wherein B¹ is a 5 to 10 membered aromatic or heteroaromatic ring and T is a nitrogen containing heterocyclic ring such as piperazine or morpholine which is joined to the ring B¹ via the nitrogen atom;

may be prepared by reacting a compound of formula (XIII) as defined above with a compound of general formula (XVII) as defined above. This reaction is illustrated in Routes 10, 16 and 34 of the Examples below.

Compounds of Formula (XVd):

NO₂—(CH₂)ₙ—B¹—NH-T (XVd)

wherein B¹ and T are as defined above but wherein T is especially a heterocyclic or heteroaromatic group joined to the NH moiety via a carbon atom may be prepared by the reaction of a compound of general formula (XIII) as defined above with a compound of general formula (XXXI):

T-NH₂ (XXXI)

where T is a heterocyclic or heteroaromatic group joined to the NH₂ moiety via carbon atom. The reaction may be carried out in a polar solvent under mildly basic conditions.

Certain compounds of general formula (I) can be prepared from other compounds of general formula (I). For example, compounds of general formula (I) in which B is substituted by a primary amino group can be prepared by reacting a compound of general formula (I) in which B is substituted by NC(O)O(C₁-C₆ alkyl) with an acid. One example is trifluoroacetic acid, and reaction with this may be followed by treatment with a base, for example carbonate resin. Alternatively, hydrochloric acid may be used, for example in a sealed tube (See Route 23 of the Examples below).

Compounds of general formula (I) in which B is substituted with a primary amino group or a secondary amino group NHR¹⁰ may be converted to compounds of general formula (I) in which B is substituted with a group NR¹⁰C(═O)R¹¹ by reaction with a carboxylic acid of general formula (XXXII):

R¹¹—C(═O)M (XXXII)

wherein R¹¹ is as defined above and M is OH or Cl. The reaction may be carried out as shown in Route 18 or Route 19 of the Examples.

Compounds of general formula (I) in which B is substituted by a primary amino group can be converted to compounds of general formula (I) in which B is substituted with NHSO₂NR¹¹R¹² by reaction with Cl—SO₂—N═C═O and 1-chloro-ethan-2-ol. The resulting intermediate can then be reacted with a compound of general formula (XXIV):

R¹¹R¹²NH (XXIV)

wherein where R¹¹ and R¹² are as defined above.

Compounds of general formula (I) in which B is substituted with a primary amino group can also be converted to compounds of general formula (I) in which B is substituted with NHSO₂R¹¹ by reaction with a compound of general formula (XXXIII):

R¹¹SO₂Cl (XXXIII)

wherein R¹¹ is as defined above. The reaction may be carried out at a temperature of 15 to 30° C., typically room temperature as described in Route 20 of the Examples.

Compounds of general formula (I) in which B is substituted with an ester group —CO₂R¹⁰ may be converted to the equivalent carboxylic acid compounds by hydrolysis, for example alkaline hydrolysis using a base such as lithium hydroxide as shown in Route 24 below.

Compounds of general formula (I) in which B is substituted with an ester group —CO₂R¹⁰ may also be converted to compounds in which the group B is substituted with a CONR¹⁰R¹¹ group, especially a CONH₂ group, by reaction with a hydroxylamine of formula (XXXIV):

NR¹⁰R¹¹OH (XXXIV)

wherein R¹⁰ and R¹¹ are as defined above. The reaction may be conducted in a sealed tube at an elevated temperature, typically about 50° C. Examples of this reaction are given in Routes 26 and 32 below.

Compounds of general formula (I) in which B is substituted with —CO₂H may be converted to compounds of general formula (I) in which B is substituted with an amide group CONR¹⁰R¹¹ by reaction with an amine of formula (XXXV):

R¹⁰OR¹¹NH (XXXV)

wherein R¹⁰ and R¹¹ are as defined above for general formula (I). The reaction is typically carried out in a polar organic solvent such as dichloromethane or N,N-dimethylformamide and at a temperature of about 15 to 30° C., typically room temperature. Examples of this reaction are given in Route 16, where R¹⁰ and R¹¹ together form a cyclic group (in this case morpholine) and in Route 25, where R¹⁰ is hydrogen and R¹¹ is a heteroaromatic group.

Compounds of general formula (I) in which B is substituted with an amide group CONH₂ may also be prepared from compounds of general formula (I) in which B is substituted with a nitrile group by reaction with urea and hydrogen peroxide under mildly basic conditions. The reaction may be conducted in an an aqueous solvent, for example a mixture of acetone and water and at a temperature of 15 to 30° C., typically at room temperature. The reaction is illustrated in the first step of Route 33 below.

Compounds of general formula (I) in which B is substituted by a halogen atom may be converted to compounds of general formula (I) in which B is substituted by a group T by reaction with a boronic acid compound of general formula (XXXVI):

T-B(OH)₂ (XXXVI)

wherein T is as defined for general formula (I) but is preferably an aromatic or heteroaromatic group. The reaction may be carried out using the general method described in Route 29, Example 147 below.

Compounds of general formula (I) in which B is substituted by a halogen atom may be converted to compounds of general formula (I) in which B is substituted by a group NHR¹¹, wherein R¹¹ is as defined above for general formula (I) by reaction with an amine of general formula (XLII) using a suitable palladium phosphine Buchwald-Hartwig catalyst:

NH₂R¹¹ (XLII)

This procedure is illustrated in Route 35 below.

Compounds of general formula (I) in which B is substituted with a group T, where T is a nitrogen-containing heterocyclic group such as piperazine, may be converted to compounds of general formula (I) in which B is substituted with a group T having a —COOR¹³ substituent on a ring nitrogen atom. This conversion may be achieved by reacting the starting material with a chloroformate of general formula (XXXVII):

Cl—C(O)O—R¹³ (XXXVII)

wherein R¹³ is as defined for general formula (I). The reaction may be carried out in an organic solvent such as toluene and at a temperature of about 15 to 40° C., typically room temperature. Examples of this type of reaction are illustrated in Routes 5 and 8 below.

Compounds of general formula (I) in which B is substituted with a group T, where T is a nitrogen-containing heterocyclic group such as piperazine, may also be converted to compounds of general formula (I) in which B is substituted with a a group T having a —CONHR¹³ substituent on a ring nitrogen atom. This can be achieved by reacting the starting material with an isocyanate of formula (XXXVIII):

O=C═N—R¹³ (XXXVIII)

wherein R¹³ is as defined for general formula (I). The reaction may be carried out in an organic solvent such as dichloromethane and at a temperature of about 15 to 40° C., typically room temperature. An example of this type of reaction is illustrated in Route 6 below.

Compounds of general formula (I) in which B is substituted with a group T, where T is a nitrogen-containing heterocyclic group such as piperazine, may also be converted to compounds of general formula (I) in which B is substituted with a a group T having a —COR¹³ substituent on a ring nitrogen atom. This can be achieved by reacting the starting material with an acid chloride of formula (IXL):

Cl—C(O)—R¹³ (IXL)

wherein R¹³ is as defined for general formula (I). The reaction may be carried out in an organic solvent such as dichloromethane with initial cooling, for example to about 0° C. and subsequent warming to a temperature of about 15 to 40° C., typically room temperature. An example of this type of reaction is illustrated in Route 7 below.

Compounds of general formula (I) in which B is substituted with a group T, where T is a nitrogen-containing heterocyclic group such as piperazine, may also be converted to compounds of general formula (I) in which B is substituted with a group T having a methyl substituent on a ring nitrogen atom. This can be achieved by reacting a solution of the starting material in formic acid with formaldehyde as illustrated in Route 9 below.

Compounds of general formula (I) in which the ring B has a heteroaromatic ring substituent can be prepared in a number of ways. For example a compound in which the ring B has an oxadiazole substituent may be prepared from a compound of general formula (I) in which B is substituted with CO₂H by reaction firstly with thionyl chloride to give an acid chloride intermediate which is then reacted with a compound of general formula (XL):

R¹³—C(NH₂)═NOH (XL)

where R¹³ is as defined in general formula (I), but is especially an alkyl group.

The reaction may be conducted at elevated temperature of, for example, about 80 to 140° C., typically 110° C.

The compound of general formula (XL) may be prepared by reacting hydroxylamine with a compound of general formula (XLI):

R¹³—CN (XLI)

where R¹³ is as defined in general formula (I), but is especially an alkyl group. This reaction is illustrated in Routes 27 and 33 below.

Compounds of general formula (I) in which the ring B is substituted with a triazole may be prepared from compounds in which the ring B is substituted with a primary amide group —CONH₂ by reaction with N,N-dimethyl formamide dimethyl acetal followed by hydrazine monohydrate. The reaction is described in Route 33 and Example 159.

Compounds of general formula (I) in which A is substituted by an amide —C(O)NH₂ may be prepared from compounds of similar to those of general formula (I) but in which A is substituted by C(O)OR³, where R³ is as defined above but is especially methyl or ethyl by reaction with aqueous ammonia. The reaction may be conducted in a sealed tube, typically heated to about 50° C.

Compounds of general formula (I) in which A is substituted with NHR³ or NR³R⁴ may be prepared from the equivalent compound of general formula (I) in which A is substituted with NH₂ by reaction with a compound of general formula (XXI):

R³—C(O)H (XXI)

under reducing conditions using an agent such as STAB. If required, the product of this reaction, in which A is substituted with NHR³, can then be reacted with a compound of general formula (XXIII):

R⁴—C(O)H (XXIII)

under reducing conditions using an agent such as STAB to give a compound of general formula (I) in which A is substituted with NR³R⁴.

Compounds in which B has a variety of different substituents may be prepared from the appropriate compounds of general formula (XII) as defined above.

As discussed above, the compounds of formula (XII) are often prepared by reducing the corresponding nitro substituted compound of general formula (XV). However, there are alternative routes for the preparation of compounds of general formula (XII), some of which are methods known to those skilled in the art or adaptations of such methods.

For example, compounds of general formula (XII) in which B is substituted with NR¹⁰C(O)R¹¹ may be prepared from a compound of general formula (XII) substituted with NH—C(O)O^{t}Butyl (NH-Boc) by reaction with a compound of general formula (XXII):

R¹⁰—C(O)H (XXII)

under reducing conditions using an agent such as STAB, followed by reaction with a compound of general formula (XXV):

Cl—C(O)—R¹¹ (XXV)

in the presence of a weak base such as pyridine.

The butoxy carbonyl protecting group can then be removed by acidic cleavage, for example using hydrogen chloride followed by de-salting with a base such as carbonate resin, to give the required compound of general formula (XII).

Compounds of formula (XII) in which the ring B is substituted by NH-Boc can also be converted to compounds of general formula (XII) in which the ring B is substituted with NR¹⁰(CH₂)_{q}C(O)OR¹¹. In this case, the first step is again the reaction of the starting material with a compound of general formula (XXII) under reducing conditions as set out above. However, the product is then reacted with a compound of general formula (XXVI):

X—(CH₂)_{q}C(O)OR¹¹ (XXVI)

where q and R¹¹ are as defined above for general formula (I) and X is a leaving group, especially a halogen such as chlorine.

As before, acidic cleavage of the Boc-protected amine followed by de-salting gives the required compound of general formula (XII).

Compounds of general formula (XII) in which B is substituted by SO₂NR¹⁰R¹¹ may be prepared from a compound of general formula (XXVII):

O₂N—B—SO₂Cl (XXVII)

wherein B is as defined for general formula (I) by reaction with an amine of general formula (XX):

R¹⁰OR¹¹NH (XX)

wherein R¹⁰ and R¹¹ are as defined above in the presence of a mild base such as pyridine.

The intermediate nitro compound can be reduced to give the product compound of general formula (XII), for example by hydrogenation using an appropriate catalyst, such as Raney nickel.

Compounds of general formula (XII) in which the ring B is substituted by C(O)NR¹⁰R¹¹may be prepared from a compound of general formula (XXVIII):

Boc-NH—B—CO₂CH₃ (XXVIII)

wherein B is as defined above for general formula (I) by reaction with a weak base such as lithium hydroxide, followed by reaction with a compound of general formula (XX) as defined above. The Boc protecting group can be removed by acidic cleavage as outlined above to leave the required product of general formula (XII).

Compounds of general formula (XII) in which the ring B is substituted with a CO₂Me group may be prepared from the equivalent carboxylic acids by reaction with TMSCHN₂, for example as described in Route 14 below.

Alternatively, an equivalent reaction can be carried out to convert a compound of general formula (XV) in which the ring B is substituted with a carboxylic acid group to a compound of general formula (XV) with a CO₂R¹⁰ group on the B ring and then reducing the nitro group of the compound of formula (XV) as described above to obtain the desired compound of general formula (XII).

Compounds of general formula (XII) in which B is a thiazole group substituted with a group T may be prepared by reacting a compound of formula (XLII):

T-C(O)—CH₂Br (XLII)

where T is as defined above for general formula (I), with thiourea. Typically the reaction is conducted in a solvent such as ethanol at a raised temperature, for example about 80 to 100° C. This reaction is illustrated in Route 12 below.

A method for the preparation of the novel compounds of general formula (I) forms a further aspect of the present invention.

The compounds of general formula (I) are useful in a method for the treatment of diseases or conditions mediated by the inhibition of GST2, the method comprising administering to a patient in need of such treatment an appropriate amount of a compound of general formula (I).

Therefore, in a further aspect of the invention, there is provided a compound of general formula (I) for use in medicine, especially for the treatment or prevention of metabolic disorders, inflammatory conditions, allergic conditions, fever, pain including allodynia and nociception, eating disorders, cachexia, brain injuries, cancer of the genitals, sleep apnoea, cardiovascular disease, flush effect associated with nicotinic acid and related compounds or for the promotion of wound healing.

Furthermore, the invention also provides the use of a compound of general formula (I) in the preparation of an agent for the treatment or prevention of metabolic disorders, inflammatory conditions, allergic conditions, fever, pain including allodynia and nociception, eating disorders, cachexia, brain injuries, cancer of the genitals, sleep apnoea, cardiovascular disease, flush effect associated with nicotinic acid and related compounds or for the promotion of wound healing.

The metabolic disorder may be metabolic syndrome, a disorder of the lipid or carbohydrate metabolic system, for example, obesity, inflammation associated with obesity, impaired glucose tolerance, diabetes mellitus (particularly diabetes mellitus type I, diabetes mellitus type II and latent autoimmune diabetes in adults) and complications thereof, and lipid disorders and complications thereof.

Examples of complications associated with diabetes include diabetic gangrene, diabetic arthropathy, diabetic osteopenia, diabetic glomerosclerosis, diabetic nephropathy, diabetic dermopathy, diabetic neuropathy, diabetic cataract and diabetic retinopathy, diabetic maculopathy, diabetic feet syndrome, diabetic coma with or without ketoacidosis, diabetic hyperosmolar coma, hypoglycemic coma, hyperglycemic coma, diabetic acidosis, diabetic ketoacidosis, intracapillary glomerulonephrosis, Kimmelstiel-Wilson syndrome, diabetic amyotrophy, diabetic autonomic neuropathy, diabetic mononeuropathy, diabetic polyneuropathy, diabetic angiopathies, diabetic peripheral angiopathy, diabetic ulcer, diabetic arthropathy and obesity in diabetes.

Complications associated with lipid disorders include hypercholesterolemia, familial hypercholesterolemia, Fredrickson's hyperlipoproteinemia, hyperbetalipoproteinemia, hyperlipidemia, low-density-lipoprotein-type [LDL] hyperlipoproteinemia, pure hyperglyceridemia, endogenous hyperglyceridemia, isolated hypercholesterolemia, isolated hypertroglyceridemia, cardiovascular diseases such as hypertension, ischemia, varicose veins, retinal vein occlusion, atherosclerosis, stroke, thrombosis, angina pectoris, myocardial infarction, stenocardia, pulmonary hypertension, congestive heart failure, glomerulopaty, tubulointestitial disorders, renal failure, angiostenosis, or cerebrovascular disorders, such as cerebral apoplexy.

Typical inflammatory conditions which may be treated by the compounds of the present invention are those conditions which are associated with altered prostaglandin profiles. Examples of such conditions include granuloma, systemic lupus erythematosus, inflammatory bowel disease, Crohn's disease, ulcerative colitis, multiple sclerosis and other demylelinating diseases, osteoarthritis, rheumatoid arthritis, psoriatic arthritis, systemic vasculitis, amyotrophic lateral sclerosis (ALS or Lou Gehrig's disease), hypothyroidism, chronic obstructive pulmonary disease (COPD), asthma and psoriasis. The compounds are also useful in the promotion of wound healing and for treating brain injuries.

Allergic conditions which may be treated by the compounds of the present invention include anaphylaxis, allergic rhinitis (hay fever) and mastocytosis.

Although the compounds of general formula (I) may be used to treat any of the conditions specified above, they are particularly suitable for the treatment of obesity; diabetes; metabolic syndrome; lipid disorder; asthma; and allergic rhinitis.

The compounds of general formula (I) may be formulated in an appropriate manner for pharmaceutical use.

Therefore, in a further aspect of the invention there is provided a pharmaceutical composition comprising a compound of general formula (I) together with a pharmaceutical excipient or carrier. The pharmaceutical composition may be in any form suitable for the intended method of administration.

The carrier, or, if more than one be present, each of the carriers, must be acceptable in the sense of being compatible with the other ingredients of the formulation and not deleterious to the recipient.

The compounds of the present invention may be administered orally, parenteraly, such as subcutaneously, intravenously, intramuscularly, intraperitoneally, intrathecally, transdermally, transmucosally, subdurally, locally or topically via iontopheresis, sublingually, by inhalation spray, aerosol or rectally and the like in dosage unit formulations optionally comprising conventional pharmaceutically acceptable excipients.

The compositions may be prepared by bringing into association the above defined active agent with the carrier. In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a novel compound of general formula (I) in conjunction or association with a pharmaceutically or veterinarily acceptable carrier or vehicle.

Particular methods for the production of pharmaceutical compositions are well known to those skilled in the art and are, for example, in Remington's Pharmaceutical Sciences, 15th Ed., Mack Publishing Co., New Jersey (1991).

Formulations for oral administration in the present invention may be presented as: discrete units such as capsules, sachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone

(Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

For topical application to the skin, compounds of general formula (I) or (II) may be made up into a cream, ointment, jelly, solution or suspension etc. Cream or ointment formulations that may be used for the drug are conventional formulations well known in the art, for example, as described in standard text books of pharmaceutics such as the British Pharmacopoeia.

Compounds of general formula (I) may be administered by nasal, bronchial or buccal routes in, for example, the form of aerosols or sprays which can disperse the pharmacological active ingredient in the form of a powder or in the form of drops of a solution or suspension. Pharmaceutical compositions with powder-dispersing properties usually contain, in addition to the active ingredient, a liquid propellant with a boiling point below room temperature and, if desired, adjuncts, such as liquid or solid non-ionic or anionic surfactants and/or diluents. Pharmaceutical compositions in which the pharmacological active ingredient is in solution contain, in addition to this, a suitable propellant, and furthermore, if necessary, an additional solvent and/or a stabiliser. Instead of the propellant, compressed air can also be used, it being possible for this to be produced as required by means of a suitable compression and expansion device.

Parenteral formulations will generally be sterile.

Typically, the preferred dosage of the compound will generally be from about 0.01 to 500 mg/day, preferably from about 0.01 to about 200 mg/kg, and most preferably from about 0.01 to 100 mg/kg; so as to maintain the concentration of drug in the plasma at a concentration effective to treat diabetes. The precise amount of a compound of general formula (I) which is therapeutically effective, and the route by which such compound is best administered, is readily determined by one of ordinary skill in the art by comparing the blood level of the agent to the concentration required to have a therapeutic effect.

The pharmaceutical composition according to the present invention, further may comprise an additional therapeutic agent.

Particularly preferred are compositions, wherein the additional therapeutic agent is selected from antidiabetics like insulin, long and short acting insulin analogues, sulfonylureas and other antidiabetics derived from thiazolidindiones, lipid lowering agents such as statines, fibrates, ion exchange resins, nicotinic acid derivatives, or HMG-CoA reductase inhibitors, cardiovascular therapeutics such as nitrates, antihypertensiva such as β-blockers, ACE inhibitors, Ca-channel blockers, angiotensin II receptor antagonists, diuretics, thrombocyte aggregation inhibitors, or antineoplastic agents such as alkaloids, alkylating agents, antibiotics, or antimetabolites.

More particularly preferred are compounds such as human NPH insulin, human lente or ultralente insulin, insulin Lispro, insulin Asptart, or insulin Glargine, atenolol, bisoprolol, metoprolol, esmolol, celiprolol, talinolol, oxprenolol, pindolol, propanolol, bupropanolol, penbutolol, mepindolol, sotalol, certeolol, nadolol, carvedilol, nifedipin, nitrendipin, amlodipin, nicardipin, nisoldipin, diltiazem, enalapril, verapamil, gallopamil, quinapril, captopril, lisinopril, benazepril, ramipril, peridopril, fosinopril, trandolapril, irbesatan, losartan, valsartan, telmisartan, eprosartan, olmesartan, hydrochlorothiazide, piretanid, chlorotalidone, mefruside, furosemide, bendroflumethiazid, triamterene, dehydralazine, acetylsalicylic acid, tirofiban-HCl, dipyramidol, triclopidin, iloprost-trometanol, eptifibatide, clopidogrel, piratecam, abciximab, trapidil, simvastatine, bezafibrate, fenofibrate, gemfibrozil, etofyllin, clofibrate, etofibrate, fluvastatine, lovastatine, pravastatin, colestyramide, colestipol-HCl, xantinol nicotinat, inositol nicotinat, acipimox, nebivolol, glycerolnitrate, isosorbide mononitrate, isosorbide dinitrate, pentaerythrityl tetranitrate, indapamide, cilazepril, urapidil, eprosartan, nilvadipin, metoprolol, doxazosin, molsidormin, moxaverin, acebutolol, prazosine, trapidil, clonidine, vinca alkaloids and analogues such as vinblastin, vincristin, vindesin, vinorelbin, podophyllotoxine derivatives, etoposid, teniposid, alkylating agents, nitroso ureas, N-lost analogues, cycloplonphamid, estamustin, melphalan, ifosfamid, mitoxantron, idarubicin, doxorubicin, bleomycin, mitomycin, dactinomycin, daptomycin, antimetabolites such as cytarabin, fluorouracil, fluoroarabin, gemcitabin, tioguanin, capecitabin, combinations such as adriamydin/daunorubicin, cytosine arabinosid/cytarabine, 4-HC, or other phosphamides.

The additional therapeutic agent is not necessarily included in the pharmaceutical composition containing the compound of general formula (I).

Therefore, in a further aspect of the invention, there is provided a product comprising a compound of general formula (I) and one or more of the agents listed above as a combined preparation for simultaneous, separate or sequential use in the treatment of a disease or condition as specified above.

There is also provided the use of a compound of general formula (I) as set out above in the preparation of a medicament for the treatment or prevention of metabolic disorders, inflammatory conditions, allergic conditions, fever, pain including allodynia and nociception, eating disorders, cachexia, brain injuries, cancer of the genitals, sleep apnoea, cardiovascular disease, flush effect associated with nicotinic acid and related compounds or for the promotion of wound healing, wherein the medicament further comprises or is co-administered with one of the agents listed above.

The invention will now be further described with reference to the following examples and the drawing in which:

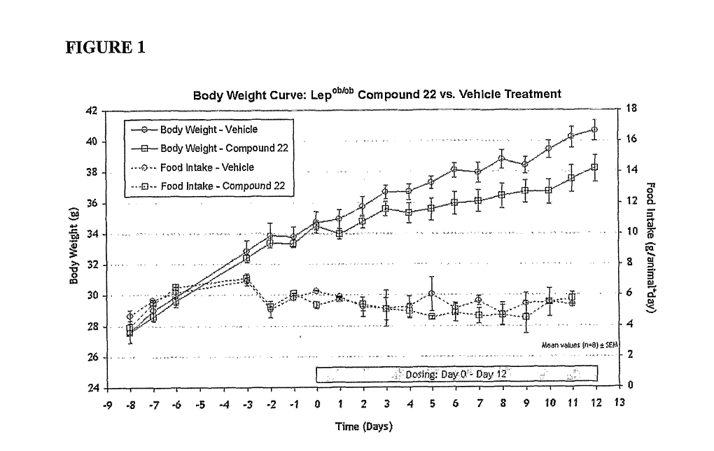

FIG. 1 is a body weight curve graph which plots the body weight and food intake of mice over time. Some of the mice are given food only whereas some are given food together with Compound 22 below.

### EXAMPLES

In the Examples, the following abbreviations are used:

| | |
|---|---|
| DCE dichloroethane | PS-TsOH polymer supported |
| | toluenesulphonic acid |
| DCM dichloromethane | TFA trifluoroacetic acid |
| EtOAc ethyl acetate | IPA isopropyl alcohol |
| STAB sodium trisacetoxyborohydride | NBS N-bromo succinimide |
| EDC (1-(3-dimethylaminopropyl)-3- | DIPEA N,N-Diisopropylethylamine |
| ethyl dicarbodiimide hydrochloride | |
| PS-NCO polymer supported isocyanate | MeOH methanol |
| MeCN acetonitrile | Pd₂(dba)₃ |
| | Tris(dibenzylideneacetone)dipalladium |
| | (0) |
| EtOH ethanol | DMF N,N-dimethylformamide |
| RT room temperature | CDI di-imidazpl-1-yl methanone |
| | (carbonyl di-imidazole) |
| HPLC high performance liquid | TMSCHN₂ |
| chromatography | |
| THF tetrahydrofuran | HOBt butanol |
| DMAP 4-Dimethylaminopyridine | NEt₃ triethylamine |
| TLC thin layer chromatography | HBTU O-(1H-Benzotriazol-1-yl)- |
| | N,N,N′,N′-tetramethyluronium |
| | hexafluorophosphate |
| TEA triethylamine | DPPA diphenylphosphoryl azide |

Analytical Methods

HPLC-MS Analysis

Example compounds and their intermediates were analysed by HPLC-MS using a combination of the following instrumentation: Waters or Micromass ZMD, ZQ or LCT mass spectrometers with a Waters UV and ELS detector. The HPLC conditions are tabulated below. Micromass MassLynx™ Operating Software with OpenLynx™ Browser was used for data acquisition, processing and reporting.

UV-Directed HPLC Purification.

HPLC purification was performed using Gilson Prep LC modules, running on UniPoint software version 1.71 or 3.0 and HPLC conditions tabulated below:

| | HPLC-MS Analysis | | HPLC Purification | |
|---|---|---|---|---|
| | Standard 3.5 minute method | | ‘10 minute method’ | |
| Column | Atlantis dC18 | | Hyperprep HS C18 | |
| | 2.1 × 50 mm, 5 um | | 100 mm × 21.2 mm i.d. | |
| | | | 12 um particle size | |
| Mobile phase | A = Formic acid (aq) 0.1% | | A = TFA (aq) 0.1% | |
| | B = Formic acid (MeCN) 0.1% | | B = TFA (acetonitrile) 0.1% | |
| Flow rate | 1 | ml/min | 30 | ml/min |
| Injection volume | 3 | ul | 1.6 | ml |
| Detector | 215 | nm (nominal) | 215 and 254 nm | |
| Run Time | 3.5 | mins | 10 | mins |

| | | |
|---|---|---|
| Gradient | Time (mins) | % Organic |
| | 0 | 0 |
| | 2.5 | 100 |
| | 2.7 | 100 |
| | 2.71 | 0 |
| | 3.0 | 0 |

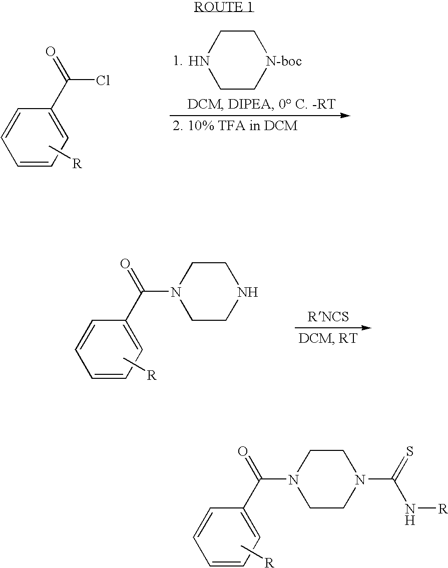

### Example 1

### Preparation of 4-Benzoyl-piperazine-1-carbothioic acid (4-tert-butyl-phenyl)-amide (Compound 1). Potency Range A

### General Procedure A. 4-Benzoyl-piperazine-1-carboxylic acid tert-butyl ester

To a stirred solution of N-boc-piperazine (1 eqv, 0.750 g, 4.03 mmol) and DIPEA (1.2 eqv, 0.84 ml, 4.84 mmol) in DCM (10 ml) at 0° C. was added dropwise benzoyl chloride (1.1 eqv, 0.515 ml, 4.41 mmol). The mixture was warmed to room temperature and stirred for 2 hrs. The reaction mixture was diluted with DCM (15 ml), washed with 1M NaOH aq (25 ml), dried (Na₂SO₄), filtered and evaporated at reduced pressure to give product (1.04 g, 67%) as pale yellow solid. LC/MS: 84% MH⁺, m/z 291, Rt=1.28 mins. The title compound did not require further purification.

### General Procedure B. Phenyl-piperazin-1-yl-methanone

To a solution of (1.0 g, 3.44 mmol) 4-benzoyl-piperazine-1-carboxylic acid tert-butyl ester in dichloromethane (9 ml) was added TFA (1 ml) and the resulting mixture shaken at room temperature for 18 hours. The reaction mixture was concentrated at reduced pressure then partitioned between 1:1 DCM/1M HCl aq (50 ml). The aqueous phase was basified with NaOH solid (until pH 10), extracted with DCM (2×25 ml), dried (Na₂SO₄), filtered and evaporated at reduced pressure. The resulting semi-transparent oil was azeotroped with MeOH (2×10 ml) to give product (348 mg, 53%, 1.83 mmol) as viscous colourless oil. LC/MS: 91% MH⁺, m/z 192, Rt=0.63 mins.

### General Procedure C. 4-Benzoyl-piperazine-1-carbothioic acid (4-tert-butyl-phenyl)-amide

To a solution of (1 eqv, 67 mg, 0.35 mmol) phenyl-piperazin-1-yl-methanone in DCM (1 ml) was added dropwise a solution of (1.0 eqv, 67 mg, 0.35 mmol) 4-tert-butylphenyl isothiocyanate in DCM (1 ml). The resulting mixture was stirred at room temperature for 1 day then concentrated at reduced pressure. The crude material was purified by chromatography [SiO₂, EtOAc/cyclohexane, 1:1] to give pure product (110 mg, 82%, 0.288 mmol) as an off-white solid. LC/MS: 93% MH⁺, m/z 382, Rt=1.50 mins.

The following compounds were made as described in Route 1 Example 1 General procedure C above.

### Example 2

### Preparation of 4-Benzoyl-piperazine-1-carbothioic acid (4-dimethylamino-phenyl)-amide (Compound 2). Potency Range A

In a similar fashion (Route 1:1C, 0.26 mmol of phenyl-piperazin-1-yl-methanone), gave the product (34 mg, 35%, 0.09 mmol) as off-white crystals. LC/MS: 93% MH⁺, m/z 369, Rt=1.49 mins.

### Example 3

### Preparation of 4-Benzoyl-piperazine-1-carbothioic acid (4-nitro-phenyl)-amide (Compound 3). Potency Range B

In a similar fashion (Route 1:1C, 0.26 mmol of phenyl-piperazin-1-yl-methanone), gave the product (78 mg, 81%, 0.21 mmol) as yellow powder. LC/MS: 100% MH⁺, m/z 371, Rt=1.80 mins.

### Example 4

### Preparation of 4-Benzoyl-piperazine-1-carbothioic acid (4-fluoro-phenyl)-amide (Compound 4). Potency Range C

In a similar fashion (Route 1:1C, 0.35 mmol of phenyl-piperazin-1-yl-methanone), gave the product (98 mg, 82%, 0.29 mmol) as white powder. LC/MS: 94% MH⁺, m/z 344, Rt=1.24 mins.

### Example 5

### Preparation of 4-Benzoyl-piperazine-1-carbothioic acid naphthalen-1-ylamide (Compound 5). Potency Range B

In a similar fashion (Route 1:1C, 0.32 mmol of phenyl-piperazin-1-yl-methanone), gave the product (50 mg, 41%, 0.133 mmol) as colourless oil. LC/MS: 98% MH⁺, m/z 376, Rt=1.40 mins.

### Example 6

### Preparation of 4-[(4-Benzoyl-piperazine-1-carbothioyl)-amino]-benzoic acid methyl ester (Compound 6). Potency Range B

In a similar fashion (Route 1:1C, 0.26 mmol of phenyl-piperazin-1-yl-methanone), gave the product (74 mg, 74%, 0.19 mmol) as white solid. LC/MS: 100% MH⁺, m/z 384, Rt=1.74 mins.

### Example 7

### Preparation of 4-Benzoyl-piperazine-1-carbothioic acid p-tolylamide (Compound 7). Potency Range C

In a similar fashion (Route 1:1C, 0.26 mmol of phenyl-piperazin-1-yl-methanone), gave the product (81 mg, 92%, 0.24 mmol) as white crystalline solid. LC/MS: 96% MH⁺, m/z 340, Rt=1.75 mins.

### Example 8

### Preparation of 4-(4-Fluoro-benzoyl)-piperazine-1-carbothioic acid (4-tert-butyl-phenyl)-amide (Compound 8). Potency Range B

### 4-(4-Fluoro-benzoyl)-piperazine-1-carboxylic acid tert-butyl ester

Following general procedure A (to scale 4.41 mmol 4-fluoro-benzoyl chloride) the title compound (0.816 g, 60%, 2.65 mmol) was obtained as an off-white solid. LC/MS: 82% MH⁺, m/z 309, Rt=1.31 mins. The title compound did not require further purification.

### (4-Fluoro-phenyl)-piperazin-1-yl-methanone

Following general procedure B (to scale 2.65 mmol 4-(4-fluoro-benzoyl)-piperazine-1-carboxylic acid tert-butyl ester) the title compound (0.331 g, 60%, 1.59 mmol) was isolated as a white crystalline solid. LC/MS: 91% MH⁺, m/z 209, Rt=0.68 mins. The title compound did not require further purification.

### 4-(4-Fluoro-benzoyl)-piperazine-1-carbothioic acid (4-tert-butyl-phenyl)-amide

In a similar fashion (Route 1:1C, 0.366 mmol of (4-fluoro-phenyl)-piperazin-1-yl-methanone), gave the product (114 mg, 96%, 0.351 mmol) as an off-white solid. LC/MS: 96% MH⁺, m/z 400, Rt=1.50 mins.

The following compounds were made in a similar way as described in Route 1 Example 1 General procedure C above.

### Example 9

### Preparation of 4-(4-Fluoro-benzoyl)-piperazine-1-carbothioic acid (4-phenoxy-phenyl)-amide (Compound 9). Potency Range C

In a similar fashion (Route 1:1C, 0.22 mmol of (4-fluoro-phenyl)-piperazin-1-yl-methanone), gave the product (94 mg, 100%, 0.22 mmol) as white solid. LC/MS: 98% MH⁺, m/z 436, Rt=1.52 mins.

### Example 10

### Preparation of 4-(4-Fluoro-benzoyl)-piperazine-1-carbothioic acid (4-dimethylamino-phenyl)-amide (Compound 10). Potency Range C

In a similar fashion (Route 1:1C, 0.24 mmol of (4-fluoro-phenyl)-piperazin-1-yl-methanone), gave the product (92 mg, 99%, 0.24 mmol) as white crystals. LC/MS: 98% MH⁺, m/z 387, Rt=1.52 mins.

### Example 11

### Preparation of 4-{[4-(4-Fluoro-benzoyl)-piperazine-1-carbothioyl]-amino}-benzoic acid methyl ester (Compound 11). Potency Range C

In a similar fashion (Route 1:1C, 0.24 mmol of (4-fluoro-phenyl)-piperazin-1-yl-methanone), gave the product (96 mg, 100%, 0.24 mmol) as white solid. LC/MS: 100% MH⁺, m/z 402, Rt=1.75 mins.

### Example 12

### Preparation of 4-(4-Fluoro-benzoyl)-piperazine-1-carbothioic acid (4-methoxy-phenyl)-amide (Compound 12). Potency Range C

In a similar fashion (Route 1:1C, 0.366 mmol of (4-fluoro-phenyl)-piperazin-1-yl-methanone), gave the product (121 mg, 95%, 0.347 mmol) as white solid. LC/MS: 95% MH⁺, m/z 374, Rt=1.21 mins.

### Example 13

### Preparation of 4-(4-Fluoro-benzoyl)-piperazine-1-carbothioic acid p-tolylamide (Compound 13). Potency Range C

In a similar fashion (Route 1:1C, 0.366 mmol of (4-fluoro-phenyl)-piperazin-1-yl-methanone), gave the product (108 mg, 94%, 0.344 mmol) as white solid. LC/MS: 94% MH⁺, m/z 358, Rt=1.29 mins.

### Example 14

### Preparation of 4-(Pyridine-3-carbonyl)-piperazine-1-carbothioic acid (4-dimethylamino-phenyl)-amide (Compound 14). Potency Range C

### 4-(Pyridine-3-carbonyl)-piperazine-1-carboxylic acid tert-butyl ester

Following general procedure A (to scale 10.73 mmol nicotinoyl chloride.hydrochloride) the title compound (2.412 g, 77%, 8.28 mmol) was isolated as colourless oil. LC/MS: 100% MH⁺, m/z 292, Rt=0.89 mins. The title compound did not require further purification.

### Piperazin-1-yl-pyridin-3-yl-methanone

Following general procedure B (to scale 8.28 mmol 4-(pyridine-3-carbonyl)-piperazine-1 -carboxylic acid tert-butyl ester) the title compound (1.343 g, 85%, 7.02 mmol) was isolated as pale yellow oil. LC/MS: 96% MH⁺, m/z 192, Rt=0.21 mins. The title compound did not require further purification.

### 4-(Pyridine-3-carbonyl)-piperazine-1-carbothioic acid (4-dimethylamino-phenyl)-amide

In a similar fashion (Route 1:1C, 0.266 mmol of piperazin-1-yl-pyridin-3-yl-methanone), gave the product (83 mg, 85%, 0.225 mmol) as an off-white solid. LC/MS: 100% MH⁺, m/z 370, Rt=0.67 mins.

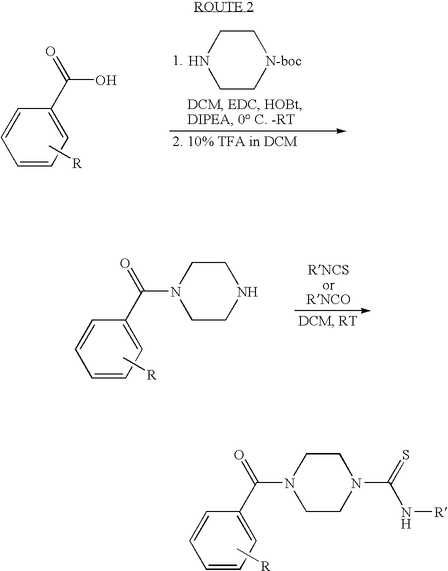

### Example 15

### Preparation of 4-(6-fluoro-pyridine-2-carbonyl)-piperazine-1-carbothioic acid (6-morpholin-4-yl-pyridin-3-yl)-amide (Compound 15). Potency Range C

### General Procedure D. 4-(6-Fluoro-pyridine-2-carbonyl)-piperazine-1-carboxylic acid tert-butyl ester

To a stirred solution of 6-fluoro-2-pyridine carboxylic acid (1 eqv, 2.00 g, 14.2 mmol), N-boc-piperazine (1.15 eqv, 3.04 g, 16.3 mmol) and DIPEA (2.5 eqv, 6.12 ml, 35.4 mmol) in DCM (100 ml) at room temperature was added portionwise EDC (1.2 eqv, 3.09 g, 17.0 mmol) and HOBt (0.2 eqv, 0.383 g, 2.83 mmol). The reaction mixture was stirred at room temperature for 16 hrs then concentrated at reduced pressure. The crude material was purified by chromatography [SiO₂, EtOAc/heptane, 1:1] to give pure product (4.40 g, 84%, 11.93 mmol) as colourless oil. LC/MS: 95% MH⁺, m/z 310, Rt=0.97 mins.

### General Procedure E. 6-Fluoro-pyridin-2-yl)-piperazin-1-yl-methanone

To a solution of (1 eqv, 4.4 g, 14.2 mmol) 4-(6-fluoro-pyridine-2-carbonyl)-piperazine-1-carboxylic acid tert-butyl ester in dioxane (10 ml) was added 4M HCl in dioxane (10 eqv, 35.6 ml, 35.6 mmol) and the resulting mixture stirred at room temperature for 18 hours. The reaction mixture was concentrated at reduced pressure and the residue taken up in DCM (100 ml), carbonate resin (4 eqv, 19 g, 56.9 mmol) added and the reaction mixture shaken at room temperature for 16 hrs. The resin was removed by filtration and the solvent evaporated under reduced pressure to give product (2.70 g, 91%, 10.85 mmol) as a white solid. LC/MS: >85% MH⁺, m/z 210, Rt=0.58 mins. The title compound did not require further purification.

### General Procedure F. 4-(6-Fluoro-pyridine-2-carbonyl)-piperazine-1-carbothioic acid (6-morpholin-4-yl-pyridin-3-yl)-amide (Compound 16)

To a solution of (1.2 eqv, 63.46 mg, 0.29 mmol) title compound E, 6-fluoro-pyridin-2-yl)-piperazin-1-yl-methanone, in DCM (1 ml) was added dropwise a solution of (1.0 eqv, 50 mg, 0.239 mmol) 4-(5-isothiocyanato-pyridin-2-yl)-morpholine in DCM (1 ml). The resulting mixture was stirred at room temperature for 16 hrs then concentrated at reduced pressure. The crude material was purified by chromatography [SiO₂, EtOAc/heptane, 1:1] to give pure product (125 mg, 73%, 0.032 mmol) as a white solid. LC/MS: 100% MH⁺, m/z 423, Rt=1.22 mins.

The following compounds were made as described in Route 2 Example 15 General procedure F above.

### Example 17

### Preparation of 4-(6-Fluoro-pyridine-2-carbonyl)-piperazine-1-carbothioic acid (4-phenoxy-phenyl)-amide (Compound 17). Potency Range C

In a similar fashion (Route 2:15F, 0.22 mmol of (6-fluoro-pyridin-2-yl)-piperazin-1-yl-methanone), gave the product (29 mg, 30%, 0.066 mmol) as white solid. LC/MS: 100% MH⁺, m/z 437, Rt=1.42 mins.

### Example 18

### Preparation of 4-(6-Fluoro-pyridine-2-carbonyl)-piperazine-1-carbothioic acid (4-difluoromethoxy-phenyl)-amide (Compound 18). Potency Range C

In a similar fashion (Route 2:15F, 0.239 mmol of (6-fluoro-pyridin-2-yl)-piperazin-1-yl-methanone), gave the product (42 mg, 43%, 0.103 mmol) as white solid. LC/MS: 100% MH⁺, m/z 411, Rt=1.28 mins.

### Example 19

### Preparation of 4-(6-Fluoro-pyridine-2-carbonyl)-piperazine-1-carbothioic acid (4-trifluoromethoxy-phenyl)-amide (Compound 19). Potency Range C

In a similar fashion (Route 2:15F, 0.239 mmol of (6-fluoro-pyridin-2-yl)-piperazin-1-yl-methanone), gave the product (40 mg, 39%, 0.093 mmol) as white solid. LC/MS: 100% MH⁺, m/z 429, Rt=1.40 mins.

### Example 20

### Preparation of 4-(6-Fluoro-pyridine-2-carbonyl)-piperazine-1-carbothioic acid (4-trifluoromethyl-phenyl)-amide (Compound 20). Potency Range C

In a similar fashion (Route 2:15F, 0.239 mmol of (6-fluoro-pyridin-2-yl)-piperazin-1-yl-methanone), gave the product (42 mg, 42%, 0.10 mmol) as white solid. LC/MS: 100% MH⁺, m/z 413, Rt=1.38 mins.

The following compound was made as described in Route 2 Example 15 General procedure F, using 1-isocyanato-4-trifluoromethoxy-benzene.

### Example 21

### Preparation of 4-(6-Fluoro-pyridine-2-carbonyl)-piperazine-1-carboxylic acid (4-trifluoromethoxy-phenyl)-amide (Compound 21). Potency Range C

In a similar fashion (Route 2:15F, 0.239 mmol of (6-fluoro-pyridin-2-yl)-piperazin-1-yl-methanone), gave the product (60 mg, 61%, 0.146 mmol) as white solid. LC/MS: 100% MH⁺, m/z 413, Rt=1.32 mins.

The following compound was made as described in Route 2 Example 15 General procedure F, using 1-isocyanato-4-trifluoromethyl-benzene.

### Example 22

### Preparation of 4-(6-Fluoro-pyridine-2-carbonyl)-piperazine-1-carboxylic acid (4-trifluoromethyl-phenyl)-amide (Compound 22). Potency Range C

In a similar fashion (Route 2:15F, 0.239 mmol of (6-fluoro-pyridin-2-yl)-piperazin-1-yl-methanone), gave the product (51 mg, 54%, 0.129 mmol) as white solid. LC/MS: 100% MH⁺, m/z 397, Rt=1.38 mins.

The following compounds were made as described in Route 1 Example 1 General procedures A, B and C, using 3-fluoro-benzoyl chloride instead of benzoyl chloride.

### Example 23

### Preparation of 4-(3-Fluoro-benzoyl)-piperazine-1-carbothioic acid (4-trifluoromethyl-phenyl)-amide (Compound 23). Potency Range B

### 4-(3-Fluoro-benzoyl)-piperazine-1-carboxylic acid tert-butyl ester

Following general procedure A (to scale 16.1 mmol 3-fluoro-benzoyl chloride) the title compound (4.699 g, 95%, 15.23 mmol) was obtained as an off-white solid. LC/MS: 97% MH⁺, m/z 309, Rt=1.37 mins. The title compound did not require further purification.

### (3-Fluoro-phenyl)-piperazin-1-yl-methanone

Following general procedure B (to scale 15.23 mmol 4-(3-fluoro-benzoyl)-piperazine-1-carboxylic acid tert-butyl ester) the title compound (3.14 g, 99%, 15.08 mmol) was isolated as a white crystalline solid. LC/MS: not ionised. The title compound was pure by ¹H NMR so did not require further purification.

### 4-(3-Fluoro-benzoyl)-piperazine-1-carbothioic acid (4-trifluoromethyl-phenyl)-amide

In a similar fashion (Route 1:1C, 1.00 mmol of (3-fluoro-phenyl)-piperazin-1-yl-methanone), gave the product (209 mg, 51%, 0.51 mmol) as an off-white solid. LC/MS: 97% MH⁺, m/z 412, Rt=2.27 mins.

The following compounds were made in a similar way as described in Route 1 Example 1 General procedure C above.

### Example 24

### Preparation of 4-(3-Fluoro-benzoyl)-piperazine-1-carboxylic acid (2,6-dichloro-pyridin-4-yl)-amide (Compound 24). Potency Range C

In a similar fashion (Route 1:1C, 0.24 mmol of (3-fluoro-phenyl)-piperazin-1-yl-methanone), gave the product (60 mg, 63%, 0.15 mmol) as an off-white solid. LC/MS: 100% MH⁺, m/z 397, Rt=1.92 mins.

### Example 25

### Preparation of 4-(3-Fluoro-benzoyl)-piperazine-1-carboxylic acid (3-phenoxy-phenyl)-amide (Compound 25). Potency Range B

In a similar fashion (Route 1:1C, 0.24 mmol of (3-fluoro-phenyl)-piperazin-1-yl-methanone), gave the product (80 mg, 80%, 0.19 mmol) as an off-white solid. LC/MS: 100% MH⁺, m/z 420, Rt=2.06 mins.

### Example 26

### Preparation of 4-(3-Fluoro-benzoyl)-piperazine-1-carboxylic acid (4-phenoxy-phenyl)-amide (Compound 26). Potency Range B

In a similar fashion (Route 1:1C, 0.17 mmol of (3-fluoro-phenyl)-piperazin-1-yl-methanone), gave the product (69 mg, 97%, 0.17 mmol) as an off-white solid. LC/MS: 97% MH⁺, m/z 420, Rt=2.21 mins.

### Example 27

### Preparation of 4-(3-Fluoro-benzoyl)-piperazine-1-carboxylic acid benzo[b]thiophen-5-ylamide (Compound 27). Potency Range B

In a similar fashion (Route 1:1C, 0.17 mmol of (3-fluoro-phenyl)-piperazin-1-yl-methanone), gave the product (29 mg, 45%, 0.07 mmol) as an off-white solid. LC/MS: 97% MH⁺, m/z 384, Rt=2.03 mins.

### Example 28

### Preparation of 4-(3-Fluoro-benzoyl)-piperazine-1-carboxylic acid (4-thiophen-2-yl-phenyl)-amide (Compound 28). Potency Range B

In a similar fashion (Route 1:1C, 0.17 mmol of (3-fluoro-phenyl)-piperazin-1-yl-methanone), gave the product (45 mg, 66%, 0.11 mmol) as an off-white solid. LC/MS: 100% MH⁺, m/z 410, Rt=2.16 mins.

### Example 29

### Preparation of 4-(3-Fluoro-benzoyl)-piperazine-1-carboxylic acid [4-(2-methyl-thiazol-3-yl)-phenyl]-amide (Compound 29). Potency Range B

In a similar fashion (Route 1:1C, 0.17 mmol of (3-fluoro-phenyl)-piperazin-1-yl-methanone), gave the product (67 mg, 93%, 0.16 mmol) as an off-white solid. LC/MS: 100% MH⁺, m/z 425, Rt=1.99 mins.

### Example 30

### Preparation of 4-(3-Fluoro-benzoyl)-piperazine-1-carboxylic acid [3-(5-methyl-[1,2,4]oxadiazol-3-yl)-phenyl]-amide (Compound 30). Potency Range B

In a similar fashion (Route 1:1C, 0.17 mmol of (3-fluoro-phenyl)-piperazin-1-yl-methanone), gave the product (47 mg, 67%, 0.11 mmol) as an off-white solid. LC/MS: 95% MH⁺, m/z 410, Rt=1.93 mins.

### Example 31

### Preparation of 4-(3-Fluoro-benzoyl)-piperazine-1-carboxylic acid (3-morpholin-4-yl-phenyl)-amide (Compound 31). Potency Range C

In a similar fashion (Route 1:1C, 0.23 mmol of (3-fluoro-phenyl)-piperazin-1-yl-methanone), gave the product (3 mg, 3%, 0.01 mmol) as an off-white solid. LC/MS: 96% MH⁺, m/z 413, Rt=1.75 mins.

### Example 32

### Preparation of 4-(3-Fluoro-benzoyl)-piperazine-1-carboxylic acid (3-bromo-phenyl)-amide (Compound 32). Potency Range B

In a similar fashion (Route 1:1C, 2.59 mmol of (3-fluoro-phenyl)-piperazin-1-yl-methanone), gave the product (1.0 g, 95%, 2.46 mmol) as a white solid. LC/MS: 100% MH⁺, m/z 407/409, Rt=2.10 mins.

### Example 33

### Preparation of 4-{[4-(3-Fluoro-benzoyl)-piperazine-1-carbonyl]-amino}-benzoic acid ethyl ester (Compound 33). Potency Range C

In a similar fashion (Route 1:1C, 1.20 mmol of (3-fluoro-phenyl)-piperazin-1-yl-methanone), gave the product (0.425 g, 89%, 1.07 mmol) as a white solid. LC/MS: 100% MH⁺, m/z 400, Rt=1.90 mins.

### Example 34

### Preparation of 3-{[4-(3-Fluoro-benzoyl)-piperazine-1-carbonyl]-amino}-benzoic acid methyl ester (Compound 34). Potency Range B

In a similar fashion (Route 1:1C, 5.35 mmol of (3-fluoro-phenyl)-piperazin-1-yl-methanone), gave the product (1.40 g, 68%, 3.64 mmol) as a white solid. LC/MS: 100% MH⁺, m/z 386, Rt=1.95 mins.

### Example 35

### Preparation of 4-(3-Fluoro-benzoyl)-piperazine-1-carboxylic acid (3-cyano-phenyl)-amide (Compound 35). Potency Range B

In a similar fashion (Route 1:1C, 8.92 mmol of (3-fluoro-phenyl)-piperazin-1-yl-methanone), gave the product (3.206 g, 97%, 9.1 mmol) as a white solid. LC/MS: 97% MH⁺, m/z 353, Rt=1.92 mins.

### Example 36

### Preparation of 4-(3-Fluoro-benzoyl)-piperazine-1-carboxylic acid (3-nitro-phenyl)-amide (Compound 36). Potency Range C

In a similar fashion (Route 1:1C, 2.4 mmol of (3-fluoro-phenyl)-piperazin-1-yl-methanone), gave the product (0.653 g, 73%, 1.76 mmol) as a yellow solid. LC/MS: 100% MH⁺, m/z 373, Rt=2.02 mins.

### Example 37

### Preparation of 4-(3-Fluoro-benzoyl)-piperazine-1-carboxylic acid (4-nitro-phenyl)-amide (Compound 37). Potency Range C

In a similar fashion (Route 1:1C, 1.44 mmol of (3 -fluoro-phenyl)-piperazin-1-yl-methanone), gave the product (0.430 g, 80%, 1.16 mmol) as a yellow solid. LC/MS: 100% MH⁺, m/z 373, Rt=1.87 mins.

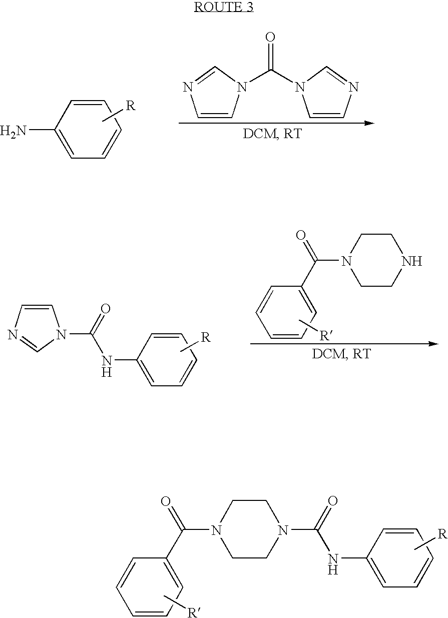

### Example 38

### Preparation of 4-(3-Fluoro-benzoyl)-piperazine-1-carboxylic acid (3-chloro-4-morpholin-4-yl-phenyl)-amide (Compound 38). Potency Range B

### General Procedure G. 4-Benzyl-piperazine-1-carbothioic acid (4-hydroxy-phenyl)-amide

To a stirred solution of 3-chloro-4-morpholin-4-yl-phenylamine (1 eqv, 0.051 g, 0.24 mmol) in DCM (2 ml) at room temperature was added di-imidazol-1-yl-methanone (1 eqv, 0.039 g, 0.24 mmol). The reaction mixture was stirred for 12 hrs. The formation of intermediate, imidazole-1-carboxylic acid (3-chloro-4-morpholin-4-yl-phenyl)-amide, was monitored by LC/MS: 93% [M+H₂O]⁺, m/z 324, Rt=2.14 mins. To the reaction mixture was added (3-fluoro-phenyl)-piperazin-1-yl-methanone (1 eqv, 0.05 g, 0.24 mmol) and the resulting solution stirred at room temperature for 6 hours then concentrated at reduced pressure. The crude material was purified by chromatography [SiO₂, EtOAc/heptane, 2:1] to give pure product (33 mg, 30%, 0.074 mmol) as colourless oil. LC/MS: 100% MH⁺, m/z 447, Rt=1.84 mins.

The following compounds were made as described in Route 3 Example 38 General procedure G above.

### Example 39

### Preparation of 4-(3-Fluoro-benzoyl)-piperazine-1-carboxylic acid (5-chloro-benzooxazol-2-yl)-amide (Compound 39). Potency Range C

In a similar fashion (Route 3:38G, 0.25 mmol of 5-chloro-benzooxazol-2-ylamine), gave the product (6 mg, 6%, 0.01 mmol) as a white solid after preparative HPLC purification. LC/MS: 90% MH⁺, m/z 403, Rt=1.99 mins.

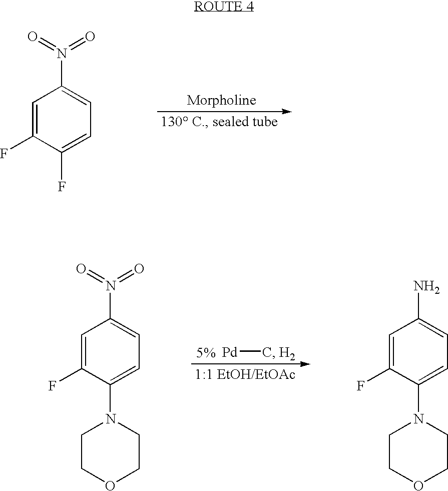

### Preparation of 4-(6-Fluoro-pyridin-2-ylmethyl)-piperazine-1-carbothioic acid (3-fluoro-4-morpholin-4-yl-phenyl)-amide

### General Procedure H. 4-(2-Fluoro-4-nitro-phenyl)-morpholine

A pressure tube was charged with 1,2-difluoro-4-nitro-benzene (1 eqv, 1.5 g, 9.43 mmol) and morpholine (3.5 eqv, 2.87 g, 33.0 mmol) sealed and the reaction mixture stirred for 16 hrs at 130° C. then cooled to room temperature. The resulting precipitate was collected by filtration and washed with heptane (5×5 ml) then purified by chromatography [SiO₂, 25 g isolute cartridge, 95:5 DCM/MeOH] to give product (1.93 g, 91%, 8.54 mmol) as a yellow solid. LC/MS: 100% MH⁺, m/z 227, Rt=1.33 mins.

### General Procedure I. 3-Fluoro-4-morpholin-4-yl-phenylamine

To a solution of 4-(2-fluoro-4-nitro-phenyl)-morpholine (1 eqv, 1.675 g, 8.54 mmol) in EtOH (40 ml) at room temperature under a nitrogen atmosphere was added palladium on carbon (10% by Wt, 0.200 g). The reaction suspension was placed under a hydrogen atmosphere (ca. 1 atm) and stirred at room temperature for 18 hrs. The black suspension was filtered through celite, to remove the palladium on carbon, and evaporated at reduced pressure to give the desired product as a grey powder (1.656 g, 99%, 8.44 mmol). LC/MS: 100% MH⁺, m/z 197, Rt≈0.20 mins.

The following compound was made as described in Route 3 Example 38 General procedure G, using 3-fluoro-4-morpholin-4-yl-phenylamine, di-imidazol-1-yl-methanethione and (6-fluoro-pyridin-2-yl)-piperazin-1-yl-methanone intermediate.

### Example 40

### Preparation of 4-(6-Fluoro-pyridine-2-carbonyl)-piperazine-1-carbothioic acid (3-fluoro-4-morpholin-4-yl-phenyl)-amide (Compound 40). Potency Range C

In a similar fashion (Route 3:38G, 0.22 mmol of 3-fluoro-4-morpholin-4-yl-phenylamine), gave the product (4 mg, 4%, 0.01 mmol) as a colourless oil. LC/MS: 100% MH⁺, m/z 448, Rt=1.73 mins.

The following compounds were made as described in Route 3 Example 38 General procedure G, using the appropriate aniline, di-imidazol-1-yl-methasone and (3-fluoro-phenyl)-piperazin-1-yl-methanone.

### Example 41

### Preparation of 4-(3-Fluoro-benzoyl)-piperazine-1-carboxylic acid (3-fluoro-4-morpholin-4-yl-phenyl)-amide (Compound 41). Potency Range B

In a similar fashion (Route 3:38G, 0.22 mmol of 3-fluoro-4-morpholin-4-yl-phenylamine), gave the product (20 mg, 23%, 0.05 mmol) as a colourless oil. LC/MS: 100% MH⁺, m/z 431, Rt=1.19 mins.

### Example 42

### Preparation of 4-(3-Fluoro-benzoyl)-piperazine-1-carboxylic acid (4-benzoyl-phenyl)-amide (Compound 42). Potency Range B

In a similar fashion (Route 3:38G, 0.15 mmol of (4-amino-phenyl)-phenyl-methanone), gave the product (8 mg, 13%, 0.02 mmol) as a colourless oil. LC/MS: 100% MH⁺, m/z 432, Rt=2.13 mins.

### Example 43

### Preparation of 4-(3-Fluoro-benzoyl)-piperazine-1-carboxylic acid (4-bromo-3-pyridin-4-yl-phenyl)-amide (Compound 43). Potency Range B

In a similar fashion (Route 3:38G, 1.01 mmol of 4-bromo-3-pyridin-4-yl phenylamine), gave the product (112 mg, 23%, 0.23 mmol) as a white solid. LC/MS: 100% MH⁺, m/z 483/485, Rt=1.64 mins.

### Example 44

### Preparation of 4-(3-Fluoro-benzoyl)-piperazine-1-carboxylic acid [6-(pyridin-3-yloxy)-pyridin-3-yl]-amide (Compound 44). Potency Range C

In a similar fashion (Route 3:38G, 1.01 mmol of 6-(pyridin-3-yloxy)-pyridin-3-ylamine), gave the product (124 mg, 29%, 0.29 mmol) as a white solid. LC/MS: 100% MH⁺, m/z 422, Rt=1.58 mins.

### Example 45

### Preparation of 4-(3-Fluoro-benzoyl)-piperazine-1-carboxylic acid quinolin-3-ylamide (Compound 45). Potency Range C

In a similar fashion (Route 3:38G, 1.0 mmol of quinolin-3-ylamine), gave the product (166 mg, 44%, 0.44 mmol) as a white solid. LC/MS: 100% MH⁺, m/z 379, Rt=1.62 mins.

The following compound was made as described in Route 4 Example 40 General procedures H and I, followed by Route 3 Example 38 General procedure G.

### Example 46

### Preparation of 4-(2-Fluoro-4-{[4-(3-fluoro-benzoyl)-piperazine-1-carbonyl]-amino}-phenyl)-piperazine-1-carboxylic acid tert-butyl ester (Compound 46). Potency Range B

### 4-(2-Fluoro-4-nitro-phenyl)-piperazine-1-carboxylic acid tert-butyl ester

Following general procedure H (to scale 1 eqv, 1.5 g, 9.43 mmol 1,2-difluoro-4-nitro-benzene) the title compound (2.90 g, 95%, 8.92 mmol) was obtained as a yellow solid. LC/MS: 100% [M-^{t}Bu]2H⁺, m/z 270, Rt=1.62 mins.

### 4-(4-Amino-2-fluoro-phenyl)-piperazine-1-carboxylic acid tert-butyl ester

Following general procedure I (to scale 1 eqv, 2.90 g, 8.92 mmol 4-(2-fluoro-4-nitro-phenyl)-piperazine-1-carboxylic acid tert-butyl ester) the title compound (6.69 g, 90%, 6.03 mmol) was obtained as a grey solid. LC/MS: 100% [M-^{t}Bu]2H⁺, m/z 238, Rt=0.61 mins.

### 4-(2-Fluoro-4-{[4-(3-fluoro-benzoyl)-piperazine-1-carbonyl]-amino}-phenyl)-piperazine-1-carboxylic acid tert-butyl ester

In a similar fashion (Route 3:38G, 1.35 mmol of 4-(4-amino-2-fluoro-phenyl)-piperazine-1-carboxylic acid tert-butyl ester), gave the product (0.384 g, 54%, 0.73 mmol) as an off-white solid. LC/MS: 100% MH⁺, m/z 530, Rt=2.12 mins.

The following compounds were made as described in Route 1 Example 1 General procedures A, B and C using 2,5-difluoro-benzoyl chloride instead of benzoyl chloride, followed by Route 3 Example 38 General procedure G, using di-imidazol-1-yl-methanone.

### Example 47

### Preparation of 4-(2,5-Difluoro-benzoyl)-piperazine-1-carboxylic acid (3-fluoro-4-morpholin-4-yl-phenyl)-amide (Compound 47). Potency Range C

### 4-(2,5-Difluoro-benzoyl)-piperazine-1-carboxylic acid tert-butyl ester

Following general procedure A (to scale 21.57 mmol 2,5-difluoro-benzoyl chloride) the title compound (6.40 g, 91%, 19.63 mmol) was obtained as a white solid. LC/MS: 100% [M-^{t}Bu]2H⁺, m/z 271, Rt=2.03 mins. The title compound did not require further purification.

### (2,5-Difluoro-phenyl)-piperazin-1-yl-methanone

Following general procedure B (to scale 19.51 mmol 4-(2,5-difluoro-benzoyl)-piperazine-1-carboxylic acid tert-butyl ester) the title compound (3.66 g, 83%, 16.19 mmol) was isolated as a white crystalline solid. LC/MS: 100% MH⁺, m/z 227, Rt=broad 0.75 mins.

### 4-(2,5-Difluoro-benzoyl)-piperazine-1-carboxylic acid (3-fluoro-4-morpholin-4-yl-phenyl)-amide

In a similar fashion (Route 3:38G, 0.3 mmol of 3-fluoro-4-morpholin-4-yl-phenylamine), gave the product (55 mg, 41%, 0.12 mmol) as a white solid. LC/MS: 98% MH⁺, m/z 449, Rt=1.89 mins.

The following compound was made in a similar way as described in Route 3 Example 38 General procedure G above.

### Example 48

### Preparation of 4-(4-{[4-(2,5-Difluoro-benzoyl)-piperazine-1-carbonyl]-amino}-2-fluoro-phenyl)-piperazine-1-carboxylic acid tert-butyl ester (Compound 48). Potency Range C

In a similar fashion (Route 3:38G, 0.30 mmol of 4-(4-amino-2-fluoro-phenyl)-piperazine-1-carboxylic acid tert-butyl ester), gave the product (0.110 g, 68%, 0.2 mmol) as an off-white solid. LC/MS: 100% MH⁺, m/z 548, Rt=2.31 mins.

The following compound was synthesised as described in Route 1 Example 1 General procedure C using (2,5-difluoro-phenyl)-piperazin-1-yl-methanone

### Example 49

### Preparation of 4-(2,5-Difluoro-benzoyl)-piperazine-1-carboxylic acid (4-nitro-phenyl)-amide (Compound 49). Potency Range C

In a similar fashion (Route 1:1C, 1.77 mmol of (2,5-difluoro-phenyl)-piperazin-1-yl-methanone), gave the product (0.690 g, 100%, 1.77 mmol) as a yellow solid. LC/MS: 96% MH⁺, m/z 391, Rt=2.02 mins.

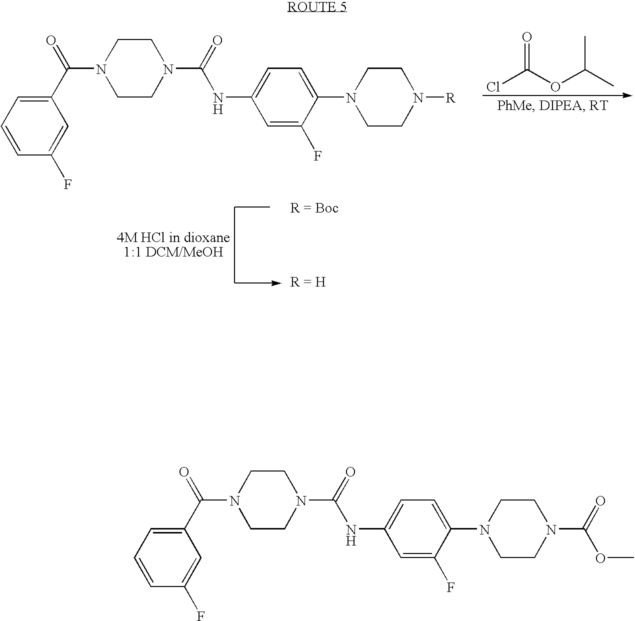

### Example 50

### Preparation of 4-(2-Fluoro-4-{[4-(3-fluoro-benzoyl)-piperazine-1-carbonyl]-amino}-phenyl)-piperazine-1-carboxylic acid isopropyl ester (Compound 50). Potency Range B

### General Procedure J. 4-(3-Fluoro-benzoyl)-piperazine-1-carboxylic acid (3-fluoro-4-piperazin-1-yl-phenyl)-amide

To a stirred 1:1 DCM/MeOH (2 ml) solution of 4-(2-fluoro-4-{[4-(3-fluoro-benzoyl)-piperazine-1-carbonyl]-amino}-phenyl)-piperazine-1-carboxylic acid tert-butyl ester (1 eqv, 0.135 g, 0.255 mmol) was added 4M HCl in dioxane (4 eqv, 0.25 ml, 1.0 mmol) and the resulting mixture stirred at room temperature for 12 hrs. The solvent was evaporated at reduced pressure, the residue partitioned between DCM (20 ml) and 0.5 M NaOH (5 ml) and the organic layer collected. The aqueous layer was re-extracted with DCM (20 ml) and the combined organic layers washed with brine (10 ml), dried (Na₂SO₄), filtered and the solvent evaporated under reduced pressure to give product (0.108 g, 98%, 0.28 mmol) as a pale yellow oil. LC/MS: 100% MH⁺, m/z 430, Rt=1.61 mins.

### General Procedure K. 4-(2-Fluoro-4-{[4-(3-fluoro-benzoyl)-piperazine-1-carbonyl]-amino}-phenyl)-piperazine-1-carboxylic acid isopropyl ester (Compound 50)

To a toluene (1 ml) solution of 4-(3-fluoro-benzoyl)-piperazine-1-carboxylic acid (3-fluoro-4-piperazin-1-yl-phenyl)-amide (1 eqv, 15 mg, 0.035 mmol) was added 1M isopropyl chloroformate in toluene (1 eqv, 0.035 ml, 0.035 mmol) and the resulting mixture stirred at room temperature. After 4 hrs the solvent was evaporated at reduced pressure and the residue partitioned between EtOAc (3 ml) and water (1 ml). The organic layer was washed with 1M K₂CO₃ aq (2×1 ml), saturated NH₄Cl aq (2×1 ml), brine (1 ml), dried (Na₂SO₄), filtered and the solvent evaporated under reduced pressure. The crude residue was purified by chromatography [SiO₂ 4 ml, eluting with EtOAc] to give product (16 mg, 91%, 0.03 mmol) as a colourless oil. LC/MS: 100% MH⁺, m/z 516, Rt=2.11 mins.

The following compound was made in a similar way as described in Route 5 Example 50 General procedure J above.

### Example 51

### Preparation of 4-(2,5-Difluoro-benzoyl)-piperazine-1-carboxylic acid (3-fluoro-4-piperazin-1-yl-phenyl)-amide (Compound 51)

In a similar fashion (Route 5: 50J, 0.18 mmol of 4-(4-{[4-(2,5-difluoro-benzoyl)-piperazine-1 -carbonyl]-amino}-2-fluoro-phenyl)-piperazine-1-carboxylic acid tert-butyl ester), gave the product (0.065 g, 81%, 0.15 mmol) as an off-white solid. LC/MS: 95% MH⁺, m/z 448, Rt=1.02 mins.

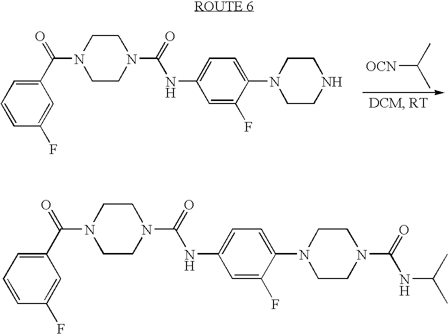

### Example 52

### 4-(3-fluorobenzoyl)-N-{3-fluoro-4-[4-(isopropylcarbamoyl)piperazin-1-yl]phenyl}piperazine-1-carboxamide (Compound 52). Potency Range B

### General Procedure L

To a DCM (1 ml) solution of 4-(3-fluoro-benzoyl)-piperazine-1-carboxylic acid (3-fluoro-4-piperazin-1-yl-phenyl)-amide (1 eqv, 15 mg, 0.035 mmol) was added a DCM (1 ml) solution of isopropyl isocyanate (1 eqv, 3 mg) and the resulting mixture stirred at room temperature for 6 hrs. The solvent was evaporated at reduced pressure and the crude material purified by chromatography [SiO₂, 4 ml, EtOAc eluent] to give pure product (11 mg, 61%, 0.02 mmol) as colourless oil. LC/MS: 100% MH⁺, m/z 515, Rt=1.85 mins.

The following compounds were made as described in Route 6 Example 52 General procedure L using intermediate compounds and commercially available isocyanates.

### Example 53

### N-{4-[4-butylcarbamoyl)piperazin-1-yl]-3-fluorophenyl}-4-(3-fluorobenzoyl)piperazine-1-carboxamide (Compound 53). Potency Range B

In a similar fashion (Route 6; 52L, 0.035 mmol 4-(3-fluoro-benzoyl)-piperazine-1-carboxylic acid (3-fluoro-4-piperazin-1-yl-phenyl)-amide), gave the product (18 mg, 97%, 0.03 mmol) as an off-white solid. LC/MS: 100% MH⁺, m/z 529, Rt=1.97 mins.

### Example 54

### N-{4-[4-(cyclopentylcarbamoyl)piperazin-1-yl]-3-fluorophenyl}-4-(3-fluorobenzoyl)piperazine-1-carboxamide (Compound 54). Potency Range B

In a similar fashion (Route 6; 52L, 0.035 mmol 4-(3-fluoro-benzoyl)-piperazine-1-carboxylic acid (3-fluoro-4-piperazin-1-yl-phenyl)-amide), gave the product (17 mg, 90%, 0.03 mmol) as an off-white solid. LC/MS: 100% MH⁺, m/z 541, Rt=1.97 mins.

### Example 55

### 4-(2,5-difluorobenzoyl)-N-{3-fluoro-4-[4-(isopropylcarbamoyl)piperazin-1-yl]phenyl}piperazine-1-carboxamide (Compound 55). Potency Range C

In a similar fashion (Route 6; 52L, 0.06 mmol 4-(2,5-difluoro-benzoyl)-piperazine-1-carboxylic acid (3-fluoro-4-piperazin-1-yl-phenyl)-amide), gave the product (13 mg, 40%, 0.02 mmol) as an off-white solid. LC/MS: 97% MH⁺, m/z 533, Rt=1.39 mins.

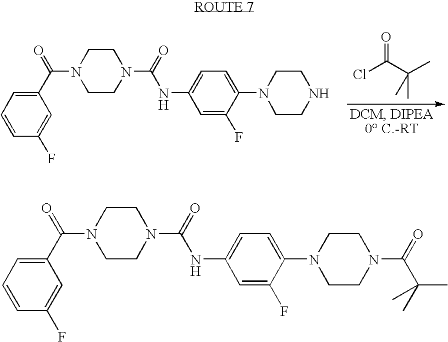

### Example 56

### Preparation of 4-(3-Fluoro-benzoyl)-piperazine-1-carboxylic acid {4-[4-(2,2-dimethyl-propionyl)-piperazin-1-yl]-3-fluoro-phenyl}-amide (Compound 56). Potency Range B

### General Procedure M

To a stirred solution of 4-(3-fluoro-benzoyl)-piperazine-1-carboxylic acid (3-fluoro-4-piperazin-1-yl-phenyl)-amide (1 eqv, 19 mg, 0.04 mmol) and DIPEA (1.5 eqv, 0.011 ml, 0.06 mmol) in DCM (1 ml) at 0° C. was added 2,2-dimethyl-propionyl chloride (1 eqv, 0.005 ml, 0.04 mmol). The mixture was warmed to room temperature and stirred for 3 hrs. The reaction mixture was diluted with DCM (2 ml) and washed with 1M K₂CO₃ aq (2 ml), saturated NH₄Cl aq (2 ml), dried (Na₂SO₄), filtered and evaporated at reduced pressure. The crude residue was purified by chromatography [SiO₂ 4 ml, eluting with EtOAc] to give product (21 mg, 93%, 0.04 mmol) as a colourless oil. LC/MS: 100% MH⁺, m/z 514, Rt=2.03 mins.

The following compounds were made as described in Route 7 Example 56 General procedure M using intermediate compounds and commercially available acid chlorides.

### Example 57

### Preparation of 4-(3-Fluoro-benzoyl)-piperazine-1-carboxylic acid [3-fluoro-4-(4-isobutyryl-piperazin-1-yl)-phenyl]-amide (Compound 57). Potency Range B

In a similar fashion (Route 7; 56M, 0.04 mmol of 4-(3-fluoro-benzoyl)-piperazine-1-carboxylic acid (3-fluoro-4-piperazin-1-yl-phenyl)-amide), gave the product (10 mg, 45%, 0.02 mmol) as a colourless oil. LC/MS: 100% MH⁺, m/z 500, Rt=1.81 mins.

### Example 58

### Preparation of 4-(2,5-Difluoro-benzoyl)-piperazine-1-carboxylic acid [3-fluoro-4-(4-isobutyryl-piperazin-1-yl)-phenyl]-amide (Compound 58). Potency Range C

In a similar fashion (Route 7: 56M, 0.06 mmol of 4-(2,5-difluoro-benzoyl)-piperazine-1-carboxylic acid (3-fluoro-4-piperazin-1-yl-phenyl)-amide), gave the product (6 mg, 17%, 0.01 mmol) as a colourless oil. LC/MS: 100% MH⁺, m/z 518, Rt=1.94 mins.

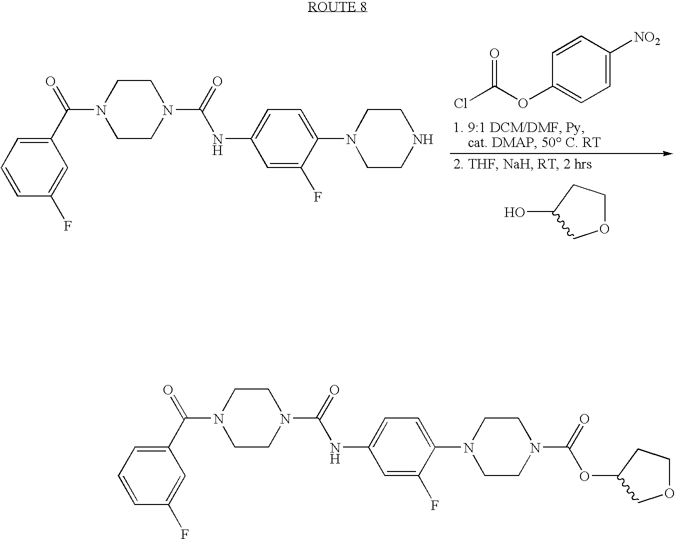

### Example 59

### 4-(2-Fluoro-4-{[4-(3-fluoro-benzoyl)-piperazine-1-carbonyl]-amino}-phenyl)-piperazine-1-carboxylic acid tetrahydro-furan-3-yl ester (Compound 59). Potency Range B

### General Procedure N

To a 9:1 DCM/DMF (2 ml) solution of 4-(3-fluoro-benzoyl)-piperazine-1-carboxylic acid (3-fluoro-4-piperazin-1-yl-phenyl)-amide (1.1 eqv, 48 mg, 0.117 mmol), pyridine (2.2 eqv, 18.0 μl, 0.223 mmol) and DMAP (0.1 eqv, 1 mg 0.01 mmol) was added 4-nitrophenyl chloroformate (1 eqv, 20.5 mg, 0.1 mmol) and the reaction mixture heated for 16 hrs at 50° C. in a sealed tube. The reaction mixture was washed with 1M HCl aq (3×3 ml), 1M KOH aq (3×3 ml), brine (2×2 ml), dried (MgSO₄), filtered and evaporated at reduced pressure to give intermediate, 4-(2-Fluoro-4-{[4-(3-fluoro-benzoyl)-piperazine-1-carbonyl]-amino}-phenyl)-piperazine-1-carboxylic acid 4-nitro-phenyl ester, as a yellow solid. A suspension of premixed tetrahydro-furan-3-ol (1 eqv, 8.9 mg, 0.101 mmol) and 60% NaH in mineral oil (1.24 eqv, 5 mg) in THF (1 ml) at RT was added to a THF (2 ml) solution of intermediate and stirred for 3 hrs at RT. The reaction mixture was diluted with EtOAc (10 ml) and washed with 1M HCl aq (2×3 ml), 1M KOH aq (2×3 ml), brine (2 ml), dried (MgSO₄), filtered and evaporated at reduced pressure to give crude product. Purification by chromatography [SiO₂, 1 ml, 99:1 DCM/MeOH eluent] gave pure racemic product (4.8 mg, 9%, 0.01 mmol) as a white solid. LC/MS: 100% MH⁺, m/z 544, Rt=1.92 mins.

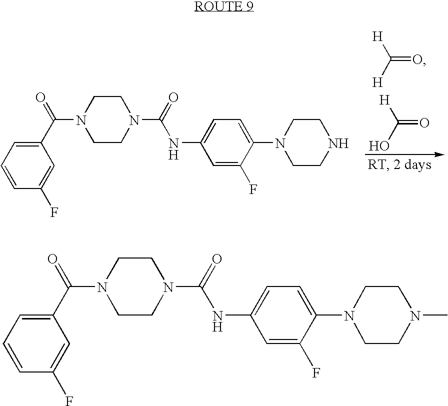

### Example 60

### 4-(3-Fluoro-benzoyl)-piperazine-1-carboxylic acid [3-fluoro-4-(4-methyl-piperazin-1-yl)-phenyl]-amide (Compound 60). Potency Range C

### General Procedure O

To a formic acid (1 ml) solution of 4-(3-fluoro-benzoyl)-piperazine-1-carboxylic acid (3-fluoro-4-piperazin-1-yl-phenyl)-amide (1 eqv, 11 mg, 0.026 mmol) was added formaldehyde (4 eqv, 10.0 μl, 0.103 mmol) and the reaction mixture stirred at RT for 2 days. The solvent was evaporated and the residue dissolved in saturated Na₂CO₃ aq (2 ml) and extracted with DCM (3×2 ml). The combined organic phase was dried (MgSO₄), filtered and evaporated at reduced pressure then purified by chromatography [SiO₂, 1 ml, EtOAc eluent] to give the product (6 mg, 52%, 0.01 mmol) as a white solid. LC/MS: 94% MH⁺, m/z 444, Rt=1.63 mins.

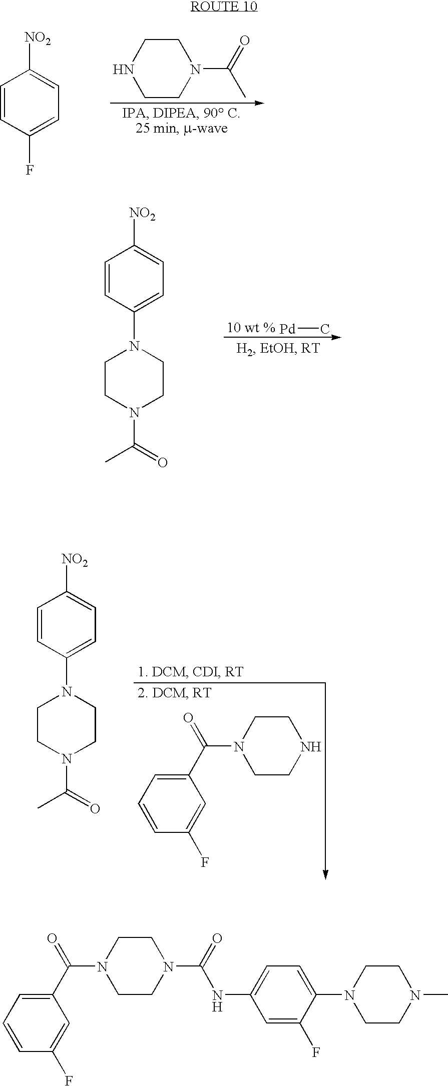

### Example 61

### 4-(3-Fluoro-benzoyl)-piperazine-1-carboxylic acid [4-(4-acetyl-piperazin-1-yl)-3-fluoro-phenyl]-amide (Compound 61). Potency Range C

### General Procedure P. 1-[4-(4-Nitro-phenyl)-piperazin-1-yl]-ethanone

A focus microwave vessel was charged with 4-fluoronitrobenzene (1 eqv, 1.41 g, 10 mmol), acetylpiperazine (1 eqv, 1.28 g, 10 mmol), DIPEA (1 eqv, 1.7 ml, 10 mmol) and propan-2-ol (2 ml). The reaction mixture was irradiated (100 W) at 90° C. for 25 mins. A yellow precipitate formed on cooling, which was collected by filtration, washed with propan-2-ol (20 ml) and dried under vacuum to give the product (1.401 g, 56%, 5.6 mmol) as a yellow solid. LC/MS: 100% MH⁺, m/z 250, Rt=1.23 mins.

### General Procedure Q. 1-[4-(4-Amino-phenyl)-piperazin-1-yl]-ethanone

To a solution of 1-[4-(4-nitro-phenyl)-piperazin-1-yl]-ethanone (1 eqv, 1.35 g, 5.42 mmol) in EtOH (50 ml) at room temperature under a nitrogen atmosphere was added palladium on carbon (10% by Wt, 0.135 g). The reaction suspension was placed under a hydrogen atmosphere (ca. 1 atm) and stirred at room temperature for 5 hrs. The black suspension was filtered through celite, to remove the palladium on carbon, and evaporated at reduced pressure to give the desired product as a white solid (1.14 g, 96%, 5.21 mmol). LC/MS: 100% MH⁺, m/z 220, Rt=0.78 mins.

### General Procedure R. 4-(3-Fluoro-benzoyl)-piperazine-1-carboxylic acid [4-(4-acetyl-piperazin-1-yl)-3-fluoro-phenyl]-amide

To a solution of 1-[4-(4-amino-phenyl)-piperazin-1-yl]-ethanone (1 eqv, 0.219 g, 1.0 mmol) in DCM (2 ml) at room temperature was added di-imidazol-1-yl-methanone (1 eqv, 0.162 mg, 1.0 mmol) and the reaction mixture stirred for 6 hrs. (3-Fluoro phenyl)-piperazin-1-yl-methanone (1.1 eqv, 0.228 g, 1.1 mmol) was added and the reaction mixture stirred at room temperature for a further 6 hrs prior to concentration at reduced pressure. The crude material was purified by preparative HPLC to give product (95 mg, 21%, 0.21 mmol) as an off-white solid. LC/MS: 99% MH⁺, m/z 454, Rt=1.62 mins.

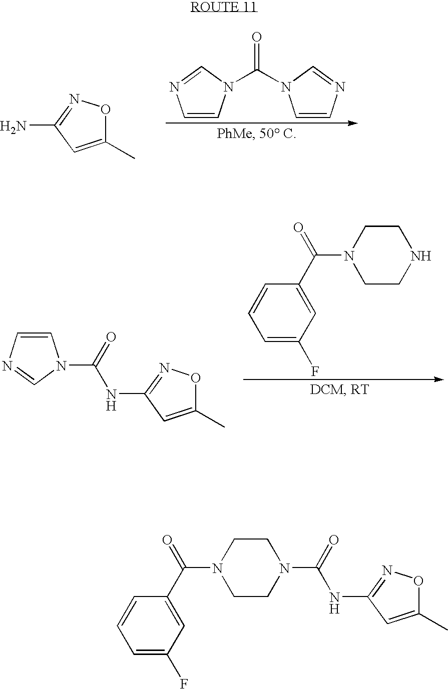

### Example 62

### Preparation of 4-(3-Fluoro-benzoyl)-piperazine-1-carboxylic acid (5-methyl-isoxazol-3-yl)-amide (Compound 62). Potency Range C

### General Procedure S. 4-(3-Fluoro-benzoyl)-piperazine-1-carboxylic acid (5-methyl-isoxazol-3-yl)-amide

A pressure tube was charged with 5-methyl-isoxazol-3-ylamine (1 eqv, 0.023 g, 0.24 mmol), di-imidazol-1-yl-methanone (1 eqv, 0.039 g, 0.24 mmol) and PhMe (1 ml) sealed and the reaction mixture stirred for between 4 and 16 hours at 50° C. then cooled to room temperature. The formation of intermediate, imidazole-1-carboxylic acid (5-methyl-isoxazol-3-yl)-amide, was monitored by LC/MS: 70% MH⁺, m/z 193, Rt=0.88 mins. To the reaction mixture was added a DCM (1 ml) solution of (3-fluoro-phenyl)-piperazin-1 yl-methanone (1.0 eqv, 0.050 g, 0.24 mmol) and the reaction mixture stirred at room temperature for 12 hours then concentrated at reduced pressure. The resulting residue was dissolved in DCM (5 ml) and washed with saturated NH₄Cl (aq) (2×3 ml), brine (2 ml), dried (Na₂SO₄), filtered and evaporated. The crude material was purified by chromatography [4 ml SiO₂, eluting with EtOAc] to give pure product (46 mg, 58%, 0.14 mmol) as a colourless oil. LC/MS: 100% MH⁺, m/z 333, Rt=1.77 mins.

The following compounds were made as described in Route 11 Example 62 General procedure S above.

### Example 63

### Preparation of 4-(3-Fluoro-benzoyl)-piperazine-1-carboxylic acid (6-chloro-pyridin-3-yl)-amide (Compound 63). Potency Range C

In a similar fashion (Route 11: 62S, 0.24 mmol of 6-chloro-pyridin-3-ylamine), gave the product (33 mg, 38%, 0.09 mmol) as a colourless oil. LC/MS: 100% MH⁺, m/z 363, Rt=1.80 mins.

### Example 64

### Preparation of 4-(3-Fluoro-benzoyl)-piperazine-1-carboxylic acid (1H-benzoimidazol-2-yl)-amide (Compound 64). Potency Range C

In a similar fashion (Route 11: 62S, 0.24 mmol of 1H-benzoimidazol-2-ylamine), gave the product (26 mg, 30%, 0.07 mmol) as a white solid. LC/MS: 100% MH⁺, m/z 368, Rt=1.47 mins.

### Example 65

### Preparation of 4-(3-Fluoro-benzoyl)-piperazine-1-carboxylic acid benzothiazol-2-ylamide (Compound 65). Potency Range B

In a similar fashion (Route 11: 62S, 0.24 mmol of benzothiazol-2-ylamine), gave the product (40 mg, 44%, 0.10 mmol) as a white solid. LC/MS: 100% MH⁺, m/z 385, Rt=2.00 mins.

### Example 66

### Preparation of 4-(3-Fluoro-benzoyl)-piperazine-1-carboxylic acid (6-fluoro-benzothiazol-2-yl)-amide (Compound 66). Potency Range B

In a similar fashion (Route 11: 62S, 0.24 mmol of 6-fluorobenzothiazol-2-ylamine), gave the product (39 mg, 40%, 0.10 mmol) as a white solid. LC/MS: 100% MH⁺, m/z 403, Rt=2.07 mins.

### Example 67

### Preparation of 4-(3-Fluoro-benzoyl)-piperazine-1-carboxylic acid (6-trifluoromethoxy-benzothiazol-2-yl)-amide (Compound 67). Potency Range B

In a similar fashion (Route 11: 62S, 0.24 mmol of 6-trifluoromethoxy-benzothiazol-2-ylamine), gave the product (30 mg, 27%, 0.06 mmol) as a white solid. LC/MS: 100% MH⁺, m/z 468, Rt=2.27 mins.

### Example 68

### Preparation of 4-(3-Fluoro-benzoyl)-piperazine-1-carboxylic acid (6-bromo-benzothiazol-2-yl)-amide (Compound 68). Potency Range A

In a similar fashion (Route 11: 62S, 0.43 mmol of 6-bromo-benzothiazol-2-ylamine), gave the product (70 mg, 35%, 0.15 mmol) as a white solid. LC/MS: 100% MH⁺, m/z 463, Rt=2.19 mins.

### Example 69

### Preparation of 4-(3-Fluoro-benzoyl)-piperazine-1-carboxylic acid (4-phenyl-thiazol-2-yl)-amide (Compound 69). Potency Range B

In a similar fashion (Route 11: 62S, 0.24 mmol of 4-phenyl-thiazol-2-ylamine), gave the product (65 mg, 66%, 0.16 mmol) as a white solid. LC/MS: 98% MH⁺, m/z 411, Rt=2.17 mins.

### Example 70

### Preparation of 4-(3-Fluoro-benzoyl)-piperazine-1-carboxylic acid (3-phenyl-[1,2,4]thiadiazol-5-yl)-amide (Compound 70). Potency Range B

In a similar fashion (Route 11: 62S, 0.24 mmol of 3-phenyl-[1,2,4]thiadiazol-5-ylamine), gave the product (18 mg, 18%, 0.04 mmol) as a white solid. LC/MS: 100% MH⁺, m/z 412, Rt=2.16 mins.

### Example 71

### Preparation of 4-(3-Fluoro-benzoyl)-piperazine-1-carboxylic acid (5-pyridin-4-yl[1,3,4]thiadiazol-2-yl)-amide (Compound 71). Potency Range B

In a similar fashion (Route 11: 62S, 0.24 mmol of 5-pyridin-4-yl[1,3,4]thiadiazol-2-ylamine), gave the product (57 mg, 58%, 0.14 mmol) as a white solid. LC/MS: 100% MH⁺, m/z 413, Rt=1.53 mins.

### Example 72

### 4-(3-Fluoro-benzoyl)-piperazine-1-carboxylic acid (5-phenyl-[1,3,4]oxadiazol-2-yl)-amide (Compound 72). Potency Range B

In a similar fashion (Route 11: 62S, 0.62 mmol of 5-phenyl-[1,3,4]oxadiazol-2-ylamine), gave the product (93 mg, 38%, 0.24 mmol) as a white solid. LC/MS: 100% MH⁺, m/z 396, Rt=1.93 mins.

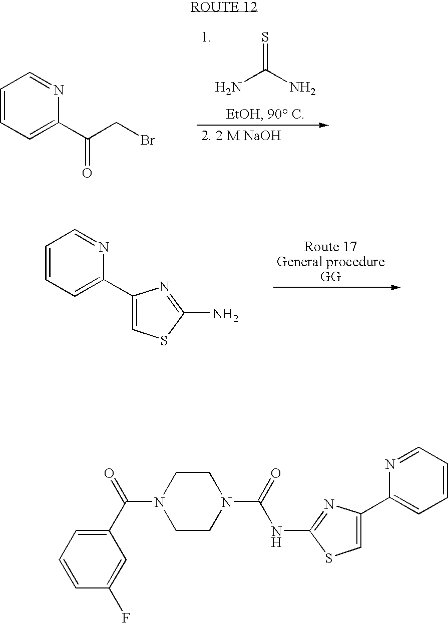

### Example 73

### 4-(3-Fluoro-benzoyl)-piperazine-1-carboxylic acid (4-pyridin-2-yl-thiazol-2-yl)-amide (Compound 73). Potency Range B

### General Procedure T. 4-Pyridin-3-yl-thiazol-2-ylamine

To a solution of 2-bromo-1-pyridin-2-yl-ethanone (1 eqv, 10 g, 50.0 mmol) in ethanol (100 ml) was added thiourea (1.02 eqv, 3.88 g, 51.0 mmol) and the reaction mixture heated to 90° C. for 2 hrs. Cooling to room temperature led to precipitation of the crude HBr product salt which was collected by filtration and washed with acetone. The solid was dissolved in 2M NaOH (25 ml), shaken for 25 mins and the basic aqueous solution extracted with EtOAc (3×30 ml). The combined organic phase was evaporated at reduced pressure to afford the product (4.83 g, 55%, 27.3 mmol) as an off-white solid. LC/MS: 100% MH⁺, m/z 178, Rt=0.64 mins.

### 4-(3-Fluoro-benzoyl)-piperazine-1-carboxylic acid (4-pyridin-2-yl-thiazol-2-yl)-amide

In a similar fashion (Route 11: 62S, 0.24 mmol of 4-pyridin-2-yl-thiazol-2-ylamine), gave the product (51 mg, 52%, 0.12 mmol) as a white solid. LC/MS: 100% MH⁺, m/z 412, Rt=1.49 mins.

The following compounds were made as illustrated in Route 12, described in Route 12 Example 73 General procedure T and Route 11 Example 62 General procedure S.

### Example 74

### 4-(3-Fluoro-benzoyl)-piperazine-1-carboxylic acid (4-pyridin-3-yl-thiazol-2-yl)-amide (Compound 74). Potency Range B

### 4-Pyridin-3-yl-thiazol-2-ylamine

Following general procedure T (to scale 30 mmol 2-bromo-1-pyridin-3-yl-ethanone) the title compound (2.023 g, 38%, 11.43 mmol) was isolated as a pale brown powder. LC/MS: 100% MH⁺, m/z 178, Rt=1.10 mins.

### 4-(3-Fluoro-benzoyl)-piperazine-1-carboxylic acid (4-pyridin-3-yl-thiazol-2-yl)-amide

In a similar fashion (Route 11: 62S, 0.24 mmol of 4-pyridin-3-yl-thiazol-2-ylamine), gave the product (39 mg, 39%, 0.09 mmol) as an off-white solid. LC/MS: 100% MH⁺, m/z 412, Rt=1.50 mins.

### Example 75

### 4-(3-Fluoro-benzoyl)-piperazine-1-carboxylic acid (4-pyridin-4-yl-thiazol-2-yl)-amide (Compound 75). Potency Range C

### 4-Pyridin-4-yl-thiazol-2-ylamine

Following general procedure T (to scale 30 mmol 2-bromo-1-pyridin-4-yl-ethanone) the title compound (0.923 g, 17%, 5.21 mmol) was isolated as an off-white powder. LC/MS: 100% MH⁺, m/z 178, Rt=1.06 mins.

### 4-(3-Fluoro-benzoyl)-piperazine-1-carboxylic acid (4-pyridin-4-yl-thiazol-2-yl)-amide

In a similar fashion (Route 11: 62S, 0.24 mmol of 4-pyridin-4-yl-thiazol-2-ylamine), gave the product (26 mg, 26%, 0.06 mmol) as an off-white solid. LC/MS: 100% MH⁺, m/z 412, Rt=1.46 mins.

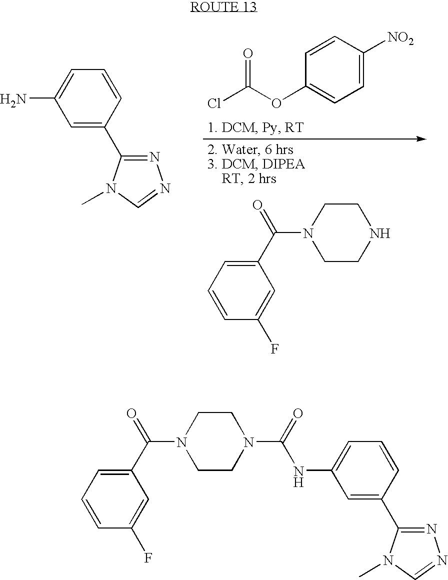

### Example 76

### 4-(3-Fluoro-benzoyl)-piperazine-1-carboxylic acid [3-(4-methyl-4H-[1,2,4]triazol-3-yl)-phenyl]-amide (Compound 76). Potency Range C

### General Procedure U

To a DCM (2 ml) solution of 3-(4-methyl-4H-[1,2,4]triazol-3-yl)-phenylamine (1 eqv, 33 mg, 0.19 mmol) and pyridine (1 eqv, 15.5 μl, 0.19 mmol) was added 4-nitrophenyl chloroformate (1.5 eqv, 58 mg, 0.29 mmol) and the reaction mixture stirred for 4 hrs at room temperature. Water (2 ml) was added to quench excess 4-nitrophenyl chloroformate and the reaction mixture stirred overnight. The aqueous layer of the resultant bi-phasic mixture was discarded. To the organic reaction mixture was added DIPEA (2 eqv, 66.9 μl, 0.35 mmol) resulting in an immediate yellow colouration. To this solution was added (3-fluoro-phenyl)-piperazin-1-yl-methanone (1 eqv, 40 mg, 0.19 mmol) in DCM (1 ml) and the reaction mixture stirred for 4 hrs at room temperature. The reaction mixture was washed with 1M HCl aq (2×3 ml), saturated Na₂CO₃ aq (2×3 ml), brine (2 ml), dried (Na₂SO₄), filtered and evaporated at reduced pressure to give crude product. Purification by preparative HPLC gave desired product (46 mg, 59%, 0.11 mmol) as a white solid. LC/MS: 100% MH⁺, m/z 409, Rt=1.64 mins.

The following compounds were made as described in Route 13 Example 76 General procedure U.

### Example 77

### 4-(3-Fluoro-benzoyl)-piperazine-1-carboxylic acid [3-(2-methyl-thiazol-4-yl)-phenyl]-amide (Compound 77). Potency Range C

In a similar fashion (Route 13: 76U, 0.19 mmol of 3-(2-methyl-thiazol-4-yl)-phenylamine), gave the product (25 mg, 31%, 0.06 mmol) as an off-white solid. LC/MS: 93% MH⁺, m/z 425, Rt=2.08 mins.

### Example 78

### 4-(3-Fluoro-benzoyl)-piperazine-1-carboxylic acid [3-(2-methyl-pyrimidin-4-yl)-phenyl]-amide (Compound 78). Potency Range B

In a similar fashion (Route 13: 76U, 0.19 mmol of 3-(2-methyl-pyrimidin-4-yl)-phenylamine), gave the product (45 mg, 56%, 0.11 mmol) as an off-white solid. LC/MS: 97% MH⁺, m/z 420, Rt=1.90 mins.

### Example 79

### 4-(3-Fluoro-benzoyl)-piperazine-1-carboxylic acid (5,6-dimethyl-benzothiazol-2-yl)-amide (Compound 79). Potency Range A

In a similar fashion (Route 13: 76U, 0.19 mmol of 5,6-dimethyl-benzothiazol-2-ylamine), gave the product (18 mg, 23%, 0.04 mmol) as a white solid. LC/MS: 100% WI⁺, m/z 413, Rt=2.19 mins.

### Example 80

### 4-(3-Fluoro-benzoyl)-piperazine-1-carboxylic acid (6-methyl-benzothiazol-2-yl)-amide (Compound 80). Potency Range A

In a similar fashion (Route 13: 76U, 0.19 mmol of 6-methyl-benzothiazol-2-ylamine), gave the product (31 mg, 41%, 0.08 mmol) as a white solid. LC/MS: 100% MH⁺, m/z 399, Rt=2.12 mins.

### Example 81

### 4-(3-Fluoro-benzoyl)-piperazine-1-carboxylic acid (6-methoxy-benzothiazol-2-yl)-amide (Compound 81). Potency Range A

In a similar fashion (Route 13: 76U, 0.19 mmol of 6-methoxy-benzothiazol-2-ylamine), gave the product (8 mg, 10%, 0.02 mmol) as a white solid. LC/MS: 100% MH⁺, m/z 415, Rt=2.07 mins.

### Example 82

### 4-(3-Fluoro-benzoyl)-piperazine-1-carboxylic acid (6-chloro-benzothiazol-2-yl)-amide (Compound 82). Potency Range A

In a similar fashion (Route 13: 76U, 0.19 mmol of 6-chloro-benzothiazol-2-ylamine), gave the product (17 mg, 21%, 0.04 mmol) as a white solid. LC/MS: 100% MH⁺, m/z 419, Rt=2.22 mins.

### Example 83

### 2-{[4-(3-Fluoro-benzoyl)-piperazine-1-carbonyl]-amino}-benzothiazole-6-carboxylic acid ethyl ester (Compound 83). Potency Range B

In a similar fashion (Route 13: 76U, 0.23 mmol of 2-amino-benzothiazole-6-carboxylic acid ethyl ester), gave the product (25 mg, 24%, 0.05 mmol) as a white solid. LC/MS: 100% MH⁺, m/z 457, Rt=2.15 mins.

### Example 84

### 4-(3-Fluoro-benzoyl)-piperazine-1-carboxylic acid (6-acetylamino-benzothiazol-2-yl)-amide (Compound 84). Potency Range B

In a similar fashion (Route 13: 76U, 0.24 mmol of N-(2-amino-benzothiazol-6-yl)-acetamide), gave the product (17 mg, 16%, 0.04 mmol) as a white solid. LC/MS: 100% MH⁺, m/z 442, Rt=1.76 mins.

### Example 85

### 4-(3-Fluoro-benzoyl)-piperazine-1-carboxylic acid (3-cyano-4-fluoro-phenyl)-amide (Compound 85). Potency Range C

In a similar fashion (Route 13: 76U, 8.83 mmol of 5-amino-2-fluoro-benzonitrile), gave the product (2.19 g, 67%, 5.92 mmol) as a white solid. LC/MS: 100% MH⁺, m/z 371, Rt=1.99 mins.

### Example 86

### 4-(3-Fluoro-benzoyl)-piperazine-1-carboxylic acid (3-pyridin-4-yl-phenyl)-amide (Compound 86). Potency Range C

In a similar fashion (Route 13: 76U, 0.17 mmol of 3-pyridin-4-yl-phenylamine), gave the product (9.6 mg, 14%, 0.02 mmol) as a white solid. LC/MS: 100% MH⁺, m/z 405, Rt=1.43 mins.

### Example 87

### 4-(3-Fluoro-benzoyl)-piperazine-1-carboxylic acid (3-pyridin-3-yl-phenyl)-amide (Compound 87). Potency Range C

In a similar fashion (Route 13: 76U, 0.17 mmol of 3-pyridin-3-yl-phenylamine), gave the product (7.6 mg, 11%, 0.02 mmol) as a white solid. LC/MS: 100% MH⁺, m/z 405, Rt=1.51 mins.

### Example 88

### 4-(3-Fluoro-benzoyl)-piperazine-1-carboxylic acid (4-bromo-3-fluoro-phenyl)-amide (Compound 88). Potency Range B

In a similar fashion (Route 13: 76U, 0.05 mmol of 4-bromo-3-fluoro-phenylamine), gave the product (6.7 mg, 33%, 0.02 mmol) as a white solid. LC/MS: 100% MH⁺, m/z 423/425, Rt=2.13 mins.

### Example 89

### 4-(3-Fluoro-benzoyl)-piperazine-1-carboxylic acid [4-(1H-pyrazol-3-yl)-phenyl]-amide (Compound 89). Potency Range B

In a similar fashion (Route 13: 76U, 0.125 mmol of 4-(1H-pyrazol-3-yl)-phenylamine), gave the product (1 mg, 2%, 0.002 mmol) as a white solid. LC/MS: 100% MH⁺, m/z 394, Rt=1.78 mins.

### Example 90

### 4-(3-Fluoro-benzoyl)-piperazine-1-carboxylic acid [4-(2-methyl-thiazol-4-yl)-phenyl]-amide (Compound 90). Potency Range B

In a similar fashion (Route 13: 76U, 0.24 mmol of 4-(2-methyl-thiazol-4-yl)-phenylamine), gave the product (19 mg, 19%, 0.05 mmol) as a white solid. LC/MS: 100% MH⁺, m/z 425, Rt=1.99 mins.

### Example 91

### 4-(3-Fluoro-benzoyl)-piperazine-1-carboxylic acid [4-(morpholine-4-sulfonyl)-phenyl]-amide (Compound 91). Potency Range B

In a similar fashion (Route 13: 76U, 0.24 mmol of 4-(morpholine-4-sulfonyl)-phenylamine), gave the product (10 mg, 9%, 0.02 mmol) as a colourless oil. LC/MS: 100% MH⁺, m/z 477, Rt=1.89 mins.

### Example 92

### 4-(3-Fluoro-benzoyl)-piperazine-1-carboxylic acid [3-(morpholine-4-sulfonyl)-phenyl]-amide (Compound 92). Potency Range C

In a similar fashion (Route 13: 76U, 0.24 mmol of 3-(morpholine-4-sulfonyl)-phenylamine), gave the product (30 mg, 26%, 0.06 mmol) as a colourless oil. LC/MS: 100% MH⁺, m/z 477, Rt=1.90 mins.

### Example 93

### 4-(3-Fluoro-benzoyl)-piperazine-1-carboxylic acid (3-methylcarbamoyl-phenyl)-amide (Compound 93). Potency Range C

In a similar fashion (Route 13: 76U, 0.24 mmol of 3-amino-N-methyl-benzamide), gave the product (41 mg, 45%, 0.10 mmol) as a colourless oil. LC/MS: 100% MH⁺, m/z 385, Rt=1.66 mins.

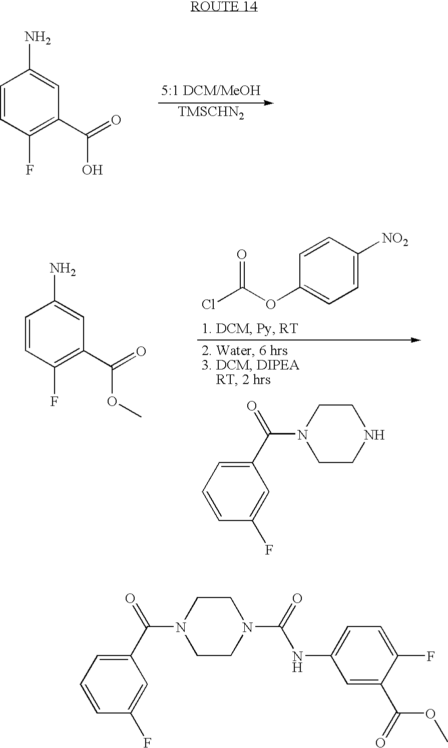

### Example 94

### 2-Fluoro-5-{[4-(3-fluoro-benzoyl)-piperazine-1-carbonyl]-amino}-benzoic acid methyl ester (Compound 94). Potency Range C

### General procedure V. 5-Amino-2-fluoro-benzoic acid methyl ester

To a stirred 5:1 DCM/MeOH (12 ml) solution of 5-amino-2-fluoro-benzoic acid (1 eqv, 2.0 g, 12.9 mmol) at 0° C. was added 2M TMSCHN₂ in hexane (1 eqv, 6.45 ml, 12.9 mmol). The reaction mixture was warmed to room temperature and stirred for 20 mins. Acetic acid (˜0.25 ml) was added dropwise until effervescence ceased. The reaction mixture concentrated at reduced pressure then dissolved in EtOAc (20 ml), washed with 2M K₂CO₃ aq (2×15 ml), brine (10 ml), dried (Na₂SO₄), filtered and evaporated at reduced pressure to give desired product (2.10 g, 96%, 12.4 mmol) as a white solid. LC/MS: 95% MH⁺, m/z 170, Rt=0.64 mins.

### 2-Fluoro-5-{[4-(3-fluoro-benzoyl)-piperazine-1-carbonyl]-amino}-benzoic acid methyl ester

In a similar fashion (Route 13: 76U, 8.93 mmol of 5-amino-2-fluoro-benzoic acid methyl ester), gave the product (2.66 g, 74%, 6.61 mmol) as an off-white solid. LC/MS: 96% MH⁺, m/z 404, Rt=1.96 mins.

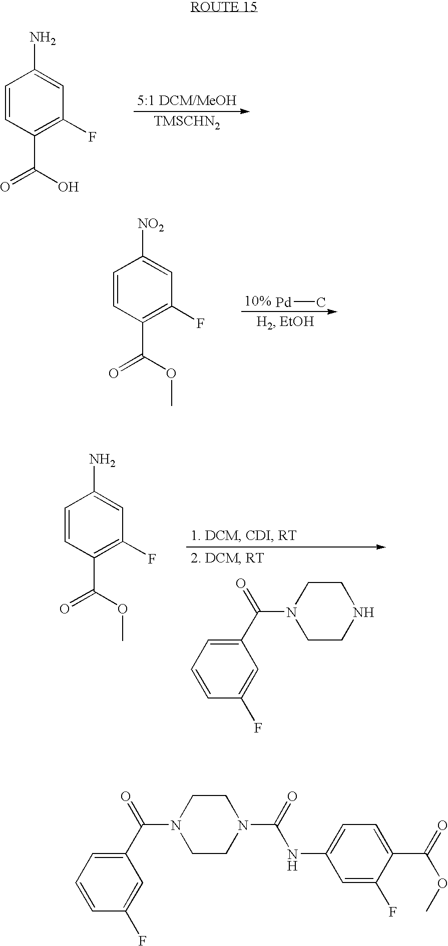

### Example 95

### 2-Fluoro-4-{[4-(3-fluoro-benzoyl)-piperazine-1-carbonyl]-amino}-benzoic acid methyl ester (Compound 95). Potency Range C

### 2-Fluoro-4-nitro-benzoic acid methyl ester

Following general procedure V (to scale 27.6 mmol of 2-fluoro-4-nitro-benzoic acid) gave the title compound (5.499 g, 100%, 27.6 mmol) as a pale yellow solid. LC/MS: 100% MH⁺, m/z 200, Rt=1.32 mins.

### 4-Amino-2-fluoro-benzoic acid methyl ester

Following general procedure Q (to scale 27.6 mmol of 2-fluoro-4-nitro-benzoic acid methyl ester) gave the title compound (4.58 g, 98%, 27.1 mmol) as an off-white solid. LC/MS: 100% MH⁺, m/z 170, Rt=1.65 mins.

### 2-Fluoro-4-{[4-(3-fluoro-benzoyl)-piperazine-1-carbonyl]-amino}-benzoic acid methyl ester

Following general procedure G using di-imidazol-1-yl-methanone (to scale 2.4 mmol of 4-amino-2-fluoro-benzoic acid methyl ester) gave the title compound (0.445 g, 46%, 1.10 mmol) as a white solid. LC/MS: 97% MH⁺, m/z 404, Rt=1.96 mins.

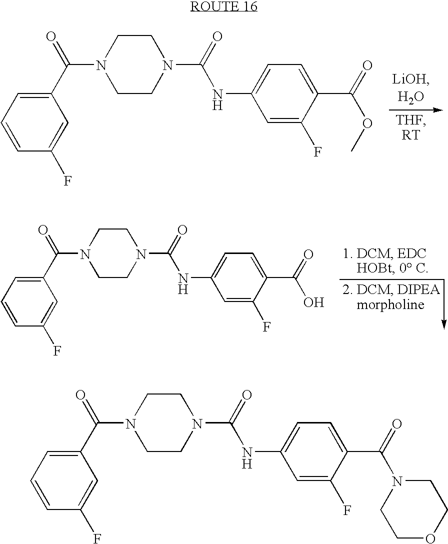

### Example 96

### 2-Fluoro-4-{[4-(3-fluoro-benzoyl)-piperazine-1-carbonyl]-amino}-benzoic acid (Compound 96). Potency Range C

### General Procedure W

To a stirred 10:3 water/THF (13 ml) solution of 2-fluoro-4-{[4-(3-fluoro-benzoyl)-piperazine-1-carbonyl]-amino}-benzoic acid methyl ester (1 eqv, 0.39 g, 0.84 mmol) at room temperature was added LiOH (3 eqv, 0.105 g, 2.51 mmol). The reaction mixture was stirred for 5 hrs then acidified to pH 3.5 with 1M HCl, and extracted into EtOAc (2×25 ml). The combined organic phase was washed with brine (2×15 ml), dried (Na₂SO₄), filtered and evaporated at reduced pressure to give product (0.290 g, 89%, 0.745 mmol) as an off-white solid. LC/MS: 97% MH⁺, m/z 390, Rt=1.78 mins.

### Example 97

### 4-(3-Fluoro-benzoyl)-piperazine-1-carboxylic acid [3-fluoro-4-(morpholine-4-carbonyl)-phenyl]-amide (Compound 97). Potency Range C

### General Procedure X

To a stirred solution of 2-fluoro-4-{[4-(3-fluoro-benzoyl)-piperazine-1-carbonyl]-amino}-benzoic acid (1 eqv, 60 mg, 0.15 mmol), EDC (1 eqv, 29 mg, 0.15 mmol) and HOBt (0.2 eqv, 4 mg, 0.03 mmol) in DCM (2 ml) at 0° C. was added a DCM (1 ml) solution of morpholine (1.1 eqv, 14.7 mg, 0.17 mmol) and DIPEA (2.2 eqv, 58.9 μl, 0.34 mmol). The reaction mixture was warmed to room temperature and stirred for 12 hrs, then concentrated at reduced pressure. The crude residue was dissolved in EtOAc (4 ml) and water (3 ml). The organic layer was washed with 10% citric acid aq (2×3 ml), 1M K₂CO₃ (2×3 ml), brine (3 m), dried (Na₂SO₄), filtered and evaporated at reduced pressure. Purification by chromatography [SiO₂, 7 ml, eluting with EtOAc] gave product (30 mg, 38%, 0.06 mmol) as colourless oil. LC/MS: 100% MH⁺, m/z 458, Rt=1.73 mins.

The following compounds were made as described in Route 16 Example 97 General procedure X.

### Example 98

### 4-(3-Fluoro-benzoyl)-piperazine-1-carboxylic acid [3-(2-methyl-thiazol-4-yl)-phenyl]amide (Compound 98). Potency Range B

In a similar fashion (Route 16: 97X, 0.15 mmol of 2-fluoro-4-{[4-(3-fluoro-benzoyl)-piperazine-1-carbonyl]-amino}-benzoic acid) gave the product (28 mg, 38%, 0.06 mmol) as a colourless oil. LC/MS: 100% MH⁺, m/z 444, Rt=1.91 mins.

### Example 99

### 4-(3-Fluoro-benzoyl)-piperazine-1-carboxylic acid [3-fluoro-4-(6-fluoro-pyridin-3-ylcarbamoyl)-phenyl]-amide (Compound 99). Potency Range C

In a similar fashion (Route 16: 97X, 0.15 mmol of 2-fluoro-4-{[4-(3-fluoro-benzoyl)-piperazine-1-carbonyl]-amino}-benzoic acid) gave the product (25 mg, 31%, 0.05 mmol) as a white solid. LC/MS: 100% MH⁺, m/z 484, Rt=1.85 mins.

The following compound was made as described in Route 16 Example 96 General procedure W

### Example 100

### 4-{[4-(3-Fluoro-benzoyl)-piperazine-1-carbonyl]-amino}-benzoic acid (Compound 100). Potency Range C

In a similar fashion (Route 16: 96W, 0.88 mmol of 4-{[4-(3-fluoro-benzoyl)-piperazine-1-carbonyl]-amino}-benzoic acid ethyl ester) gave the product (124 mg, 38%, 0.33 mmol) as an off-white solid. LC/MS: 100% MH⁺, m/z 372, Rt=1.66 mins.

The following compounds were made as described in Route 16 Example 97 General procedure X

### Example 101

### 4-(3-Fluoro-benzoyl)-piperazine-1-carboxylic acid [4-(morpholine-4-carbonyl)-phenyl]-amide (Compound 101). Potency Range C

In a similar fashion (Route 16: 97X, 0.05 mmol of 4-{[4-(3-fluoro-benzoyl)-piperazine-1-carbonyl]-amino}-benzoic acid) gave the product (6.4 mg, 27%, 0.01 mmol) as an off-white solid. LC/MS: 100% MH⁺, m/z 441, Rt=1.63 mins.

### Example 102

### 4-(3-Fluoro-benzoyl)-piperazine-1-carboxylic acid (4-diethylcarbamoyl-phenyl)-amide (Compound 102). Potency Range C

In a similar fashion (Route 16: 97X, 0.05 mmol of 4-{[4-(3-fluoro-benzoyl)-piperazine-1-carbonyl]-amino}-benzoic acid) gave the product (6.9 mg, 30%, 0.02 mmol) as an off-white solid. LC/MS: 90% MH⁺, m/z 427, Rt=1.75 mins.

### Example 103

### 4-(3-Fluoro-benzoyl)-piperazine-1-carboxylic acid [4-(6-fluoro-pyridin-3-ylcarbamoyl)-phenyl]-amide (Compound 103). Potency Range C

In a similar fashion (Route 16: 97X, 0.05 mmol of 4-{[4-(3-fluoro-benzoyl)-piperazine-1-carbonyl]-amino}-benzoic acid) gave the product (4 mg, 17%, 0.01 mmol) as an off-white solid. LC/MS: 100% MH⁺, m/z 466, Rt=1.88 mins.

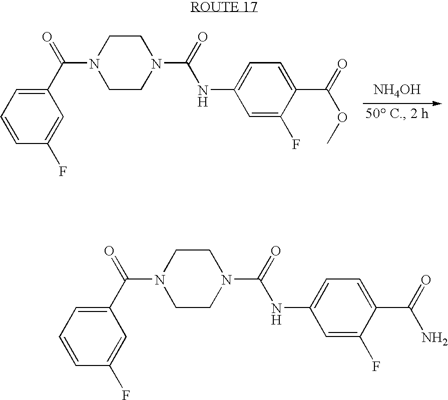

### Example 104

### 4-(3-Fluoro-benzoyl)-piperazine-1-carboxylic acid (4-carbamoyl-3-fluoro-phenyl)-amide (Compound 104). Potency Range C

### General Procedure Y

A pressure tube was charged with 2-fluoro-4-{[4-(3-fluoro-benzoyl)-piperazine-1-carbonyl]-amino}-benzoic acid methyl ester (1 eqv, 79 mg, 0.196 mmol) and 28% NH₄OH aq (2 ml), sealed and heated to 50° C. for 2 hrs. On cooling to room temperature precipitation was noted. The solid was collected by filtration and washed with water (2×2 ml) and dried at reduced pressure to give product (16 mg, 21%, 0.04 mmol) as a white solid. LC/MS: 97% MH⁺, m/z 389, Rt=1.68 mins.

The following compound was made as described in Route 17 Example 104 General procedure Y

### Example 105

### 4-(3-Fluoro-benzoyl)-piperazine-1-carboxylic acid (4-carbamoyl-phenyl)-amide (Compound 105). Potency Range C

In a similar fashion (Route 17: 104Y, 0.31 mmol of 4-{[4-(3-fluoro-benzoyl)-piperazine-1-carbonyl]-amino}-benzoic acid ethyl ester) gave the product (35 mg, 31%, 0.09 mmol) as an off-white solid. LC/MS: 100% MH⁺, m/z 371, Rt=1.60 mins.

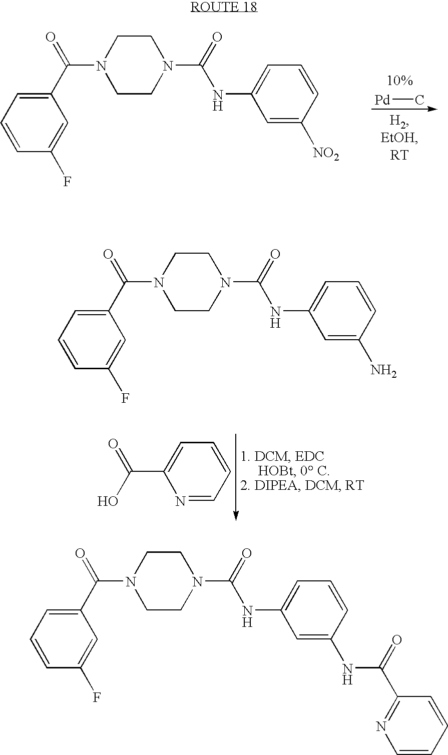

### Example 106

### 4-(3-Fluoro-benzoyl)-piperazine-1-carboxylic acid (3-amino-phenyl)-amide (Compound 106). Potency Range C

Following general procedure Q (to scale 1.73 mmol of 4-(3-fluoro-benzoyl)-piperazine-1-carboxylic acid (4-nitro-phenyl)-amide) gave the title compound (0.278 g, 47%, 0.82 mmol) as a white solid. LC/MS: 100% MH⁺, m/z 342, Rt=1.35 mins.

The following compounds were made as illustrated in Route 18, as described in Route 16 Example 97 General procedure X

### Example 107

### 4-(3-Fluoro-benzoyl)-piperazine-1-carboxylic acid {3-[(pyridine-2-carbonyl)-amino]-phenyl}-amide (Compound 107). Potency Range B

In a similar fashion (Route 16: 97X, 0.1 mmol of 4-(3-fluoro-benzoyl)-piperazine-1-carboxylic acid (3-amino-phenyl)-amide) gave the title compound (20 mg, 43%, 0.04 mmol) as a white solid. LC/MS: 100% MH⁺, m/z 448, Rt=1.97 mins.

### Example 108

### 4-(3-Fluoro-benzoyl)-piperazine-1-carboxylic acid {3-[(1-oxy-pyridine-2-carbonyl)-amino]-phenyl}-amide (Compound 108). Potency Range C

In a similar fashion (Route 16: 97X, 0.1 mmol of 4-(3-fluoro-benzoyl)-piperazine-1-carboxylic acid (3-amino-phenyl)-amide) gave the title compound (9 mg, 19%, 0.02 mmol) as a white solid. LC/MS: 100% MH⁺, m/z 464, Rt=1.80 mins.

### Example 109

### 4-(3-Fluoro-benzoyl)-piperazine-1-carboxylic acid {3-[(1-oxy-pyridine-3-carbonyl)-amino]-phenyl}-amide (Compound 109). Potency Range C

In a similar fashion (Route 16: 97X, 0.1 mmol of 4-(3-fluoro-benzoyl)-piperazine-1-carboxylic acid (3-amino-phenyl)-amide) gave the title compound (25 mg, 53%, 0.05 mmol) as a white solid. LC/MS: 100% MH⁺, m/z 464, Rt=1.61 mins.

### Example 110

### 4-(3-Fluoro-benzoyl)-piperazine-1-carboxylic acid {3-[(1-oxy-pyridine-4-carbonyl)-amino]-phenyl}-amide (Compound 110). Potency Range C

In a similar fashion (Route 16: 97X, 0.1 mmol of 4-(3-fluoro-benzoyl)-piperazine-1-carboxylic acid (3-amino-phenyl)-amide) gave the title compound (3 mg, 7%, 0.01 mmol) as a white solid. LC/MS: 100% MH⁺, m/z 464, Rt=1.61 mins.

### Example 111

### 4-(3-Fluoro-benzoyl)-piperazine-1-carboxylic acid {3-[(6-fluoro-pyridine-3-carbonyl)-amino]-phenyl}-amide (Compound 111). Potency Range C

In a similar fashion (Route 16: 97X, 0.1 mmol of 4-(3-fluoro-benzoyl)-piperazine-1-carboxylic acid (3-amino-phenyl)-amide) gave the title compound (15 mg, 32%, 0.03 mmol) as a white solid. LC/MS: 100% MH⁺, m/z 465, Rt=1.88 mins.

The following compounds were made as illustrated in Route 18, but described in Route 10 Example 61 General procedure Q

### Example 112

### 4-(3-Fluoro-benzoyl)-piperazine-1-carboxylic acid (4-amino-phenyl)-amide (Compound 112). Potency Range C

In a similar fashion (Route 10: 61Q, 1.17 mmol of 4-(3-fluoro-benzoyl)-piperazine-1-carboxylic acid (4-nitro-phenyl)-amide) gave the title compound (400 mg, 100%, 1.17 mmol) as an off-white solid. LC/MS: 100% MH⁺, m/z 343, Rt=0.98 mins.

### Example 113

### 4-(2,5-Difluoro-benzoyl)-piperazine-1-carboxylic acid (4-amino-phenyl)-amide (Compound 113). Potency Range C

In a similar fashion (Route 10: 61Q, 1.77 mmol of 4-(2,5-di-fluoro-benzoyl)-piperazine-1-carboxylic acid (4-nitro-phenyl)-amide) gave the title compound (561 mg, 88%, 1.56 mmol) as an off-white solid. LC/MS: 98% MH⁺, m/z 361, Rt=1.30 mins.

The following compounds were made as illustrated in Route 18, but described in Route 16 Example 97 General procedure X

### Example 114

### 4-(3-Fluoro-benzoyl)-piperazine-1-carboxylic acid {4-[(pyridine-2-carbonyl)-amino]-phenyl}-amide (Compound 114). Potency Range B

In a similar fashion (Route 16: 97X, 0.1 mmol of 4-(3-fluoro-benzoyl)-piperazine-1-carboxylic acid (4-amino-phenyl)-amide) gave the title compound (4.3 mg, 10%, 0.01 mmol) as a white solid. LC/MS: 100% MH⁺, m/z 448, Rt=1.94 mins.

### Example 115

### 4-(3-Fluoro-benzoyl)-piperazine-1-carboxylic acid {4-[(1-oxy-pyridine-2-carbonyl)-amino]-phenyl}-amide (Compound 115). Potency Range B

In a similar fashion (Route 16: 97X, 0.1 mmol of 4-(3-fluoro-benzoyl)-piperazine-1-carboxylic acid (4-amino-phenyl)-amide) gave the title compound (4.6 mg, 10%, 0.01 mmol) as an off-white solid. LC/MS: 100% MH⁺, m/z 464, Rt=1.77 mins.

### Example 116

### 4-(3-Fluoro-benzoyl)-piperazine-1-carboxylic acid {4-[(1-oxy-pyridine-3-carbonyl)-amino]-phenyl}-amide (Compound 116). Potency Range B

In a similar fashion (Route 16: 97X, 0.1 mmol of 4-(3-fluoro-benzoyl)-piperazine-1-carboxylic acid (4-amino-phenyl)-amide) gave the title compound (4.6 mg, 10%, 0.01 mmol) as an off-white solid. LC/MS: 100% MH⁺, m/z 464, Rt=1.61 mins.

### Example 117

### 4-(3-Fluoro-benzoyl)-piperazine-1-carboxylic acid {4-[(6-fluoro-pyridine-3-carbonyl)-amino]-phenyl}-amide (Compound 117). Potency Range C

In a similar fashion (Route 16: 97X, 0.1 mmol of 4-(3-fluoro-benzoyl)-piperazine-1-carboxylic acid (4-amino-phenyl)-amide) gave the title compound (5.4 mg, 12%, 0.01 mmol) as an off-white solid. LC/MS: 98% MH⁺, m/z 466, Rt=1.84 mins.

### Example 118

### 4-(2,5-Difluoro-benzoyl)-piperazine-1-carboxylic acid {4-[(1-oxy-pyridine-2-carbonyl)-amino]-phenyl}-amide (Compound 118). Potency Range B

In a similar fashion (Route 16: 97X, 0.11 mmol of 4-(2,5-di-fluoro-benzoyl)-piperazine-1-carboxylic acid (4-amino-phenyl)-amide) gave the title compound (7 mg, 13%, 0.01 mmol) as an off-white solid after purification by preparative HPLC. LC/MS: 98% MH⁺, m/z 482, Rt=1.77 mins.

### Example 119

### 4-(2,5-Difluoro-benzoyl)-piperazine-1-carboxylic acid {4-[(1-oxy-pyridine-3-carbonyl)-amino]-phenyl}-amide (Compound 119). Potency Range B

In a similar fashion (Route 16: 97X, 0.11 mmol of 4-(2,5-di-fluoro-benzoyl)-piperazine-1-carboxylic acid (4-amino-phenyl)-amide) gave the title compound (3 mg, 6%, 0.01 mmol) as an off-white solid after purification by preparative HPLC. LC/MS: 96% MH⁺, m/z 482, Rt=1.58 mins.

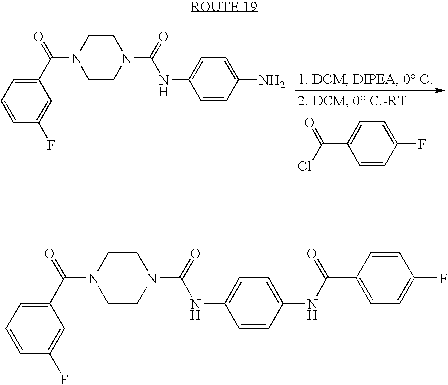

The following compounds were made as illustrated in Route 19, but described in Route 7 Example 56 General procedure M using intermediate compounds and commercially available acid chlorides.

### Example 120

### 4-(3-Fluoro-benzoyl)-piperazine-1-carboxylic acid [4-(4-fluoro-benzoylamino)-phenyl]-amide (Compound 120). Potency Range B

In a similar fashion (Route 7: 56M, 0.15 mmol of 4-(3-fluoro-benzoyl)-piperazine-1-carboxylic acid (4-amino-phenyl)-amide), gave the product (28 mg, 40%, 0.06 mmol) as a colourless oil. LC/MS: 97% MH⁺, m/z 465, Rt=1.87 mins.

### Example 121

### 4-(3-Fluoro-benzoyl)-piperazine-1-carboxylic acid (4-isobutyrylamino-phenyl)-amide (Compound 121). Potency Range C

In a similar fashion (Route 7: 56M, 0.15 mmol of 4-(3-fluoro-benzoyl)-piperazine-1-carboxylic acid (4-amino-phenyl)-amide), gave the product (28 mg, 45%; 0.07 mmol) as a colourless oil. LC/MS: 100% MH⁺, m/z 413, Rt=1.73 mins.

### Example 122

### 4-(3-Fluoro-benzoyl)-piperazine-1-carboxylic acid {4-[(pyridine-3-carbonyl)-amino]-phenyl}-amide (Compound 122). Potency Range C

In a similar fashion (Route 7: 56M, 0.10 mmol of 4-(3-fluoro-benzoyl)-piperazine-1-carboxylic acid (4-amino-phenyl)-amide), gave the product (3 mg, 7%, 0.01 mmol) as a white solid. LC/MS: 100% MH⁺, m/z 448, Rt=1.64 mins.

### Example 123

### 4-(3-Fluoro-benzoyl)-piperazine-1-carboxylic acid {4-[(pyridine-4-carbonyl)-amino]-phenyl}-amide (Compound 123). Potency Range C

In a similar fashion (Route 7: 56M, 0.10 mmol of 4-(3-fluoro-benzoyl)-piperazine-1-carboxylic acid (4-amino-phenyl)-amide), gave the product (7 mg, 15%, 0.02 mmol) as a white solid. LC/MS: 100% MH⁺, m/z 448, Rt=1.61 mins.

### Example 124

### 4-(3-Fluoro-benzoyl)-piperazine-1-carboxylic acid {4-[(tetrahydro-pyran-4-carbonyl)-amino]-phenyl}-amide (Compound 124). Potency Range C

In a similar fashion (Route 7: 56M, 0.10 mmol of 4-(3-fluoro-benzoyl)-piperazine-1-carboxylic acid (4-amino-phenyl)-amide), gave the product (4 mg, 8%, 0.01 mmol) as a white solid. LC/MS: 98% MH⁺, m/z 455, Rt=1.70 mins.

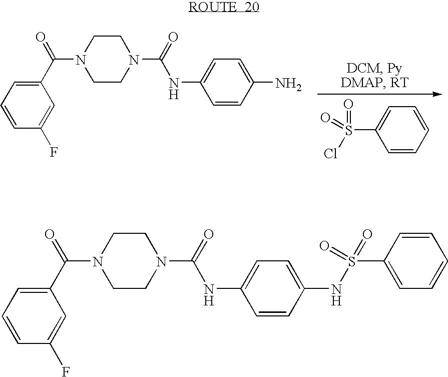

### Example 125

### 4-(3-Fluoro-benzoyl)-piperazine-1-carboxylic acid (4-benzenesulfonylamino-phenyl)-amide (Compound 125). Potency Range B

### General Procedure Z

To a DCM (1 ml) solution of 4-(3-fluoro-benzoyl)-piperazine-1-carboxylic acid (4-amino-phenyl)-amide (1 eqv, 50 mg, 0.15 mmol), pyridine (5 eqv, 59 μl, 0.73 mmol) and DMAP (0.1 eqv, 2 mg, 0.015 mmol) was added benzenesulfonyl chloride (1.05 eqv, 19.5 ml, 0.16 mmol) and the reaction mixture stirred at room temperature for 12 hrs. The reaction mixture was diluted by addition of DCM (2 ml), washed with saturated NH₄Cl aq (2×2 ml), 1M Na₂CO₃ aq (2×2 ml) and brine (2 ml), dried (MgSO₄), filtered and evaporated at reduced pressure to give crude material. Purification by chromatography [SiO₂ 4 ml, eluting with DCM, then 1:1 DCM/EtOAc through to EtOAc] gave product (25 mg, 35%, 0.05 mmol) as a white solid. LC/MS: 100% MH⁺, m/z 483, Rt=1.87 mins.

The following compounds were made as described in Route 20 Example 125 General procedure Z

### Example 126

### 4-(3-Fluoro-benzoyl)-piperazine-1-carboxylic acid (4-methanesulfonylamino-phenyl)-amide (Compound 126). Potency Range C

In a similar fashion (Route 20: 125Z, 0.15 mmol of 4-(3-fluoro-benzoyl)-piperazine-1-carboxylic acid (4-amino-phenyl)-amide), gave the product (29 mg, 46%, 0.07 mmol) as a white solid. LC/MS: 100% MH⁺, m/z 421, Rt=1.68 mins.

### Example 127

### 4-(3-Fluoro-benzoyl)-piperazine-1-carboxylic acid (4-cyclopropanesulfonylamino-phenyl)-amide (Compound 127). Potency Range B

In a similar fashion (Route 20: 125Z, 0.10 mmol of 4-(3-fluoro-benzoyl)-piperazine-1-carboxylic acid (4-amino-phenyl)-amide), gave the product (7 mg, 16%, 0.02 mmol) as a white solid. LC/MS: 100% MH⁺, m/z 447, Rt=1.81 mins.

### Example 128

### 4-(3-Fluoro-benzoyl)-piperazine-1-carboxylic acid [4-(4-fluoro-benzenesulfonylamino)-phenyl]-amide (Compound 128). Potency Range B

In a similar fashion (Route 20: 125Z, 0.10 mmol of 4-(3-fluoro-benzoyl)-piperazine-1-carboxylic acid (4-amino-phenyl)-amide), gave the product (7 mg, 14%, 0.01 mmol) as a white solid. LC/MS: 100% MH⁺, m/z 501, Rt=2.01 mins.

### Example 129

### N-{4-[bis(methylsulfonyl)amino]phenyl}-4-(3-fluorobenzoyl)piperazine-1-carboxamide (Compound 129). Potency Range B

In a similar fashion (Route 20: 125Z, 0.15 mmol of 4-(3-fluoro-benzoyl)-piperazine-1-carboxylic acid (4-amino-phenyl)-amide), gave the bis-capped product (15 mg, 33%, 0.03 mmol) as a white solid. LC/MS: 100% MH⁺, m/z 499, Rt=1.87 mins.

### Example 130

### 4-(3-Fluoro-benzoyl)-piperazine-1-carboxylic acid [3-(4-fluoro-benzenesulfonylamino)-phenyl]-amide (Compound 130). Potency Range C

In a similar fashion (Route 20: 125Z, 0.10 mmol of 4-(3-fluoro-benzoyl)-piperazine-1-carboxylic acid (3-amino-phenyl)-amide), gave the product (1.4 mg, 3%, 0.01 mmol) as an off-white solid. LC/MS: 96% MH⁺, m/z 501, Rt=2.05 mins.

### Example 131

### 4-(3-Fluoro-benzoyl)-piperazine-1-carboxylic acid (3-cyclopropanesulfonylamino-phenyl)-amide (Compound 131). Potency Range C

In a similar fashion (Route 20: 125Z, 0.10 mmol of 4-(3-fluoro-benzoyl)-piperazine-1-carboxylic acid (3-amino-phenyl)-amide), gave the product (24 mg, 53%, 0.05 mmol) as an off-white solid. LC/MS: 100% MH⁺, m/z 447, Rt=1.85 mins.

### Example 132

### 4-(2,5-Difluoro-benzoyl)-piperazine-1-carboxylic acid (4-cyclopropanesulfonylamino-phenyl)-amide (Compound 132). Potency Range C

In a similar fashion (Route 20: 125Z, 0.10 mmol of 4-(2,5-difluoro-benzoyl)-piperazine-1-carboxylic acid (4-amino-phenyl)-amide), gave the product (6 mg, 13%, 0.01 mmol) as a white solid. LC/MS: 100% MH⁺, m/z 465, Rt=1.83 mins.

### Example 133

### 4-(2,5-Difluoro-benzoyl)-piperazine-1-carboxylic acid (4-methanesulfonylamino-phenyl)-amide (Compound 133). Potency Range C

In a similar fashion (Route 20: 125Z, 0.10 mmol of 4-(2,5-difluoro-benzoyl)-piperazine-1-carboxylic acid (4-amino-phenyl)-amide), gave the product (12 mg, 27%, 0 .03 mmol) as a white solid. LC/MS: 97% MH⁺, m/z 439, Rt=1.79 mins.

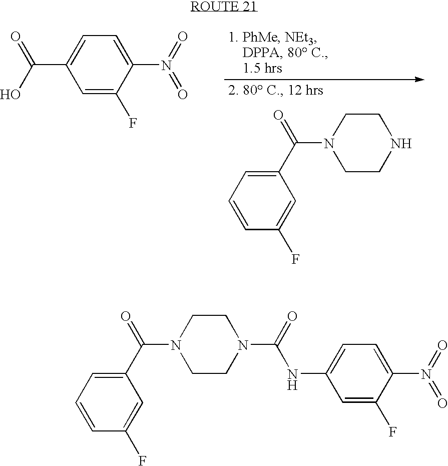

### Example 134

### 4-(3-Fluoro-benzoyl)-piperazine-1-carboxylic acid (3-fluoro-4-nitro-phenyl)-amide (Compound 134). Potency Range C

### General Procedure AA

A pressure tube was charged with 3-fluoro-4-nitro-benzoic acid (1 eqv, 50 mg, 0.27 mmol), NEt₃ (1 eqv, 38 μl, 0.27 mmol), DPPA (1 eqv, 58 μl, 0.27 mmol) and toluene (2 ml), sealed and heated to 80° C. for 1.5 hrs. Progress of the reaction was monitored by removal of an aliquot, quench with MeOH and LCMS. After consumption of the carboxylic acid starting material (3-fluoro-phenyl)-piperazin-1-yl-methanone (1 eqv, 56 mg, 0.27 mmol) was added and the reaction mixture heated to 80° C. for 14 hours. The reaction mixture was diluted with EtOAc (5 ml) and washed with 10% citric acid aq (2×3 ml), 2M K₂CO₃ aq (2×3 ml) and brine (2 ml), dried (MgSO₄), filtered and evaporated at reduced pressure to give crude material. Purification by chromatography [SiO₂ 8 ml, eluting with DCM through to 95:5 DCM/MeOH] gave product (2.5 mg, 2%, 0.01 mmol) as colourless oil. LC/MS: 100% MH⁺, m/z 391, Rt=2.05 mins.

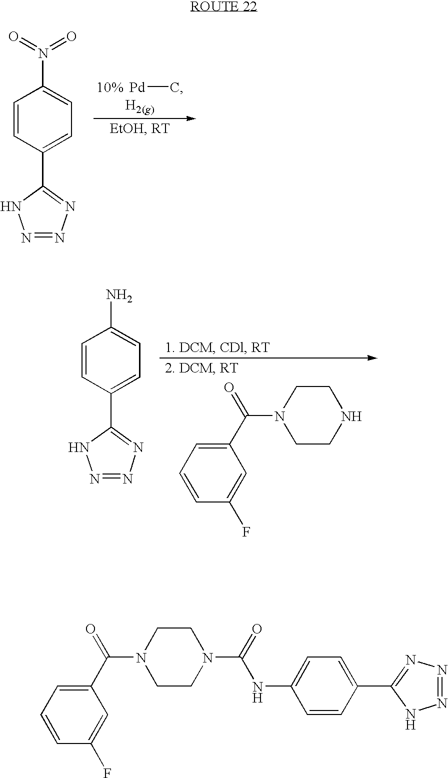

### Example 135

### 4-(3-Fluoro-benzoyl)-piperazine-1-carboxylic acid [4-(1H-tetrazol-5-yl)-phenyl]-amide (Compound 135). Potency Range B

### 4-(1H-Tetrazol-5-yl)-phenylamine

Following general procedure Q (to scale 2.62 mmol of 5-(4-nitro-phenyl)-1H-tetrazole) gave the title compound (0.204 g, 48%, 1.27 mmol) as a white solid. LC/MS: 94% MH⁺, m/z 162, Rt=1.11 mins.

### 2-Fluoro-4-{[4-(3-fluoro-benzoyl)-piperazine-1-carbonyl]-amino}-benzoic acid methyl ester

Following general procedure G using di-imidazol-1-yl-methanone (to scale 2.4 mmol of 4-(1H-tetrazol-5-yl)-phenylamine) gave the title compound (2 mg, 2%, 0.004 mmol) as colourless oil. LC/MS: 95% m/z 396, Rt=1.70 mins.

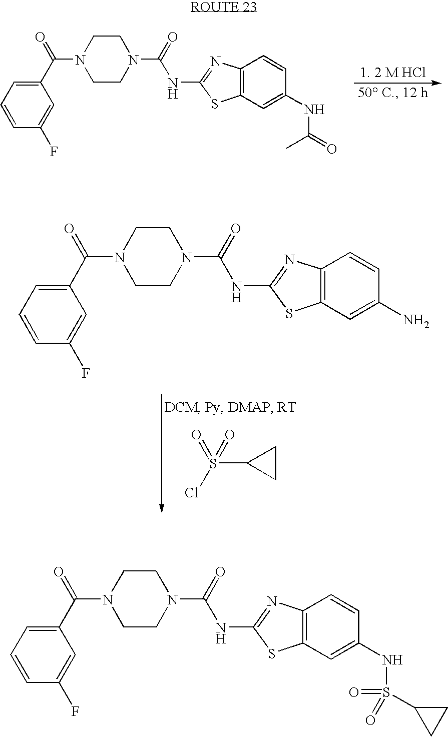

### Example 136

### 4-(3-Fluoro-benzoyl)-piperazine-1-carboxylic acid (6-amino-benzothiazol-2-yl)-amide (Compound 136). Potency Range B

### General Procedure BB

A pressure tube was charged with a solution of 4-(3-fluoro-benzoyl)-piperazine-1-carboxylic acid (6-acetylamino-benzothiazol-2-yl)-amide (1 eqv, 0.08 g, 0.018 mmol) in 1.2M HCl aq (1 ml), sealed and the stirred reaction mixture heated to 50° C. for 12 hours. The solvent was evaporated at reduced pressure to give product (12 mg, 95%, 0.03 mmol) as a white solid. LC/MS: 91% MH⁺, m/z 399, Rt=1.44 mins.

The following compound was made as illustrated in Route 23, as described in Route 20 Example 125 General procedure Z

### Example 137

### 4-(3-Fluoro-benzoyl)-piperazine-1-carboxylic acid (6-cyclopropanesulfonylamino-benzothiazol-2-yl)-amide (Compound 137). Potency Range B

In a similar fashion (Route 20: 125Z, 0.028 mmol of 4-(3-fluoro-benzoyl)-piperazine-1-carboxylic acid (6-amino-benzothiazol-2-yl)-amide), gave the product (0.8 mg, 6%, 0.002 mmol) as an off-white solid. LC/MS: 85% MH⁺, m/z 504, Rt=1.90 mins.

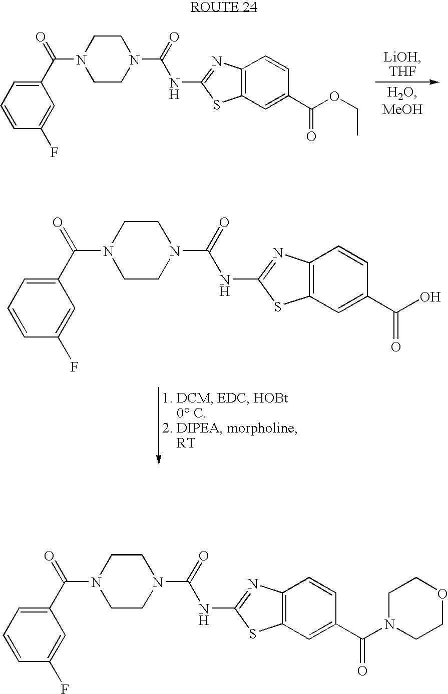

### Example 138

### 2-{[4-(3-Fluoro-benzoyl)-piperazine-1-carbonyl]-amino}-benzothiazole-6-carboxylic acid (Compound 138). Potency Range B

### General Procedure CC

To a stirred 3:2 THF/MeOH solution of 2-{[4-(3-fluoro-benzoyl)-piperazine-1-carbonyl]-amino}-benzothiazole-6-carboxylic acid ethyl ester (1 eqv, 0.400 g, 0.876 mmol) at room temperature was added LiOH (6 eqv, 0.126 g, 5.26 mmol). Upon consumption of ester starting material, as shown by TLC, the solvent was evaporated and the resulting residue dissolved in 3:2 water/THF (10 ml). The reaction mixture was stirred for 12 hrs then acidified to pH 3.5 with 1M HCl, and extracted into EtOAc (2×25 ml). The combined organic phase was washed with brine (2×15 ml), dried (Na₂SO₄), filtered and evaporated at reduced pressure to give product (0.374 g, 99%, 0.87 mmol) as an off-white solid. LC/MS: 97% MH⁺, m/z 429, Rt=1.74 mins.

The following compound was made as illustrated in Route 24, as described in Route 16 Example 97 General procedure X

### Example 139

### 4-(3-Fluoro-benzoyl)-piperazine-1-carboxylic acid [6-(morpholine-4-carbonyl)-benzothiazol-2-yl]-amide (Compound 139). Potency Range B

In a similar fashion (Route 16: 97X, 0.23 mmol of 2-{[4-(3-fluoro-benzoyl)-piperazine-1-carbonyl]-amino}-benzothiazole-6-carboxylic acid), gave the product (6 mg, 5%, 0.01 mmol) as a colourless oil. LC/MS: 100% MH⁺, m/z 498, Rt=1.77 mins.

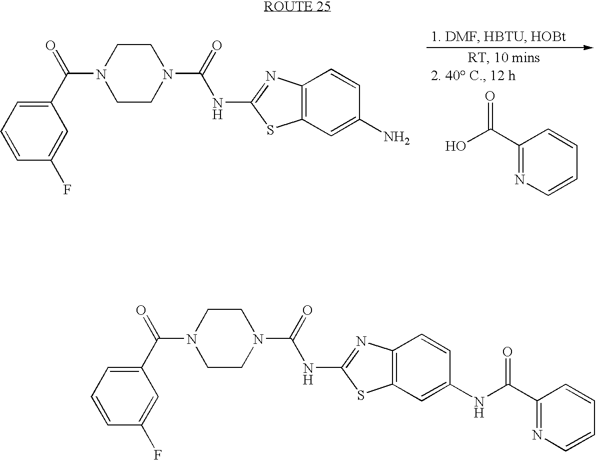

### Example 140

### 4-(3-Fluoro-benzoyl)-piperazine-1-carboxylic acid {6-[(pyridine-2-carbonyl)-amino]-benzothiazol-2-yl}-amide (Compound 140). Potency Range B

### General Procedure DD

To a stirred solution of pyridine-2-carboxylic acid (1 eqv, 3 mg, 0.025 mmol), HBTU (1.1 eqv, 10 mg, 0.028 mmol) and HOBt (1.1 eqv, 7 mg, 0.028 mmol) in DMF (2 ml) was added 4-(3-fluoro-benzoyl)-piperazine-1-carboxylic acid (6-amino-benzothiazol-2-yl)-amide (1.1 eqv, 11 mg, 0.028 mmol) after 10 mins at room temperature. The reaction mixture was stirred at 40° C. for 12 hrs and then concentrated at reduced pressure. The crude residue was purified by preparative HPLC to give product (1.3 mg, 9%, 0.003 mmol) as an off-white solid. LC/MS: 100% MH⁺, m/z 505, Rt=2.05 mins.

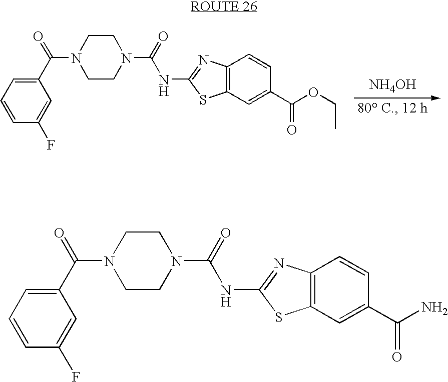

The following compound was made as illustrated in Route 26, as described in Route 17 Example 104 General procedure Y

### Example 141

### 2-{[4-(3-Fluoro-benzoyl)-piperazine-1-carbonyl]-amino}-benzothiazole-6-carboxylic acid amide (Compound 141). Potency Range B

In a similar fashion (Route 17: 104Y, 0.04 mmol of 2-{[4-(3-fluoro-benzoyl)-piperazine-1-carbonyl]-amino}-benzothiazole-6-carboxylic acid ethyl ester), gave the product (5 mg, 25%, 0.01 mmol) as a white solid. LC/MS: 100% MH⁺, m/z 428, Rt=1.69 mins.

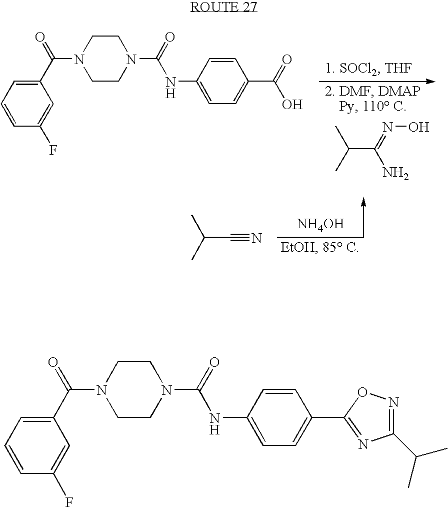

### Example 142

### 4-(3-Fluoro-benzoyl)-piperazine-1-carboxylic acid [4-(3-isopropyl-[1,2,4]oxadiazol-5-yl)-phenyl]-amide (Compound 142). Potency Range C

### General Procedure EE

To a solution of 4-{[4-(3-fluoro-benzoyl)-piperazine-1-carbonyl]-amino}-benzoic acid (1 eqv, 55 mg, 0.15 mmol) in THF (1 ml) was added thionyl chloride (5 eqv, 60 μl, 0.76 mmol) and the reaction mixture stirred for 3 hrs. Meanwhile, a pressure tube was charged with isobutyronitrile (1.1 eqv, 18 μl, 0.2 mmol), 50% aq hydroxylamine solution (5 eqv, 60 μl, 1.0 mmol) and EtOH (1 ml), sealed and heated to 85° C. for 7 hours. The solvent was evaporated and the resulting amidoxime combined with the preformed acid chloride intermediate, pyridine (4 eqv, 50 μl, 0.6 mmol), DMAP (0.1 eqv, 2.5 mg, 0.02 mmol) and DMF (2 ml). The reaction mixture was heated for 3 hours at 110° C., cooled to RT and concentrated at reduced pressure. Preparative HPLC purification gave the product (2 mg, 3%, 0.01 mmol) as a colourless oil. LC/MS: 100% MH⁺, m/z 438, Rt=2.00 mins.

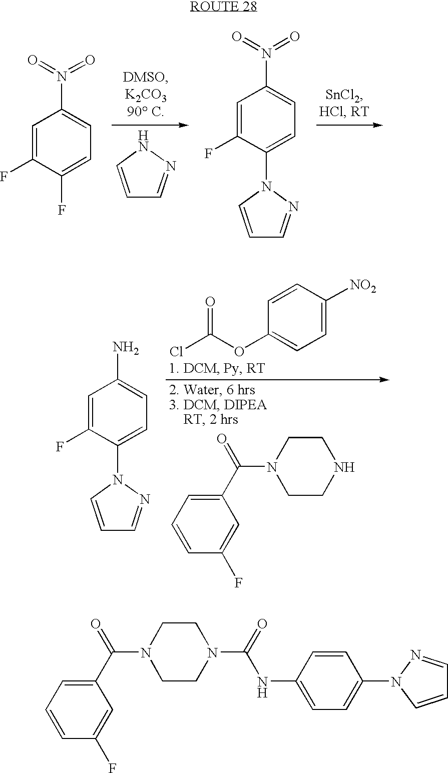

### Example 143

### 4-(3-Fluoro-benzoyl)-piperazine-1-carboxylic acid (4-pyrazol-1-yl-phenyl)-amide (Compound 143). Potency Range C

### General Procedure FF. 1-(2-Fluoro-4-nitro-phenyl)-1H-pyrazole

A pressure tube was charged with 1,2-difluoro-4-nitro-benzene (1 eqv, 0.5 g, 3.14 mmol), K₂CO₃ (2 eqv, 0.87 g, 6.29 mmol), pyrazole (1 eqv, 0.214 g, 3.14 mmol) and DMSO (10 ml), sealed and heated to 90° C. for 3 hours. The reaction mixture was cooled to room temperature and diluted with water (200 ml) resulting in precipitation of a white solid. The solid was collected by filtration, washed with water (20 ml) and dried under vacuum to give product (0.637 g, 97%, 3.06 mmol) as a yellow solid. LC/MS: 100% MH⁺, m/z 208, Rt=2.04 mins.

### General Procedure GG. 3-Fluoro-4-pyrazol-1-yl-phenylamine

To a solution of 1-(2-fluoro-4-nitro-phenyl)-1H-pyrazole (1 eqv, 0.05 g, 0.24 mmol) in concentrated HCl (2 ml) was added tin (II) chloride (5 eqv, 0.23 g, 1.21 mmol) and the resulting yellow solution stirred at room temperature. After 10 mins no yellow colouration remained. The reaction mixture was basified to pH 10 with 20% NaOH aq, resulting in precipitation of a white solid. The solid was collected by filtration, washed with water (5 ml) and dried under vacuum to give product (35 mg, 81%, 0.20 mmol) as a white solid. LC/MS: 100% MH⁺, m/z 178, Rt=1.55 mins.

The following compound was made as illustrated in Route 28, as described in Route 13 Example 76 General procedure U.

### 4-(3-Fluoro-benzoyl)-piperazine-1-carboxylic acid (4-pyrazol-1-yl-phenyl)-amide (Compound 143)

Following general procedure U (to scale 0.16 mmol of (3-fluoro-phenyl)-piperazin-1-yl-methanone) gave the title compound (2 mg, 3%, 0.004 mmol) as colourless oil. LC/MS: 100% MH⁺, m/z 412, Rt=1.96 mins.

The following compounds were made as described in Route 28 General procedures FF and GG and as described in Route 13 Example 76 General procedure U.

### Example 144

### 4-(3-Fluoro-benzoyl)-piperazine-1-carboxylic acid (4-imidazol-1-yl-phenyl)-amide (Compound 144). Potency Range C

### 1-(2-Fluoro-4-nitro-phenyl)-1H-imidazole

In a similar fashion (Route 28: 143FF, 3.143 mmol of 1,2-difluoro-4-nitro-benzene), gave the product (0.402 g, 61%, 1.941 mmol) as a yellow solid. LC/MS: 100% MH⁺, m/z 208, Rt=1.08 mins.

### 3-Fluoro-4-imidazol-1-yl-phenylamine

In a similar fashion (Route 28: 143GG, 0.24 mmol of 1-(2-fluoro-4-nitro-phenyl)-1H-imidazole), gave the product (43 mg, 100%, 0.24 mmol) as an off-white solid. LC/MS: 97% MH⁺, m/z 178, Rt=1.08 mins.

### 4-(3-Fluoro-benzoyl)-piperazine-1-carboxylic acid (4-imidazol-1-yl-phenyl)-amide (Compound 144)

In a similar fashion (Route 13: 76U, 0.24 mmol of (3-fluoro-phenyl)-piperazin-1-yl-methanone), gave the product (13 mg, 19%, 0.03 mmol) as a white solid after chromatography purification [SiO₂, EtOAc eluent]. LC/MS: 100% MH⁺, m/z 412, Rt=1.43 mins.

### Example 145

### 4-(3-Fluoro-benzoyl)-piperazine-1-carboxylic acid (4-[1,2,4]triazol-1-yl-phenyl)-amide (Compound 145). Potency Range C

### 1-(2-Fluoro-4-nitro-phenyl)-1H-[1,2,4]triazole

In a similar fashion (Route 28: 143FF, 3.143 mmol of 1,2-difluoro-4-nitro-benzene), gave the product (0.585 g, 89%, 2.81 mmol) as a yellow solid. LC/MS: 97% MH⁺, m/z 209, Rt=1.68 mins.

### 3-Fluoro-4-[1,2,4]triazol-1-yl-phenylamine

In a similar fashion (Route 28: 143GG, 0.24 mmol of 1-(2-fluoro-4-nitro-phenyl)-1H-[1,2,4]triazole), gave the product (33 mg, 76%, 0.18 mmol) as an off-white solid. LC/MS: 100% MH⁺, m/z 179, Rt=1.28 mins.

### 4-(3-Fluoro-benzoyl)-piperazine-1-carboxylic acid (4-[1,2,4]triazol-1-yl-phenyl)-amide (Compound 145)

In a similar fashion (Route 13: 76U, 0.24 mmol of (3-fluoro-phenyl)-piperazin-1-yl-methanone), gave the product (6 mg, 9%, 0.01 mmol) as a white solid after chromatography purification [SiO₂, EtOAc eluent]. LC/MS: 100% MH⁺, m/z 413, Rt=1.78 mins.

### Example 146

### 4-(3-Fluoro-benzoyl)-piperazine-1-carboxylic acid [3-fluoro-4-(4-pyridin-2-yl-piperazin-1-yl)-phenyl]-amide (Compound 146). Potency Range B

### 1-(2-Fluoro-4-nitro-phenyl)-4-pyridin-2-yl-piperazine

In a similar fashion (Route 28: 143FF, 1.833 mmol of 1,2-difluoro-4-nitro-benzene) but in the absence of base and solvent, gave the product (0.551 g, 99%, 1.83 mmol) as a brown solid. LC/MS: 100% MH⁺, m/z 303, Rt=1.53 mins.

### 3-Fluoro-4-(4-pyridin-2-yl-piperazin-1-yl)-phenylamineamine

In a similar fashion (Route 28: 143GG, 1.83 mmol of 1-(2-fluoro-4-nitro-phenyl)-4-pyridin-2-yl-piperazine), gave the product (381 mg, 76%, 1.39 mmol) as a pale orange solid. LC/MS: 100% MH⁺, m/z 273, Rt=0.92 mins.

### 4-(3-Fluoro-benzoyl)-piperazine-1-carboxylic acid [3-fluoro-4-(4-pyridin-2-yl-piperazin-1-yl)-phenyl]-amide (Compound 146)

In a similar fashion (Route 13: 76U, 0.24 mmol of (3-fluoro-phenyl)-piperazin-1-yl-methanone), gave the product (51 mg, 40%, 0.1 mmol) as a white solid after chromatography purification [SiO₂ 2 g isolute cartridge, EtOAc eluent]. LC/MS: 98% MH⁺, m/z 507, Rt=1.53 mins.

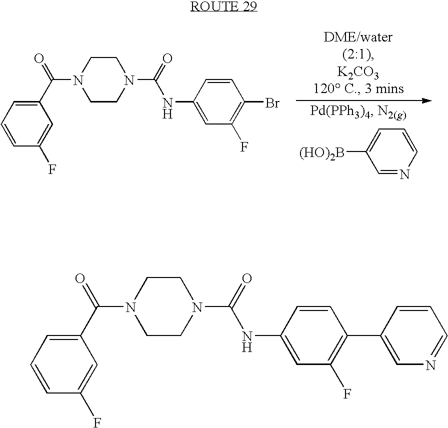

### Example 147

### 4-(3-Fluoro-benzoyl)-piperazine-1-carboxylic acid (3-fluoro-4-pyridin-3-yl-phenyl)-amide (Compound 147). Potency Range B

### General Procedure HH

A microwave reaction vessel was charged with 4-(3-fluoro-benzoyl)-piperazine-1-carboxylic acid (4-bromo-3-fluoro-phenyl)-amide (1 eqv, 15 mg, 0.035 mmol), Na₂CO₃ (1.5 eqv, 6 mg, 0.053 mmol), 3-pyridyl boronic acid (2 eqv, 7 mg, 0.07 mmol) and 2:1 DME/water (1 ml). The reaction mixture was degassed with nitrogen for 10 minutes, catalytic Pd(PPh₃)₄ (0.1 eqv, 3 mg, 0.004 mmol) added and the resulting mixture irradiated (300 W, 120° C.) for 3 mins. The reaction mixture was passed through a silica plug (eluting with EtOAc), the solvent evaporated and the crude residue purified by preparative HPLC to give product (2 mg, 12%, 0.01 mmol) as a colourless oil. LC/MS: 100% MH⁺, m/z 423, Rt=1.62 mins.

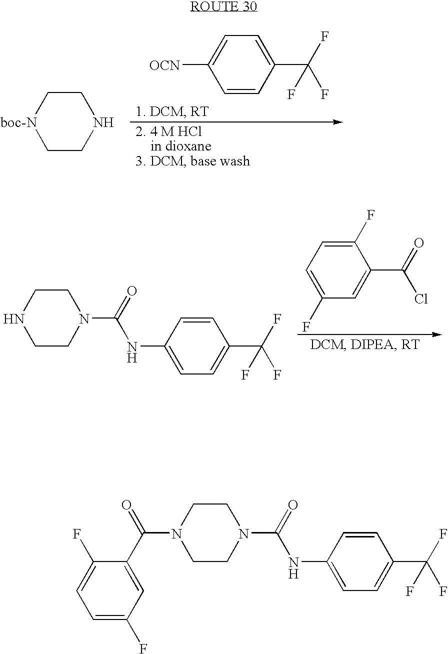

The following compounds were made as illustrated in Route 30, as described in Route 2 Example 15 General procedures F and E followed by Route 7 Example 56 General procedure M.

### Example 148

### Preparation of 4-(2,5-Difluoro-benzoyl)-piperazine-1-carboxylic acid (4-trifluoromethyl-phenyl)-amide (Compound 148). Potency Range C

### Piperazine-1-carboxylic acid (4-trifluoromethyl-phenyl)-amide

In a similar fashion (Route 2: 15F and E, 2.68 mmol of piperazine-1-carboxylic acid tert-butyl ester), gave the product (0.724 g, 98%, 2.66 mmol) as a colourless oil. LC/MS: 100% MH⁺, m/z 274, Rt=1.37 mins.

### 4-(2,5-Difluoro-benzoyl)-piperazine-1-carboxylic acid (4-trifluoromethyl-phenyl)-amide (Compound 148)

In a similar fashion (Route 7: 56M, 0.21 mmol of piperazine-1-carboxylic acid (4-trifluoromethyl-phenyl)-amide), gave the product (67 mg, 77%, 0.16 mmol) as a white solid. LC/MS: 98% MH⁺, m/z 414, Rt=2.19 mins.

### Example 149

### Preparation of 4-(3-Fluoro-benzoyl)-piperazine-1-carboxylic acid (4-trifluoromethyl-phenyl)-amide (Compound 149). Potency Range C

In a similar fashion (Route 7: 56M, 0.23 mmol of piperazine-1-carboxylic acid (4-trifluoromethyl-phenyl)-amide), gave the product (73 mg, 80%, 0.18 mmol) as a white solid. LC/MS: 95% MH⁺, m/z 396, Rt=2.16 mins.

The following compounds were made as illustrated in Route 30, as described in Route 7 Example 56 General procedure M using intermediate piperazine-1-carbothioic acid (6-morpholin-4-yl-pyridin-3-yl)-amide

### Example 150

### Preparation of 4-(2,5-Difluoro-benzoyl)-piperazine-1-carbothioic acid (6-morpholin-4-yl-pyridin-3-yl)-amide (Compound 150). Potency Range B

In a similar fashion (Route 7: 56M, 0.23 mmol of piperazine-1-carbothioic acid (6-morpholin-4-yl-pyridin-3-yl)-amide), gave the product (47 mg, 51%, 0.12 mmol) as an off-white solid. LC/MS: 100% MH⁺, m/z 408, Rt=1.12 mins.

### Example 151

### Preparation of 4-(3-Fluoro-benzoyl)-piperazine-1-carbothioic acid (6-morpholin-4-yl-pyridin-3-yl)-amide (Compound 151). Potency Range B

In a similar fashion (Route 7: 56M, 0.20 mmol of piperazine-1-carbothioic acid (6-morpholin-4-yl-pyridin-3-yl)-amide), gave the product (11 mg, 13%, 0.03 mmol) as an off-white solid. LC/MS: 89% MH⁺, m/z 430, Rt=1.10 mins.

### Example 152

### Preparation of 4-(2-Methyl-benzoyl)-piperazine-1-carbothioic acid (6-morpholin-4-yl-pyridin-3-yl)-amide (Compound 152). Potency Range B

In a similar fashion (Route 7: 56M, 0.19 mmol of piperazine-1-carbothioic acid (6-morpholin-4-yl-pyridin-3-yl)-amide), gave the product (33 mg, 40%, 0.08 mmol) as a pale yellow oil. LC/MS: 98% MH⁺, m/z 426, Rt=1.12 mins.

### Example 153

### Preparation of 4-(3-Nitro-benzoyl)-piperazine-1-carbothioic acid (6-morpholin-4-yl-pyridin-3-yl)-amide (Compound 153). Potency Range C

In a similar fashion (Route 7: 56M, 0.19 mmol of piperazine-1-carbothioic acid (6-morpholin-4-yl-pyridin-3-yl)-amide), gave the product (32 mg, 36%, 0.07 mmol) as a pale yellow oil. LC/MS: 94% MH⁺, m/z 457, Rt=1.11 mins.

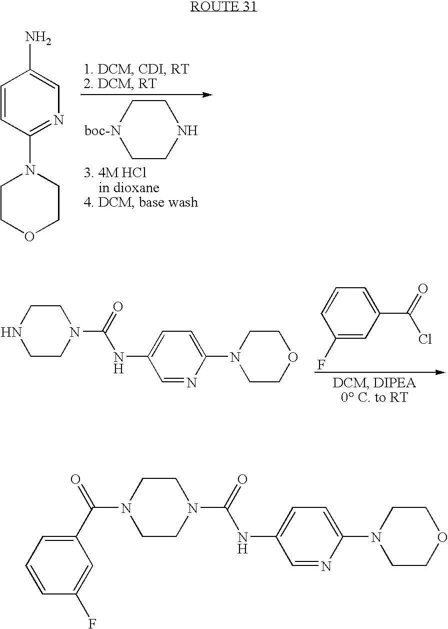

The following compounds were made as illustrated in Route 31, as described in Route 3 Example 38 General procedure G (using di-imidazol-1-yl-methanone), Route 5 Example 50 General procedure J and Route 7 Example 56 General procedure M.

### Example 154

### 4-(3-Fluoro-benzoyl)-piperazine-1-carboxylic acid (6-morpholin-4-yl-pyridin-3-yl)-amide (Compound 154). Potency Range B

### 4-(6-Morpholin-4-yl-pyridin-3-ylcarbamoyl)-piperazine-1-carboxylic acid tert-butyl ester

In a similar fashion (Route 3: 38G, using di-imidazol-1-yl-methanone (to scale 2.81 mmol of 6-morpholin-4-yl-pyridin-3-ylamine) gave the title compound (0.692 g, 63%, 1.77 mmol) as a pale pink solid. LC/MS: 100% MH⁺, m/z 392, Rt=1.03 mins.

### Piperazine-1-carboxylic acid (6-morpholin-4-yl-pyridin-3-yl)-amide

In a similar fashion (Route 5: 50J, 1.79 mmol of 4-(6-morpholin-4-yl-pyridin-3-ylcarbamoyl)-piperazine-1-carboxylic acid tert-butyl ester) gave the title compound (0.515 g, 99%, 1.77 mmol) as a purple solid. LC/MS: 100% MH⁺, m/z 292, Rt=0.63 mins.

### 4-(3-Fluoro-benzoyl)-piperazine-1-carboxylic acid (6-morpholin-4-yl-pyridin-3-yl)-amide (Compound 154)

In a similar fashion (Route 7: 56M, 0.21 mmol of piperazine-1-carboxylic acid (6-morpholin-4-yl-pyridin-3-yl)-amide), gave the product (47 mg, 55%, 0.11 mmol) as an off-white solid. LC/MS: 100% MH⁺, m/z 414, Rt=1.53 mins.

The following compounds were made as illustrated in Route 31, as described in Route 7 Example 56 General procedure M using intermediate piperazine-1-carboxylic acid (4-fluoro-phenyl)-amide.

### Example 155

### 4-(3-Fluoro-benzoyl)-piperazine-1-carboxylic acid (4-fluoro-phenyl)-amide (Compound 155). Potency Range C

In a similar fashion (Route 7: 56M, 0.27 mmol of piperazine-1-carboxylic acid (4-fluoro-phenyl)-amide), gave the product (55 mg, 59%, 0.16 mmol) as a white solid. LC/MS: 100% MH⁺, m/z 346, Rt=1.80 mins.

### Example 156

### 4-(2-Methyl-benzoyl)-piperazine-1-carboxylic acid (4-fluoro-phenyl)-amide (Compound 156). Potency Range C

In a similar fashion (Route 7: 56M, 0.27 mmol of piperazine-1-carboxylic acid (4-fluoro-phenyl)-amide), gave the product (69 mg, 75%, 0.20 mmol) as a white solid. LC/MS: 100% MH⁺, m/z 342, Rt=1.80 mins.

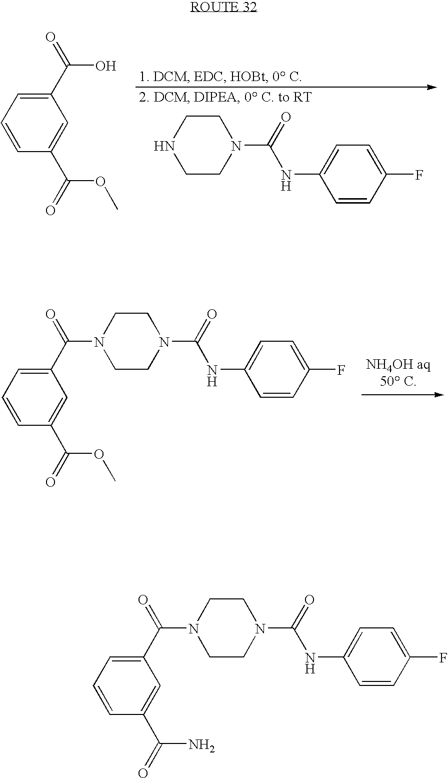

The following compound was made as illustrated in Route 32, as described in Route 2 Example 15 General procedure D using intermediate piperazine-1-carboxylic acid (4-fluoro-phenyl)-amide followed by Route 17 Example 104 General procedure Y.

### Example 157

### 4-(3-Carbamoyl-benzoyl)-piperazine-1-carboxylic acid (4-fluoro-phenyl)-amide (Compound 157). Potency Range C

### 3-[4-(4-Fluoro-phenylcarbamoyl)-piperazine-1-carbonyl]-benzoic acid methyl ester

In a similar fashion (Route 2: 15D, 1.64 mmol of piperazine-1-carboxylic acid (4-fluoro-phenyl)-amide), gave the product (0.580 g, 92%, 1.51 mmol) as a white solid. LC/MS: 100% MH⁺, m/z 386, Rt=1.18 mins.

### 4-(3-Carbamoyl-benzoyl)-piperazine-1-carboxylic acid (4-fluoro-phenyl)-amide (Compound 157)

In a similar fashion (Route 17: 104Y, 0.13 mmol of 3-[4-(4-fluoro-phenylcarbamoyl)-piperazine-1-carbonyl]-benzoic acid methyl ester), gave the product (9 mg, 19%, 0.02 mmol) as a white solid. LC/MS: 96% MH⁺, m/z 371, Rt=1.60 mins.

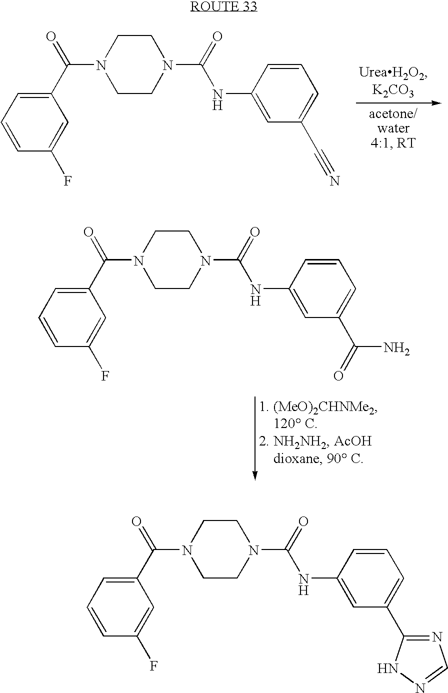

### Example 158

### 4-(3-Fluoro-benzoyl)-piperazine-1-carboxylic acid (3-carbamoyl-phenyl)-amide (Compound 158). Potency Range C

### General procedure II. 4-(3-Fluoro-benzoyl)-piperazine-1-carboxylic acid (3-carbamoyl-phenyl)-amide (Compound 158)

To a solution of 4-(3-fluoro-benzoyl)-piperazine-1-carboxylic acid (3-cyano-phenyl)-amide (1 eqv, 1.50 g, 4.26 mmol), K₂CO₃ (100 eqv, 58.8 g, 425.7 mmol) in acetone/water (4:1, 240 ml) was added urea hydrogen peroxide adduct (100 eqv, 40 g, 425.7 mmol) and the mixture stirred at room temperature for 20 hours. The solvent was evaporated and the resulting residue dissolved in DCM (500 ml), washed with water (3×100 ml) and evaporated to dryness to afford a white solid. The solid was washed with water (2×10 ml), cyclohexane (3×20 ml) and then dried at reduced pressure to give product (1.336 g, 85%, 3.61 mmol) as a white solid. LC/MS: 100% MH⁺, m/z 371, Rt=1.64 mins.

### Example 159

### Preparation of 4-(3-Fluoro-benzoyl)-piperazine-1-carboxylic acid [3-(2H-[1,2,4]triazol-3-yl)-phenyl]-amide (Compound 159). Potency Range C

General Procedure JJ.

A solution of 4-(3-fluoro-benzoyl)-piperazine-1-carboxylic acid (3-carbamoyl-phenyl)-amide (1 eqv, 0.5 g, 1.35 mmol) in N,N-dimethyl formamide dimethyl acetal (5 ml) was stirred at 120° C. for 2 hours. The solvent was evaporated and the residue re-dissolved in 1:1 dioxane/acetone (10 ml), and treated with hydrazine monohydrate (1.1 eqv, 74 mg, 1.48 mmol) at 90° C. for 3.5 hours. The solvent was evaporated at reduced pressure and the resulting residue purified by preparative HPLC to give product (9 mg, 2%, 0.023 mmol) as a yellow oil. LC/MS: 99% MH⁺, m/z 395, Rt=1.66 mins.

The following compounds were made as described in Route 33 General procedures II and JJ.

### Example 160

### 4-(3-Fluoro-benzoyl)-piperazine-1-carboxylic acid (3-carbamoyl-4-fluoro-phenyl)-amide (Compound 160). Potency Range C

In a similar fashion (Route 33: 158II, 4.26 mmol of 4-(3-fluoro-benzoyl)-piperazine-1-carboxylic acid (3-cyano-4-fluoro-phenyl)-amide), gave product (1.586 g, 96%, 4.08 mmol) as a white solid. LC/MS: 96% MH⁺, m/z 389, Rt=1.44 mins

### Example 161

### 4-(3-Fluoro-benzoyl)-piperazine-1-carboxylic acid [4-fluoro-3-(2H-[1,2,4]triazol-3-yl)-phenyl]-amide (Compound 161). Potency Range C

In a similar fashion (Route 33: 159JJ, 1.29 mmol of 4-(3-fluoro-benzoyl)-piperazine-1-carboxylic acid (3-carbamoyl-4-fluoro-phenyl)-amide), gave product (66 mg, 12%, 0.16 mmol) as a white solid. LC/MS: 99% MH⁺, m/z 413, Rt=1.66 mins

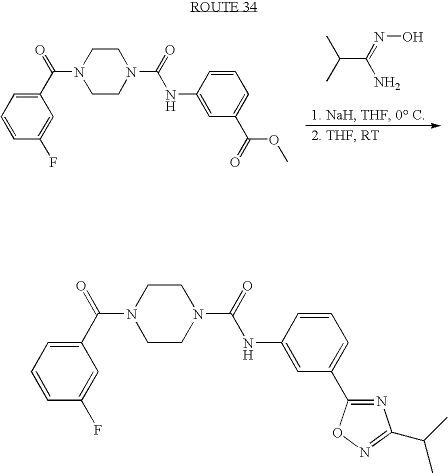

### Example 162

### 4-(3-Fluoro-benzoyl)-piperazine-1-carboxylic acid [3-(3-isopropyl-[1,2,4]oxadiazol-5-yl)-phenyl]-amide (Compound 162). Potency Range B

General Procedure KK.

To a solution of N-hydroxy-isobutyramidine (2 eqv, 106 mg, 1.04 mmol) in THF (1 ml) under nitrogen at 0° C. was added NaH in 60% mineral oil (2.5 eqv, 52 mg, 1.30 mmol) and the reaction mixture stirred for 1 hour. To this suspension was added dropwise 3-{[4-(3-fluoro-benzoyl)-piperazine-1-carbonyl]-amino}-benzoic acid methyl ester (1 eqv, 210 mg, 0.52 mmol) in THF and the reaction mixture then stirred at room temperature for 2 hours. The solvent was evaporated and the resulting residue dissolved in DCM (4 ml), washed with water (2×2 ml), dried (Na₂SO₄), filtered and evaporated at reduced pressure. Purification by preparative HPLC gave product (53 mg, 23%, 0.12 mmol) as a white solid. LC/MS: 100% MH⁺, m/z 438, Rt=1.98 mins.

### Example 163

### 4-(3-Fluoro-benzoyl)-piperazine-1-carboxylic acid [4-fluoro-3-(3-isopropyl-[1,2,4]oxadiazol-5-yl)-phenyl]-amide (Compound 163). Potency Range B

In a similar fashion (Route 34: 162KK, 0.52 mmol of 2-fluoro-5-{[4-(3-fluoro-benzoyl)-piperazine-1-carbonyl]-amino}-benzoic acid methyl ester), gave product from MeOH/water (1:2) recrystallisation (85 mg, 34%, 0.187 mmol) as a white solid. LC/MS: 96% MH⁺, m/z 456, Rt=2.18 mins

The following compounds were made in a similar way as described in Route 1 Example 1 General procedure C.

### Example 164

### Preparation of 4-Benzoyl-piperazine-1-carboxylic acid (4-trifluoromethyl-phenyl)-amide (Compound 164). Potency Range B

In a similar fashion (Route 1: 1 C, 1.0 mmol of phenyl-piperazin-1-yl-methanone), gave the product (142 mg, 38%, 0.38 mmol) as an off-white solid. LC/MS: 100% MH⁺, m/z 378, Rt=2.12 mins.

### Example 165

### Preparation of 4-(3-Chloro-benzoyl)-piperazine-1-carboxylic acid (4-trifluoromethyl-phenyl)-amide (Compound 165). Potency Range C

In a similar fashion (Route 1: 1 C, 1.0 mmol of (3-chloro-phenyl)-piperazin-1-yl-methanone), gave the product (124 mg, 30%, 0.30 mmol) as an off-white solid. LC/MS: 97% MH⁺, m/z 412, Rt=2.22 mins.

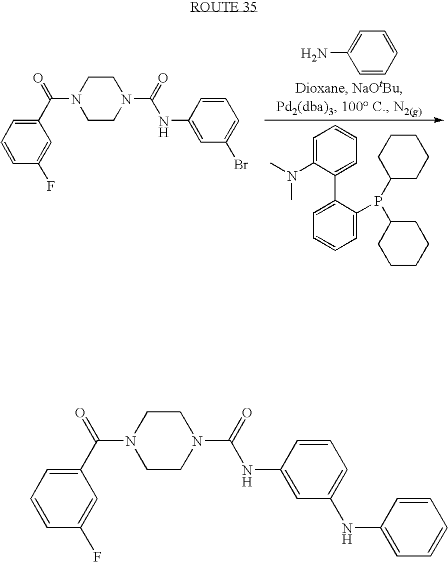

### Example 166

### 4-(3-Fluoro-benzoyl)-piperazine-1-carboxylic acid (3-phenylamino-phenyl)-amide (Compound 166). Potency Range C

General Procedure LL

A reaction vessel was charged with 4-(3-fluoro-benzoyl)-piperazine-1-carboxylic acid (3-bromo-phenyl)-amide (1 eqv, 30 mg, 0.074 mmol), NaO^{t}Bu (1.4 eqv, 10 mg, 0.103 mmol), phenylamine (1.2 eqv, 8.1 μl, 0.089 mmol), (2′-dicyclohexylphosphanyl-biphenyl-2-yl)-dimethyl-amine (0.12 eqv, 3.5 mg, 0.009 mmol), Pd₂(dba)₃ (0.04 eqv, 2.7 mg, 0.003 mmol) and dioxane (1 ml). The reaction mixture was degassed with nitrogen for 10 minutes and then heated to 100° C. for 12 hours. The reaction mixture was passed through a silica plug (eluting with EtOAc), the solvent evaporated and the crude residue purified by preparative HPLC to give product (0.5 mg, 1%, 0.001 mmol) as a colourless oil. LC/MS: 98% MH⁺, m/z 419, Rt=2.14 mins.

The following compound was made as described in Route 35 Example 166 General procedure LL.

### Example 167

### 4-(3-Fluoro-benzoyl)-piperazine-1-carboxylic acid [3-(4-fluoro-phenylamino)-phenyl]-amide (Compound 167). Potency Range C

In a similar fashion (Route 35: 166LL, 0.089 mmol of 4-fluoro-phenylamine), gave the product (3.1 mg, 10%, 0.009 mmol) as an off-white solid. LC/MS: 90% MH⁺, m/z 437, Rt=2.16 mins.

### Example 168

### Homogeneous, Biochemical Assay Monitoring the Activity of Human Prostaglandin D Synthase Based on the Glutathion-S-Transferase Activity of the Enzyme

Assay Principle:

Prostaglandin D2 synthesis by Prostaglandin D synthase (GST2) requires transfer of Glutathion (GSH) to the substrate, Prostaglandin H2. Indeed, Prostaglandin D Synthase belongs to the Glutathion-S-Transferase (GST) class of enzymes (Kanaoka et al., 1997, Cell 90, 1085-1095) and is capable of transfering GSH to different substrates. Since the Prostaglandin D2 synthesis is absolutely dependent on the GST activity of the enzyme, assays monitoring the GST activity should allow to identify specific inhibitors of Prostaglandin D2 synthetic activity. The assay principle used is based on enzymatic conjugation of GSH to Monochlorobimane (MCB), that becomes fluorescent upon this chemical modification. Therefore, the enzymatic reaction can be monitored by total fluorescence measurement.

Enzyme Purification:

As a source of enzyme, human Prostaglandin D Synthase was expressed as hexa-histidine fusion protein in E.coli and purified to >90% homogeneity by immobilized metal affinity chromatography.

In brief, the open reading frame of human Prostaglandin D Synthase was amplified from cDNA using the primer pair:

5′ CGCGGATCCCCAAACTACAAACTCACTTATTTTA 3′
and
5′ TCCCCCGGGCTAGAGTTTGGTTTGGGGCCTTC 3′

(utilized restriction sites underlined), and was cloned into the BamHI/SmaI sites of the vector pQE80L (Qiagen, Germany, cat. no. 32923). This construct allows prokaryotic expression of Prostaglandin D Synthase as fusion protein with a N-terminal hexa-histidine tag, referred to as NHis-Prostaglandin D Synthase.

Expression of NHis-Prostaglandin D Synthase was in E. coli TOP10F′ (Invitrogen, Germany, cat. no. C3030-03). Cells were grown in LB-Bouillon (Merck, Germany, cat. no. 1.10285) supplemented with 100 μg/ml ampicillin (Sigma, Germany, cat. no. A9518) at 37° C. When the culture had reached a density corresponding to an A₆₀₀ of 0.6, an equal volume of room temperature LB/ampicillin was added. Induction of NHis-Prostaglandin D Synthase expression was at 30° C. for 15 h with 1 mM isopropylthiogalactoside (IPTG, Roth, Germany, cat. no. 2316.4). Cells harvested by centrifugation were resuspended in 50 mM tris(hydroxymethyl)aminomethane hydrochloride pH 7.5 (Tris/HCl, Sigma, cat. no. T5941). Lysates were prepared by disruption of cells with a sonifier and subsequent clearing by centrifugation at 38000 g for 45 min at 4° C.

The lysate was applied to a column containing 25 ml Ni-NTA Superflow matrix (Qiagen, Germany, cat. no. 1018611) equilibrated with lysis buffer. Removal of unbound material was with 100 ml lysis buffer. Elution was with 50 ml of 50 mM Tris/HCl pH 7.5, 300 mM imidazol (Sigma, Germany, cat. no. I2399)/HCl pH 7.5). Peak fractions were pooled and the protein transferred into 50 mM Tris/HCl pH 7.5 by gel filtration on a HiPrep 26/10 desalting column (Amersham, Sweden, cat. no. 17-5087-01). Aliquots were shock frozen in liquid nitrogen and stored at −80° C.

Assay Protocol:

All assay components were diluted in reaction buffer, 50 mM MES/KOH (pH 6.1), 0.7 mM GSH (Sigma, G4251), 2 mM MgCl₂ (Sigma, M2670), 0.05% Pluronic F127 (Sigma, P2443), and stored on ice prior to use. Compounds were diluted in 90% DMSO yielding final assay concentrations from the picomolar to three digit micromolar range.

To start the enzymatic reaction, 35 μl of 51.8 nM Prostaglandin D Synthase was added to 35 μl of 1.036 mM MCB (Calbiochem, cat. no. 475906) and 2.5 μl of the respective compound dilution. The enzymatic reaction was carried out at room temperature in 384 well plates (PerkinElmer, cat. no. 6007279) for 60 minutes in the dark.

Total fluorescent measurement was carried out with excitation and emission wavelength of 390 and 460 nm, respectively (BMG Fluostar Optima).

Results

The GST2 inhibitors of general formula (I) typically have inhibitory potencies (IC₅₀'s) between 1 and 1,000 nM.

Compounds exhibiting IC₅₀ values in the range 1-10 nM are defined herein as: Potency Range A.

Compounds exhibiting IC₅₀ values in the range 11-100 nM are defined herein as: Potency Range B.

Compounds exhibiting IC₅₀ values in the range 101-1000 nM are defined herein as: Potency Range C.

The potency range for each of Compounds 1 to 167 is given beside the compound name in Examples 1 to 167 above.

### COMPARATIVE EXAMPLES

### Comparative Example Set 1

WO 99/07672 describes a series of compounds which are said to be modulators of K_{ATP} channels. In order to investigate this claim, we prepared a series of compounds which are exemplified in or are similar to the compounds of WO 99/07672.

### Comparative Example I

### Preparation of 4-(Furan-2-carbonyl)-piperazine-1-carboxylic acid (3,5-bis-trifluoromethyl-phenyl)-amide (Compound I)

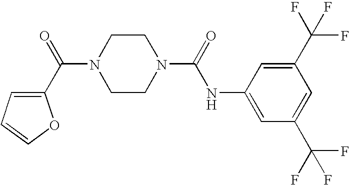

In a similar fashion (Route 1; 1C, 0.82 mmol furan-2-yl-piperazin-1-yl-methanone), gave the product (297 mg, 83%, 0.83 mmol) as a white solid. LC/MS: 100% MH⁺, m/z 436, Rt=2.28 mins.

### Comparative Example II

### Preparation of 4-(Furan-2-carbonyl)-piperazine-1-carboxylic acid (3,5-dichloro-phenyl)-amide (Compound II)

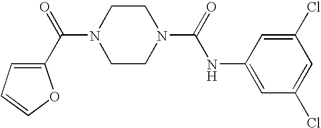

In a similar fashion (Route 1; 1C, 0.82 mmol furan-2-yl-piperazin-1-yl-methanone), gave the product (302 mg, 99%, 0.82 mmol) as a white solid. LC/MS: 100% MH⁺, m/z 368, Rt=2.14 mins.

### Comparative Example III

### Preparation of 4-(Furan-2-carbonyl)-piperazine-1-carboxylic acid (2-chloro-5-trifluoromethyl-phenyl)-amide (Compound III)

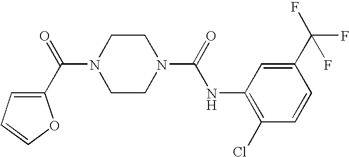

In a similar fashion (Route 1; 1C, 0.82 mmol furan-2-yl-piperazin-1-yl-methanone), gave the product (205 mg, 68%, 0.56 mmol) as a white solid. LC/MS: 100% MH⁺, m/z 402, Rt=2.13 mins.

### Comparative Example IV

### Preparation of 4-(Furan-2-carbonyl)-piperazine-1-carbothioic acid (3,5-dichloro-phenyl)-amide (Compound IV)

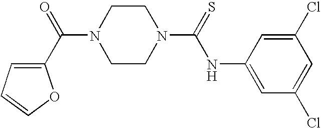

In a similar fashion (Route 1; 1C, 0.82 mmol furan-2-yl-piperazin-1-yl-methanone), gave the product (285 mg, 91%, 0.75 mmol) as an off white solid. LC/MS: 100% MH⁺, m/z 384, Rt=2.21 mins.

### Comparative Example V

### Preparation of 4-(Furan-2-carbonyl)-piperazine-1-carbothioic acid (2-chloro-5-trifluoromethyl-phenyl)-amide (Compound V)

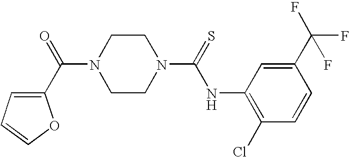

In a similar fashion (Route 1; 1C, 0.67 mmol furan-2-yl-piperazin-1-yl-methanone), gave the product (224 mg, 80%, 0.54 mmol) as an off white solid. LC/MS: 100% MH⁺, m/z 418, Rt=2.16 mins.

### Comparative Example VI

### Preparation of 4-(Furan-2-carbonyl)-piperazine-1-carbothioic acid (3,5-bis-trifluoromethyl-phenyl)-amide (Compound VI)

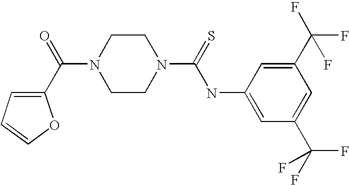

In a similar fashion (Route 1; 1C, 0.67 mmol furan-2-yl-piperazin-1-yl-methanone), gave the product (224 mg, 80%, 0.54 mmol) as an off white solid. LC/MS: 100% MH⁺, m/z 418, Rt=2.16 mins.

### Comparative Example VII

### Preparation of 4-Benzoyl-piperazine-1-carboxylic acid (3,5-dichloro-phenyl)-amide (Compound VII)

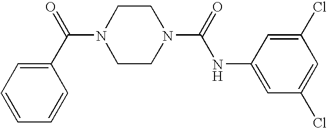

In a similar fashion (Route 1; 1C, 0.55 mmol phenyl-piperazin-1-yl-methanone), gave the product (207 mg, 100%, 0.55 mmol) as an off white solid. LC/MS: 100% MH⁺, m/z 378, Rt=2.23 mins.

### Comparative Example VIII

### Preparation of 4-Benzoyl-piperazine-1-carboxylic acid (2-chloro-5-trifluoromethyl-phenyl)-amide (Compound VIII)

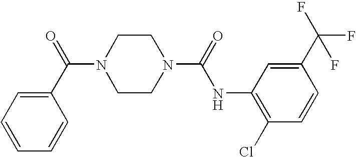

In a similar fashion (Route 1; 1C, 0.41 mmol phenyl-piperazin-1-yl-methanone), gave the product (115 mg, 68%, 0.28 mmol) as an off white solid. LC/MS: 100% MH⁺, m/z 412, Rt=2.21 mins.

### Comparative Example IX

### Preparation of 4-Benzoyl-piperazine-1-carbothioic acid (3,5-bis-trifluoromethyl-phenyl)-amide (Compound IX)

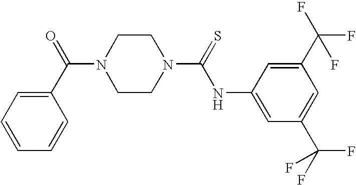

In a similar fashion (Route 1; 1C, 0.85 mmol phenyl-piperazin-1-yl-methanone), gave the product (374 mg, 95%, 0.81 mmol) as an off white solid. LC/MS: 100% MH⁺, m/z 462, Rt=2.42 mins.

### Comparative Example X

### Preparation of 4-Benzoyl-piperazine-1-carbothioic acid (3,5-dichloro-phenyl)-amide (Compound X)

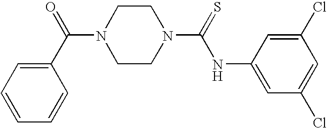

In a similar fashion (Route 1; 1C, 1.01 mmol phenyl-piperazin-1-yl-methanone), gave the product (393 mg, 99%, 1.00 mmol) as an off white solid. LC/MS: 100% MH⁺, m/z 394, Rt=2.31 mins.

### Comparative Example XI

### Preparation of 4-Benzoyl-piperazine-1-carbothioic acid (2-chloro-5-trifluoromethyl-phenyl)-amide (Compound XI)

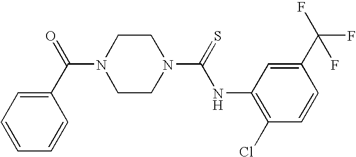

In a similar fashion (Route 1; 1C, 1.00 mmol phenyl-piperazin-1-yl-methanone), gave the product (240 mg, 56%, 0.56 mmol) as an off white solid. LC/MS: 98% MH⁺, m/z 428, Rt=2.24 mins.

### Comparative Example XII

### Preparation of 4-Benzoyl-piperazine-1-carbothioic acid (4-trifluoromethyl-phenyl)-amide (Compound XII)

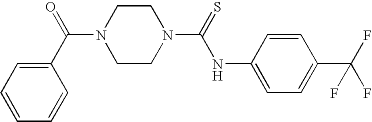

In a similar fashion (Route 1; 1C, 1.00 mmol phenyl-piperazin-1-yl-methanone), gave the product (311 mg, 79%, 0.79 mmol) as an off white solid. LC/MS: 100% MH⁺, m/z 394, Rt=2.23 mins.

### Example 169

### K_{ATP} Channel Mediated Binding Assay

The compounds of Comparative Examples I to XII and Compounds 59, 30, 22, 76 and 69 were tested for their effects in an in vitro receptor binding K_{ATP} channel assay.

Assay Principle:

The specific ligand binding to the receptor is defined as the difference between the total binding and the non-specific binding determined in the presence of an excess of unlabelled ligand. The results are expressed as a percent inhibition of control specific binding obtained in the presence of the test compounds.

General Procedures:

| | | Reference | |
|---|---|---|---|
| Assay | Origin | Compound | Bibliography |
| K⁺_{ATP} | rat cerebral | glibenclamide | Angel et al. Fundam. Clin. |
| channel | cortex | | Pharmacol., 1991, 5: 107-115. |

Experimental Conditions:

| | | | | Incuba- | Method of |
|---|---|---|---|---|---|
| Assay | Ligand | Conc. | Non Specific | tion | Detection |
| K⁺ ATP | [³H]gli- | 0.1 nM | glibenclamide | 60 min./ | Scintillation |
| channel | benclamide | | (1 μM) | 22° C. | counting |

In each experiment, the respective reference compound was tested concurrently with the test compounds in order to assess the assay suitability. It was tested at several concentrations (for IC₅₀ value determination), and the data were compared with historical values.

Assay Results:

Table 1 shows the K_{ATP} channel assay results for Compounds I to XII, which are all Comparative Examples covered by WO 99/07672 and for Compounds 59, 30, 22, 76 and 69, which are compounds of the present invention similar in structure to the compounds of WO 99/07672.

TABLE 1

| Compound | GST2 IC₅₀ | K⁺_{ATP} Channel (% Inhibition |
|---|---|---|
| Number | Range | of Control Specific Binding) |
| I | C | −1 |
| II | C | −17 |
| III | C | −8 |
| IV | C | −7 |
| V | C | −20 |
| VI | E | 4 |
| VII | B | −10 |
| VIII | C | −3 |
| IX | C | 10 |
| X | C | −10 |
| XI | B | −12 |
| XII | B | −14 |
| 59 | B | 15 |
| 30 | B | 16 |
| 22 | C | 1 |
| 76 | B | 20 |
| 69 | B | −22 |

In Table 1, results showing inhibition lower than 20% are not considered to be significant and are mostly attributable to variability of the signal around the control level. Results showing inhibition between 20% and 50% are indicative of weak to moderate effects. Low to moderate negative values have no real meaning and are attributable to variability of the signal around the control level. High negative values (≦−50%) are generally attributable to non-specific effects of the test compounds in the assay.

The K_{ATP} channel assay results set out in Table 1 show that at 10 μM compound concentration, no significant inhibition is observed for any of the compounds. This indicates that the Compounds tested are not K_{ATP} channel modulators. As such, according to the teaching of WO 99/07672, none of these compounds would be expected to be effective in the treatment of conditions such as diabetes, obesity and asthma.

It therefore appears that the compounds taught in WO 99/07672 do not have the pharmacological acitivity claimed in that document.

### Comparative Example Set 2

WO 02/059098 relates to a set of compounds which are said to be of use in the treatment of conditions such as diabetes, hyperlipidemia and obesity. We therefore prepared a set of similar compounds in order to investigate the optimal substitution pattern on the ring A of general formula (I)

The following compounds were made as described in Route 2 Example 15 General procedure D, using commercially available carboxylic acids.

### Comparative Example XIII

### Preparation of 2-[4-(4-Fluoro-phenylcarbamoyl)-piperazine-1-carbonyl]-benzoic acid methyl ester (Compound XVII)

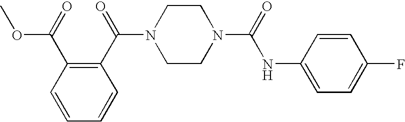

In a similar fashion (Route 2: 15D, 0.90 mmol of piperazine-1-carboxylic acid (4-fluoro-phenyl)-amide), gave the product (0.275 g, 80%, 0.72 mmol) as a white solid. LC/MS: 100% MH⁺, m/z 386, Rt=1.83 mins.

### Comparative Example XIV

### Preparation of 4-(2-Phenoxy-benzoyl)-piperazine-1-carboxylic acid (4-fluoro-phenyl)-amide (Compound XVIII)

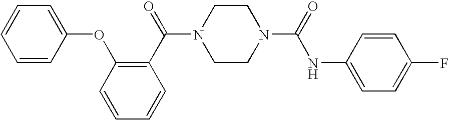

In a similar fashion (Route 2: 15D, 0.224 mmol of piperazine-1-carboxylic acid (4-fluoro-phenyl)-amide), gave the product (88 mg, 85%, 0.21 mmol) as a white solid. LC/MS: 100% MH⁺, m/z 420, Rt=2.13 mins.

The following compound was described in the preparation of Example 157 as illustrated in Route 32, as described in Route 2 Example 15 General procedure D.

### Comparative Example XV

### Preparation of 3-[4-(4-Fluoro-phenylcarbamoyl)-piperazine-1-carbonyl]-benzoic acid methyl ester (Compound XIX)

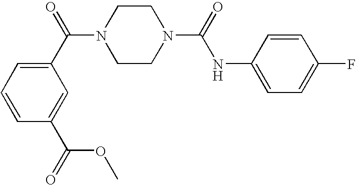

In a similar fashion (Route 2: 15D, 1.64 mmol of piperazine-1-carboxylic acid (4-fluoro-phenyl)-amide), gave the product (0.580 g, 92%, 1.51 mmol) as a white solid. LC/MS: 100% MH⁺, m/z 386, Rt=1.18 mins.

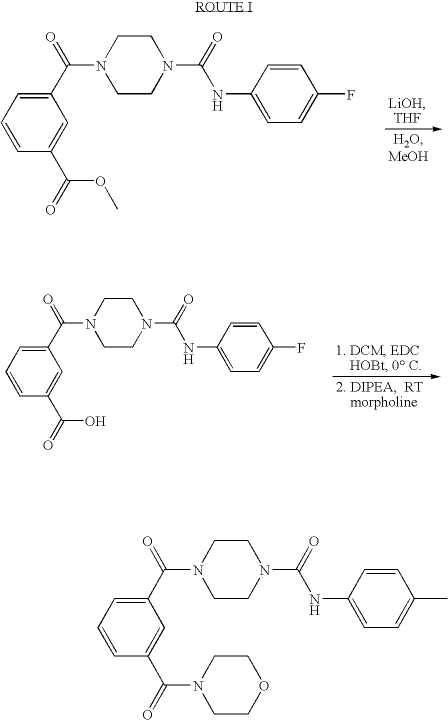

The following compound was made as illustrated in Route I, as described in Route 24 Example 138 General procedure CC and Route 16 Example 96 General procedure W

### Comparative Example XVI

### Preparation of 4-[3-(Morpholine-4-carbonyl)-benzoyl]-piperazine-1-carboxylic acid (4-fluoro-phenyl)-amide (Compound XVI)

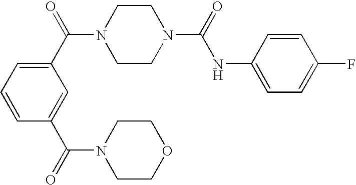

### 3-[4-(4-Fluoro-phenylcarbamoyl)-piperazine-1-carbonyl]-benzoic acid

In a similar fashion (Route 24: 138CC, 1.0 mmol of 3-[4-(4-fluoro-phenylcarbamoyl)-piperazine-1-carbonyl]-benzoic acid methyl ester), gave the product (0.237 g, 63%, 0.64 mmol) as a white solid. LC/MS: 97% MH⁺, m/z 372, Rt=1.66 mins.

### 4-[3-(Morpholine-4-carbonyl)-benzoyl]-piperazine-1-carboxylic acid (4-fluoro-phenyl)-amide (Compound XVI)

In a similar fashion (Route 16: 97X, 0.135 mmol of 3-[4-(4-fluoro-phenylcarbamoyl)-piperazine-1-carbonyl]-benzoic acid), gave the product (28 mg, 43%, 0.06 mmol) as a colourless oil. LC/MS: 100% MH⁺, m/z 441, Rt=1.65 mins.

The following compounds were made as illustrated in Route I, as described in Route 16 Example 97 General procedure X

### Comparative Example XVII

### Preparation of 4-(3-Diethylcarbamoyl-benzoyl)-piperazine-1-carboxylic acid (4-fluoro-phenyl)-amide (Compound XVII)

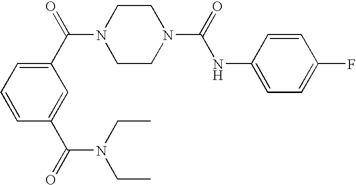

In a similar fashion (Route 16: 97X, 0.135 mmol of 3-[4-(4-fluoro-phenylcarbamoyl)-piperazine-1-carbonyl]-benzoic acid), gave the product (41 mg, 62%, 0.09 mmol) as a colourless oil. LC/MS: 98% MH⁺, m/z 427, Rt=1.75 mins.

### Comparative Example XVIII

### Preparation of 4-[3-(6-Fluoro-pyridin-3-ylcarbamoyl)-benzoyl]-piperazine-1-carboxylic acid (4-fluoro-phenyl)-amide (Compound XVIII)

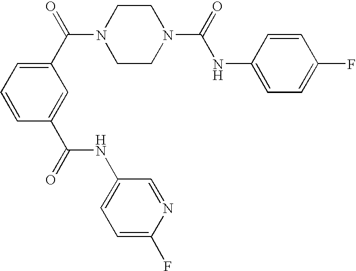

In a similar fashion (Route 16: 97X, 0.135 mmol of 3-[4-(4-fluoro-phenylcarbamoyl)-piperazine-1-carbonyl]-benzoic acid), gave the product (29 mg, 42%, 0.06 mmol) as a white solid. LC/MS: 100% MH⁺, m/z 465, Rt=1.79 mins.

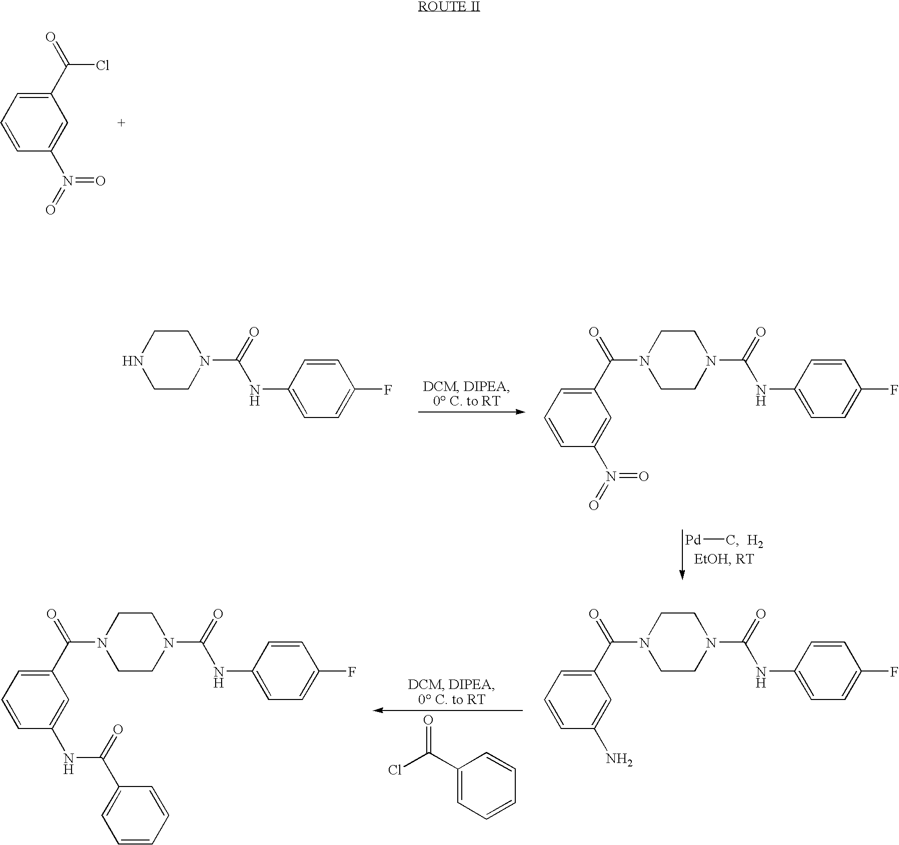

The following compounds were made as illustrated in Route II, but described in Route 7 Example 56 General procedure M, Route 10 Example 61 General procedure Q and Route 7 Example 56 General procedure M.

### Comparative Example XIX

### 4-(3-Benzoylamino-benzoyl)-piperazine-1-carboxylic acid (4-fluoro-phenyl)-amide (Compound XIX)

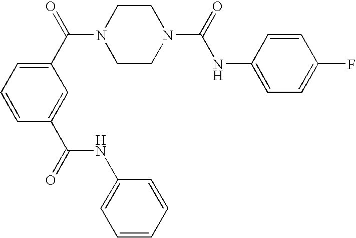

### 4-(3-Nitro-benzoyl)-piperazine-1-carboxylic acid (4-fluoro-phenyl)-amide

In a similar fashion (Route 7: 56M, 0.269 mmol of piperazine-1-carboxylic acid (4-fluoro-phenyl)-amide), gave the product (92 mg, 92%, 0.247 mmol) as a yellow solid. LC/MS: 100% MH⁺, m/z 373, Rt=1.80 mins.

### 4-(3-Amino-benzoyl)-piperazine-1-carboxylic acid (4-fluoro-phenyl)-amide

In a similar fashion (Route 10: 61Q, 0.247 mmol of 4-(3-nitro-benzoyl)-piperazine-1-carboxylic acid (4-fluoro-phenyl)-amide), gave the product (69 mg, 81%, 0.20 mmol) as a colourless oil. LC/MS: 95% MH⁺, m/z 343, Rt=1.51 mins.

### 4-(3-Benzoylamino-benzoyl)-piperazine-1-carboxylic acid (4-fluoro-phenyl)-amide (Compound XIX)

In a similar fashion (Route 7: 56M, 0.06 mmol of 4-(3-amino-benzoyl)-piperazine-1-carboxylic acid (4-fluoro-phenyl)-amide), gave the product (12 mg, 45%, 0.03 mmol) as an off-white solid. LC/MS: 96% MH⁺, m/z 447, Rt=1.86 mins.

The following compound wase made as described in Route 20 Example 125 General procedure Z.

### Comparative Example XX

### 4-(3-Benzenesulfonylamino-benzoyl)-piperazine-1-carboxylic acid (4-fluoro-phenyl)-amide (Compound XX)

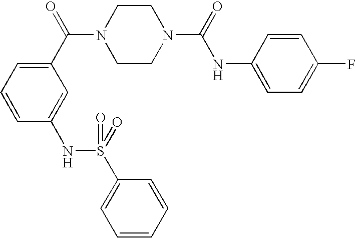

In a similar fashion (Route 20: 125Z, 0.06 mmol of 4-(3-amino-benzoyl)-piperazine-1-carboxylic acid (4-fluoro-phenyl)-amide), gave the product (14 mg, 50%, 0.03 mmol) as an off-white solid. LC/MS: 100% MH⁺, m/z 483, Rt=1.88 mins.

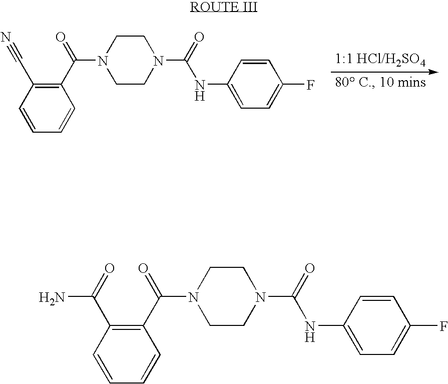

### Comparative Example XXI

### 4-(2-Carbamoyl-benzoyl)-piperazine-1-carboxylic acid (4-fluoro-phenyl)-amide (Compound XXI)

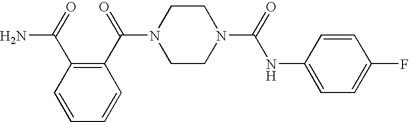

A cold (0° C.) 1:1 solution of concentrated HCl and concentrated H₂SO₄ (0.5 ml) was added dropwise to 4-(2-cyano-benzoyl)-piperazine-1-carboxylic acid (4-fluoro-phenyl)-amide (1 eqv, 20 mg, 0.06 mmol) and the resulting mixture heated to 80° C. for 10 mins then cooled to room temperature. The reaction mixture was extracted with EtOAc (2×5 ml) and the organic layer washed with 1M K₂CO₃ aq (5 ml), brine (5 ml), dried (MgSO₄), filtered and evaporated at reduced pressure. Flash column chromatography [SiO₂, eluting with 95:5 DCM/MeOH] gave product (4 mg, 19%, 0.01 mmol) of colourless oil. LC/MS: 96% MH⁺, m/z 371, Rt=1.61 mins.

The following compound was made as described in Route 24 Example 138 General procedure CC.

### Comparative Example XXII

### 2-[4-(4-Fluoro-phenylcarbamoyl)-piperazine-1-carbonyl]-benzoic acid (Compound XXII)

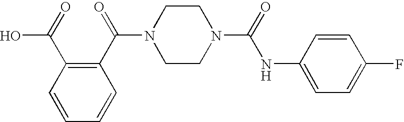

In a similar fashion (Route 24: 138CC, 0.11 mmol of 2-[4-(4-fluoro phenylcarbamoyl)-piperazine-1-carbonyl]-benzoic acid methyl ester), gave the product (35 mg, 85%, 0.09 mmol) as a white solid. LC/MS: 100% Mfe, m/z 372, Rt=1.69 mins.

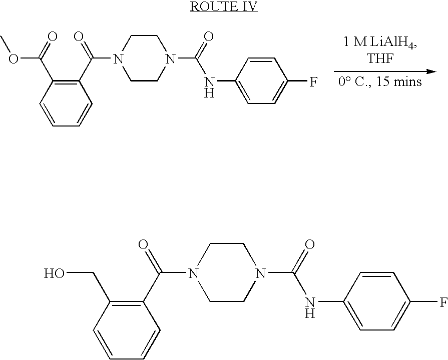

### Comparative Example XXIII

### 4-(2-Hydroxymethyl-benzoyl)-piperazine-1-carboxylic acid (4-fluoro-phenyl)-amide (Compound XXIII)

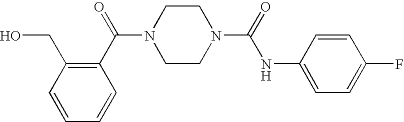

To a solution of 2-[4-(4-fluoro-phenylcarbamoyl)-piperazine-1-carbonyl]-benzoic acid methyl ester (1 eqv, 50 mg, 0.13 mmol) in dry THF (2 ml) at 0° C. was added a 1M solution of LiAlH₄ in THF (1.1 eqv, 143 μl, 0.143 mmol) and the reaction mixture stirred for 15 mins. The reaction mixture was quenched by the addition of water (2 drops) and 2M NaOH aq (2 drops) and the resulting inorganic precipitate removed by filtration. The evaporated filtrate was purified by flash column chromatography [SiO₂, eluting with EtOAc] to give product (20 mg, 43%, 0.06 mmol) of white solid. LC/MS: 100% MH⁺, m/z 358, Rt=1.67 mins.

### Comparative Example XXIV

The Assay of Example 168 was repeated for the Compounds of Comparative Examples XIII to XXIII

Assay Results:

The GST2 inhibitory potency (IC₅₀) assay results for Comparative Examples XIII to XXVII are shown in Table 2 and indicate that substitution of moiety “A” in general formula (I) with small to moderate sized groups results in a loss activity. Therefore much better activity is obtained when the size of the moiety “A” in general formula (I) is limited to less than that claimed as required for PPARγ activity.

TABLE 2

| Compound | GST2 inhibition |
|---|---|
| Number | IC₅₀ (μM) |
| XIX | 16.2 |
| XXIII | 16.25 |
| XXI | 16.785 |
| XVIII | 19.465 |
| XX | 19.585 |
| XIII | 28.225 |
| XIV | 180.45 |
| XV | 1000 |
| XVI | 1000 |
| XVII | 1000 |
| XXII | 1000 |

### Example 170

### Selected GST2 Inhibitors Screened Against PPARγ and K_{ATP} Channel Modulator Assays

Selected GST2 inhibitors of General Formula (I) were screened in a PPARγ assay to determine their modulator effects.

Assay Principle:

The specific ligand binding to the receptor is defined as the difference between the total binding and the non-specific binding determined in the presence of an excess of unlabelled ligand. The results are expressed as a percent inhibition of control specific binding obtained in the presence of the test compounds.

General Procedures:

| | | Reference | |
|---|---|---|---|
| Assay | Origin | Compound | Bibliography |
| PPARγ (h) | human | rosiglitazone | Ferry et al. Eur. J. |
| | recombinant | | Pharmacol., 2001, |
| | (E. coli) | | 417: 77-89 |

Experimental Conditions:

| | | | | Incuba- | Method of |
|---|---|---|---|---|---|
| Assay | Ligand | Conc. | Non Specific | tion | Detection |
| PPARγ | [³H]rosi- | 10 nM | rosiglitazone | 90 min./ | Scintillation |
| (h) | glitazone | | (10 μM) | 4° C. | counting |

In each experiment, the respective reference compound was tested concurrently with the test compounds in order to assess the assay suitability. It was tested at several concentrations (for IC₅₀ value determination), and the data were compared with historical values.

Assay Results:

The results shown in Table 3 below indicate that the selected GST2 inhibitors of General Formula (I) are not modulators of PPARγ.

TABLE 3

| | | PPARγ (% | |
|---|---|---|---|
| | GST2 | Inhibition of | K+ATP Channel (% |
| Compound | IC50 | Control Specific | Inhibition of Control |
| Number | range | Binding) | Specific Binding) |
| 59 | B | 15 | 12 |
| 30 | B | 16 | 13 |
| 22 | C | 1 | 14 |
| 76 | B | 20 | −2 |
| 69 | B | −22 | −13 |

### Example 171

### Pharmacological Study

Treatment of B6.V Lep^{ob} mice (commonly referred to ob/ob mice) with a small molecule GST2 inhibitor (Compound 22) to demonstrate that compounds of General Formula I are likely to be useful in the treatment of metabolic syndrome, especially obesity.

Study Design:

16 Male ob/ob mice were obtained from Charles River in Belgium at an approximate age of 6 weeks and with an approximate mean body weight of 35 g at the beginning of substance administration. Mice were housed 4 per cage at a temperature of 23° C.±1 and a day/night cycle of 14/10 hours (05:00-19:00-05:00), with ad libitum access to standard chow and tap water. Mice were allowed to adapt to these conditions for 8 days prior to the start of treatment. Animals were administered substance by oral gavage in the morning at 07:00, immediately after determining body weight and food consumption. In the evening substance was administered at 19:00. One group of 8 animals were administered vehicle, 0.3% methylcellulose in saline, and the second group of 8 animals were administered Compound 22 (120 mg/kg/day) suspended in the vehicle. Body weight and food consumption were recorded daily during 12 days of treatment and the results are presented in FIG. 1, which is a body weight curve graph. This illustrates a 7% reduced weight gain in Compound 22 administered ob/ob mice, despite unaltered food intake. The results show that Compound 22 is effective in decreasing the weight gain in ob/ob mice and supports claims herein that compounds of General Formula I are useful in the treatment of metabolic syndrome, particularly obesity.

### Example 171

### Selected GST2 Inhibitors from Formula I Assessed in Mouse Microsomal Metabolism Assay

Selected GST2 inhibitors of General Formula (I) were screened in a mouse hepatic microsome assay to determine microsomal metabolism.

Materials and Methods:

Test Articles were prepared in 50% acetonitrile.

Biological Material:

Mouse hepatic microsomes supplied by BD GenTest Inc or an equivalent reputable supplier.

Experimental Procedures:

Test Articles are added to incubation plates containing phosphate buffer pH 7.4 and microsomes at a suitable protein concentration. Incubation plates are placed on a shaking water bath at 37° C. for 5 minutes pre-incubation. An aliquot of the reaction mixture is taken and added to acetonitrile (3:1). This is designated time t=−1. NADPH regenerating system in phosphate buffer pH 7.4 is added to initiate reactions. Aliquots from the incubation wells are taken at a number of time points and added to acetonitrile. Control incubations contained no microsomes or compounds with known microsomal stabilities. Samples were subject to centrifugation to remove denatured protein and the supernatants were analysed by LCMS.

Data Analysis:

Half lives were calculated from the log vs time transformed data according to the following equation:

Half life (t_{1/2})=−(0.693/λ)

Where λ is the slope of the linear regression line under first order (Test Article concentration <Km) conditions.

The compounds XXIII to XXXI illustrated in Table 4 were made using routes disclosed herein from commercially available starting materials.

TABLE 4

| | | | Mouse |
|---|---|---|---|
| Compound | | | t½ |
| Number | Structure | Name | (mins) |
| 21 | 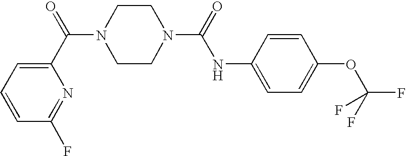 | 4-(6-Fluoro-pyridine-2-carbonyl)- piperazine-1-carboxylic acid (4- trifluoromethoxy-phenyl)-amide | >60 |
| 22 | 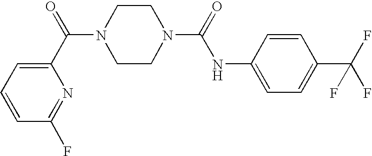 | 4-(6-Fluoro-pyridine-2-carbonyl)- piperazine-1-carboxylic acid (4- trifluoromethyl-phenyl)-amide | >60 |
| 39 | 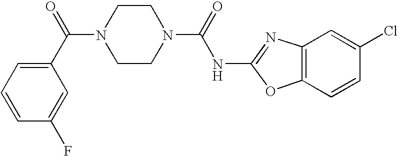 | 4-(3-Fluoro-benzoyl)-piperazine-1- carboxylic acid (5-chloro-benzooxazol- 2-yl)-amide | >60 |
| 50 | 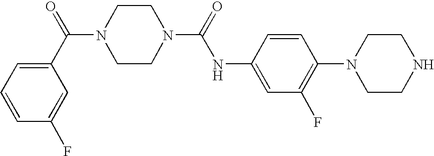 | 4-(3-Fluoro-benzoyl)-piperazine-1- carboxylic acid (3-fluoro-4-piperazin-1- yl-phenyl)-amide | >60 |
| 52 | 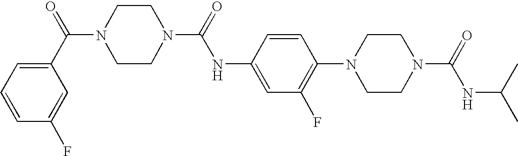 | 4-(3-fluorobenzoyl)-N-{3-fluoro-4-[4- (isopropylcarbamoyl) piperazin-1- yl]phenyl}piperazine-1-carboxamide | >60 |
| 63 | 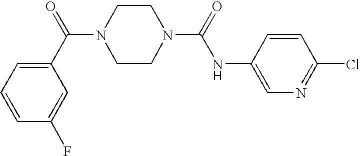 | 4-(3-Fluoro-benzoyl)-piperazine-1- carboxylic acid (6-chloro-pyridin-3- yl)-amide | >60 |
| 101 | 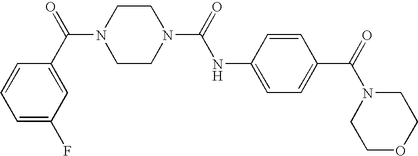 | 4-(3-Fluoro-benzoyl)-piperazine-1- carboxylic acid [4-(morpholine-4- carbonyl)-phenyl]-amide | >60 |
| 105 | 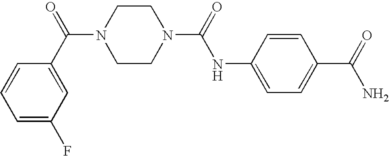 | 4-(3-Fluoro-benzoyl)-piperazine-1- carboxylic acid (4-carbamoyl-phenyl)- amide | >60 |
| 109 | 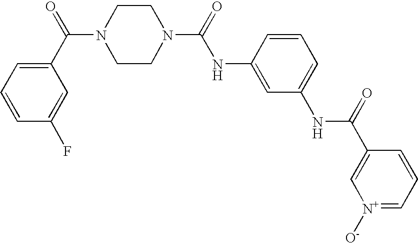 | 4-(3-Fluoro-benzoyl)-piperazine-1- carboxylic acid {3-[(1-oxy-pyridine- 3-carbonyl)-amino]-phenyl}-amide | >60 |
| 123 | 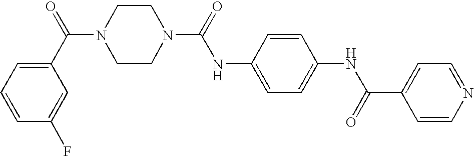 | 4-(3-Fluoro-benzoyl)-piperazine-1- carboxylic acid {4-[(pyridine-4- carbonyl)-amino]-phenyl}-amide | >60 |
| 126 | 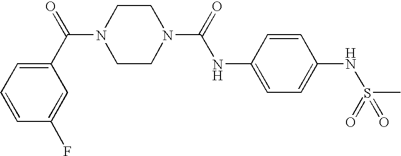 | 4-(3-Fluoro-benzoyl)-piperazine-1- carboxylic acid (4-methanesulfonyl- amino-phenyl)-amide | >60 |
| 154 | 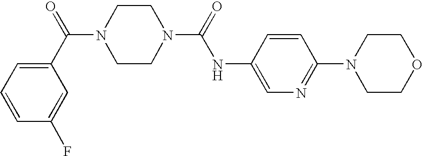 | 4-(3-Fluoro-benzoyl)-piperazine-1- carboxylic acid (6-morpholin-4-yl- pyridin-3-yl)-amide | >60 |
| XXIII | 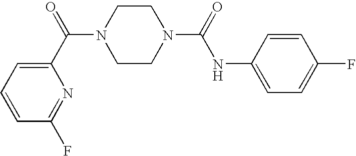 | 4-(6-Fluoro-pyridine-2-carbonyl)- piperazine-1-carboxylic acid (4- fluoro-phenyl)-amide | >60 |
| 155 | 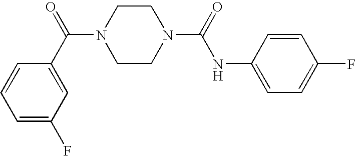 | 4-(3-Fluoro-benzoyl)-piperazine-1- carboxylic acid (4-fluoro-phenyl)-amide | 59.4 |
| 60 | 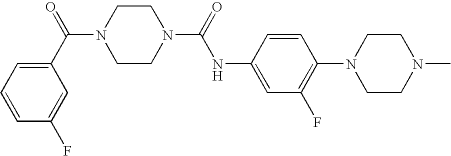 | 4-(3-Fluoro-benzoyl)-piperazine-1- carboxylic acid [3-fluoro-4-(4-methyl- piperazin-1-yl)-phenyl]-amide | 58.7 |
| 156 | 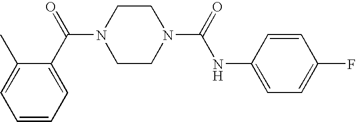 | 4-(2-Methyl-benzoyl)-piperazine-1- carboxylic acid (4-fluoro-phenyl)-amide | 31.4 |
| XXIV | 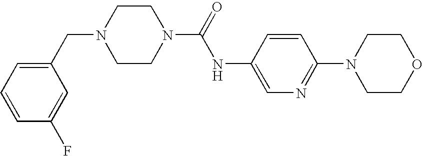 | 4-(3-Fluoro-benzyl)-piperazine-1- carboxylic acid (6-morpholin-4-yl- pyridin-3-yl)-amide | 31.4 |
| XXV | 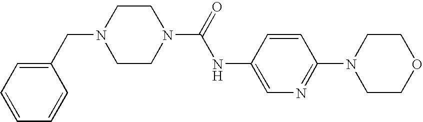 | 4-Benzyl-piperazine-1-carboxylic acid (6-morpholin-4-yl-pyridin-3-yl)-amide | 27.6 |
| XXVI | 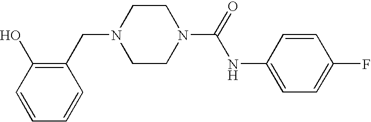 | 4-(2-Hydroxy-benzyl)-piperazine-1- carboxylic acid (4-fluoro-phenyl)-amide | 21.9 |
| XXVII | 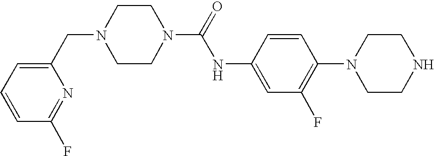 | 4-(6-Fluoro-pyridin-2-ylmethyl)- piperazine-1-carboxylic acid (3-fluoro- 4-piperazin-1-yl-phenyl)-amide | 20.9 |
| XXVIII | 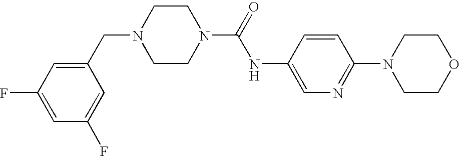 | 4-(3,5-Difluoro-benzyl)-piperazine-1- carboxylic acid (6-morpholin-4-yl- pyridin-3-yl)-amide | 15.5 |
| XXIX | 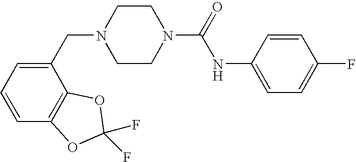 | 4-(2,2-Difluoro-benzo[1,3]dioxol-4- ylmethyl)-piperazine-1-carboxylic acid (4-fluoro-phenyl)-amide | 12.1 |
| XXX | 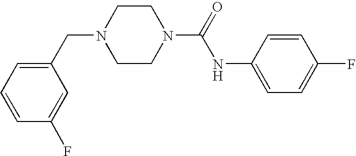 | 4-(3-Fluoro-benzyl)-piperazine-1- carboxylic acid (4-fluoro-phenyl)-amide | 10.6 |
| XXXI | 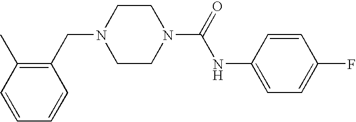 | 4-(2-Methyl-benzyl)-piperazine-1- carboxylic acid (4-fluoro-phenyl)-amide | 4.4 |

1. A compound of general formula (I): 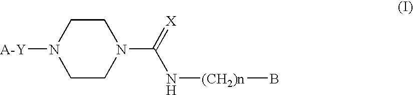
wherein
X is O or S;
Y is C=O or CR¹R²
R¹ and R² are each independently halogen or alternatively R¹ and R² may combine to form an alkylene chain —(CH₂)ₘ—, where m is 2 to 4;
A is a 5 to 10 membered aromatic or heteroaromatic ring system which may optionally be substituted with one or more substituents chosen from halogen, C₁₋₃ alkyl, C₁₋₃ haloalkyl, CN, OR³, R³, SR³, SOR³, SO₂R³, NO₂, CONH₂, CH₂OR³, CH₂NR³R⁴ or NR³R⁴;
R³ and R⁴ are each independently hydrogen, C₁₋₄ alkyl or C₁₋₄ haloalkyl;
or when there are two OR³ substituents on adjacent positions of the group A, the two R³ groups may combine to form an alkylene chain or alkenylene chain having from 1 to 3 carbon atoms and optionally substituted by one or more halogen atoms;
n is 0 or 1;
B is a 5 to 10 membered aromatic or heteroaromatic ring system optionally substituted with one or more substitutents chosen from halogen, CN, NO₂, R¹⁰, —OR¹⁰, —CO₂R¹⁰, —COR¹⁰, —CONR¹⁰R¹¹, —NR¹⁰COR¹¹, —NR¹⁰CO₂R¹¹, —SO₂NR¹⁰R¹¹, —SONR¹⁰R¹¹, —SOR¹⁰, —SO₂R¹⁰, —NR¹⁰SO₂NR¹¹R¹², —SR¹⁰, —NR¹⁰R¹¹, —OCOR¹⁰, —NR¹⁰SO₂R¹¹, —NR¹⁰SOR¹¹, —N(SO₂R¹⁰)₂, —NR¹⁰(CH₂)_{q}CO₂R¹¹, or —O—(CH₂)_{q}T;
R¹⁰, R¹¹ and R¹² are each independently H, or C₁₋₆ alkyl, C₁₋₆ haloalkyl, or a group T; wherein T is a C₃₋₇ cycloalkyl, C₃₋₇ heterocyclyl, —C₁₋₆ alkyl(C₃₋₇ cycloalkyl), —C₁₋₆ alkyl(C₃₋₇ heterocyclyl), C₅₋₁₀ aromatic or C₅₋₁₀ heteroaromatic group, any of which is optionally substituted with one or more substituents chosen from C₁₋₆ alkyl, C₁₋₆ haloalkyl, O—C₁₋₆ alkyl, O—C₁₋₆ haloalkyl, halogen, CN, NO₂, R³, —CO₂R³, —COR³, —CONR³R⁴, —NR³COR¹¹, —NR³CO₂R⁴, —SO₂NR³R³R⁴, —SONR³R⁴, —SOR³, —SO₂R³, —NR³SO₂NR⁴R⁵, —SR³, —NR³R⁴, —OCOR³, —NR³SO₂R⁴, —NR³SOR⁴, —N(SO₂R³)₂ or —NR³(CH₂)_{q}CO₂R⁴;
wherein R³ and R⁴ are as defined above and R⁵ is as defined for R³ and R⁴;
or when there are two OR¹⁰ substituents on adjacent positions of the group B, the two R¹⁰ groups may combine to form an alkylene chain or alkenylene chain having from 1 to 3 carbon atoms;
q is an integer of 1 to 6
or a pharmacologically acceptable salt, hydrate, solvate, complex prodrug thereof;
provided that:
B is not benzisoxazole;
when A is phenyl or pyridyl, B is not phenyl disubstituted with a methoxy and a piperazinyl group;
when A is furyl, B is not phenyl substituted with halogen or —CF₃;
when A is unsubstituted phenyl, B is not unsubstituted phenyl or phenyl substituted with trifluoromethyl.

2. A compound as claimed in claim 1, wherein A is naphthyl or a monocyclic aromatic or heteroaromatic ring system with 5 or 6 ring atoms.

3. A compound as claimed in claim 2, wherein A is a monocyclic aromatic or heteroaromatic groups with 6 ring atoms.

4. A compound as claimed in claim 3, wherein A is phenyl, pyridyl or pyrazinyl optionally substituted with one or more halogen, NO₂, CN, CONH₂, CH₂OH, CH₂NR³R⁴, NR³R⁴, SR³, SOR³, SO₂R³, C₁-C₃ alkyl, C₁-C₃ haloalkyl, O(C₁-C₃ alkyl) or O—(C₁-C₃ haloalkyl) groups, where R³ and R⁴ are hydrogen or methyl.

5. A compound as claimed in claim 1, wherein A is unsubstituted or substituted with one or two substituents chosen from chloro, fluoro, trifluoromethyl, methoxy, ethoxy, trifluoromethoxy, difluoromethoxy, cyano and nitro.

6. A compound as claimed in claim 1, wherein A is a 2- or 3-pyridyl group.

7. A compound as claimed in claim 1, wherein Y is C═O.

8. A compound as claimed in claim 1, wherein n is 0.

9. A compound of general formula (Ia): 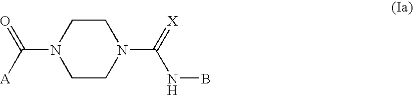
wherein A is phenyl, 2-pyridyl or 3-pyridyl optionally substituted with one or two substituents chosen from chloro, fluoro, trifluoromethyl, methoxy, ethoxy, trifluoromethoxy, difluoromethoxy, cyano and nitro; and B and X are as defined for general formula (I) in claim 1.

10. A compound as claimed in claim 9 wherein, independently or in any combination:
X is O; and
A is halophenyl or 2-pyridyl substituted with halo.

11. A compound as claimed in claim 10, wherein A is 3-halo phenyl or 6-halo pyridin-2-yl.

12. A compound as claimed in claim 11, wherein the halo substitutent is fluoro.

13. A compound as claimed in claim 1, wherein any substituents on the ring B is in a position other than that adjacent the atom which links the group B to the —NH—(CH₂)ₙ— moiety of the remainder of the molecule.

14. A compound as claimed in claim 1, wherein B is a fused 5,6- or 6,6-bicyclic aryl or heterobiaryl group, which may be partially saturated.

15. A compound as claimed in claim 14, wherein B is benzothiazole, benzimidazole, benzoxazole, naphthalene, quinoline, benzothiophene or phenyl substituted by two OR¹⁰ groups where the R¹⁰ groups together form an alkylene or alkenylene bridge having one to three carbon atoms.

16. A compound as claimed in claim 14, wherein the group B is a fused 5,6-bicyclic system joined to the remainder of the molecule via the 5-membered ring.

17. A compound as claimed in claim 16, wherein B is 2-benzothiazole, 2-benzimidazole or 2-benzoxazole.

18. A compound as claimed in claim 14, wherein the group B is a fused 5,6-bicyclic moiety joined to the remainder of the molecule via the 6-membered ring.

19. A compound as claimed in claim 18, wherein B is a 5-benzo[b]thiophen group.

20. A compound as claimed in claim 14, wherein B is benzodioxolyl, benzodioxinyl, dihydrobenzodioxinyl, 3-quinoline or naphthalene.

21. A compound as claimed in claim 14, wherein the group B is unsubstituted or is substituted with halogen, R¹⁰, —NR¹⁰R¹¹, —CONR¹⁰R¹¹, —NHCOR¹¹, —NR¹⁰SO₂R¹¹ CO₂R¹⁰ or OR¹⁰, where R¹⁰ and R¹¹ are each independently hydrogen, C₁₋₄ haloalkyl, C₁₋₄ alkyl, C₃₋₆ cycloalkyl, C₃₋₇ heterocyclyl or a 5- or 6-membered aromatic or heteroaromatic ring.

22. A compound as claimed in claim 21, wherein the group B is unsubstituted or is substituted with halogen, C₁₋₄ alkyl, C₁₋₄ haloalkyl, C₃₋₆ cycloalkyl, C₃₋₇ heterocyclyl or a 5- or 6-membered aromatic or heteroaromatic ring.

23. A compound as claimed in claim 22, wherein the group B is unsubstituted or substituted with halogen, methyl, ethyl, difluoromethyl, trifluoromethyl, cyclopropyl or pyridyl.

24. A compound as claimed in claim 1, wherein B is a monocyclic aryl or heteroaryl group having 5 or 6 ring atoms.

25. A compound as claimed in claim 24, wherein B is phenyl or pyridyl.

26. A compound as claimed in claim 25, wherein B is a 3-pyridyl group.

27. A compound as claimed in claim 25, wherein B is phenyl which is unsubstituted or mono-substituted at the 3- or the 4-position or disubstituted at the 3- and 4-positions.

28. A compound as claimed in claim 24, wherein B is unsubstituted or substituted with halo, CN, NO₂, R¹⁰, OR¹⁰, —CO₂R¹⁰, NR¹⁰, NR¹⁰R¹¹, NR¹⁰SO₂R¹¹, NR¹⁰COR¹¹, CONR¹⁰R¹¹, N(SO₂R¹⁰)₂, SO₂R¹⁰, SO₂NR¹⁰R¹¹ or O—(CH₂)_{q}-T, wherein R¹⁰, R¹¹, T and q are as defined in claim 1.

29. A compound as claimed in claim 28, wherein the group B is substituted with R¹⁰ or OR¹⁰ and R¹⁰ is hydrogen, C₁₋₆ alkyl, C₁₋₆ haloalkyl, a 5- or 6-membered cyclic or heterocyclic group or a 5- or 6-membered aryl or heteroaryl group, wherein the cyclic, heterocyclic, aryl or heteroaryl group are optionally substituted with a halo, C₁₋₄ alkyl, C₁₋₄ haloalkyl, O—C₁₋₄ alkyl, O—C₁₋₄ haloalkyl, CN, C(O)NH₂, C(O)NHCH₃, SO₂CH₃ or NO₂.

30. A compound as claimed in claim 29, wherein R¹⁰ is hydrogen (for) OR¹⁰), methyl, ethyl, isopropyl, n-propyl, n-butyl, isobutyl, t-butyl, difluoromethyl, trifluoromethyl, piperazinyl, morpholinyl, phenyl, pyridyl, triazolyl, imidazolyl, pyrazolyl, thiazolyl, oxadiazolyl and tetrazolyl, wherein the cyclic groups are optionally substituted as described in claim 1.

31. A compound as claimed in claim 28, wherein the group B is substituted with —CO₂R¹⁰, —COR¹⁰, and R¹⁰ is hydrogen, C₁₋₆ alkyl, C₁₋₆ haloalkyl, a 5- or 6-membered cyclic or heterocyclic group or a 5- or 6-membered aryl or heteroaryl group, wherein the cyclic, heterocyclic, aryl or heteroaryl groups are optionally substituted with a halo, C₁₋₄ alkyl or C₁₋₄ halo alkyl group and a pyridyl group may be present as an N-oxide.

32. A compound as claimed in claim 31, wherein R¹⁰ is hydrogen methyl, ethyl, morpholinyl, piperidinyl, phenyl or pyridyl wherein the cyclic groups may be substituted with a halo, C₁₋₄ alkyl or C₁₋₄ halo alkyl group, especially methyl or ethyl.

33. A compound as claimed in claim 28, wherein the group B is substituted with NR¹⁰R¹¹, NR¹⁰SO₂R¹¹, NR¹⁰COR¹¹, NR¹⁰CO₂R¹¹ or CONR¹⁰R¹¹, and R¹⁰ is H, or, in the case of CONR¹⁰R¹¹, R¹⁰ is C₁₋₄ alkyl or C₁₋₄ haloalkyl.

34. A compound as claimed in claim 28, wherein the group B is substituted with CONR¹⁰R¹¹, in which R¹⁰ and R¹¹ are both C₁₋₄ alkyl or C₁₋₄ haloalkyl.

35. A compound as claimed in claim 33, wherein R¹¹ is C₁₋₆ alkyl, C₁₋₆ haloalkyl, a 5- or 6-membered cyclic or heterocyclic group or a 5- or 6-membered aryl or heteroaryl group, wherein the cyclic, heterocyclic, aryl or heteroaryl groups may be further substituted with a halo, C₁₋₄ alkyl or C₁₋₄ halo alkyl group and a pyridyl group may be present as an N-oxide.

36. (canceled)

37. A compound as claimed in claim 28 or claim 29, wherein the group B is substituted with N(SO₂R¹⁰)₂, and each of the R¹⁰ groups is methyl or ethyl.

38. A compound as claimed in claim 28, wherein the group B is substituted with SOR¹⁰ or SO₂R¹⁰ and R¹⁰ is C₁₋₆ alkyl, C₃₋₆ cycloalkyl or C₃₋₆ heterocyclyl.

39. A compound as claimed in claim 38, wherein R¹⁰ is methyl, ethyl, isopropyl, cyclopentyl, morpholinyl or piperidinyl.

40. A compound as claimed in claim 28, wherein B is substituted with O—(CH₂)_{q}-T, and wherein q is 1 or 2 and the group T is a cycloalkyl, heterocyclyl, heteroaryl or aryl ring having 5 or 6 ring atoms.

41. A compound as claimed in claim 40, wherein T is phenyl, piperidinyl or morpholinyl.

42. A compound as claimed in claim 1 which is:
4-Benzoyl-piperazine-1-carbothioic acid (4-tert-butyl-phenyl)-amide (Compound 1);
4-Benzoyl-piperazine-1-carbothioic acid (4-dimethylamino-phenyl)-amide (Compound 2);
4-Benzoyl-piperazine-1-carbothioic acid (4-nitro-phenyl)-amide (Compound 3);
4-Benzoyl-piperazine-1-carbothioic acid (4-fluoro-phenyl)-amide (Compound 4);
4-Benzoyl-piperazine-1-carbothioic acid naphthalen-1-ylamide (Compound 5);
4-[(4-Benzoyl-piperazine-1-carbothioyl)-amino]benzoic acid methyl ester (Compound 6).
4-Benzoyl-piperazine-1-carbothioic acid p-tolylamide (Compound 7);
4-(4-Fluoro-benzoyl)-piperazine-1-carbothioic acid (4-tert-butyl-phenyl)-amide (Compound 8);
4-(4-Fluoro-benzoyl)-piperazine-1-carbothioic acid (4-phenoxy-phenyl)-amide (Compound 9);
4-(4-Fluoro-benzoyl)-piperazine-1-carbothioic acid (4-dimethylamino-phenyl)-amide (Compound 10);
4-{[4-(4-Fluoro-benzoyl)-piperazine-1-carbothioyl]-amino}-benzoic acid methyl ester (Compound 11);
4-(4-Fluoro-benzoyl)-piperazine-1-carbothioic acid (4-methoxy-phenyl)-amide (compound 12);
4-(4-Fluoro-benzoyl)-piperazine-1-carbothioic acid p-tolylamide (compound 13);
4-(Pyridine-3-carbonyl)-piperazine-1-carbothioic acid (4-dimethylamino-phenyl)-amide (Compound 14);
4-(6-fluoro-pyridine-2-carbonyl)-piperazine-1-carbothioic acid (6-morpholin-4-yl-pyridin-3-yl)-amide (Compound 15);
4-(6-Fluoro-pyridine-2-carbonyl)-piperazine-1-carbothioic acid (6-morpholin-4-yl-pyridin-3-yl)-amide (Compound 16);
4-(6-Fluoro-pyridine-2-carbonyl)-piperazine-1-carbothioic acid (4-phenoxy-phenyl)-amide (Compound 17);
4-(6-Fluoro-pyridine-2-carbonyl)-piperazine-1-carbothioic acid (4-difluoromethoxy-phenyl)-amide (Compound 18);
4-(6-Fluoro-pyridine-2-carbonyl)-piperazine-1-carbothioic acid (4-trifluoromethoxy-phenyl)-amide (Compound 19);
4-(6-Fluoro-pyridine-2-carbonyl)-piperazine-1-carbothioic acid (4-trifluoromethyl-phenyl)-amide (Compound 20);
4-(6-Fluoro-pyridine-2-carbonyl)-piperazine-1-carboxylic acid (4-trifluoromethoxy-phenyl)-amide (Compound 21);
4-(6-Fluoro-pyridine-2-carbonyl)-piperazine-1-carboxylic acid (4-trifluoromethyl-phenyl)-amide (Compound 22);
4-(3-Fluoro-benzoyl)-piperazine-1-carbothioic acid (4-trifluoromethyl-phenyl)-amide (Compound 23);
4-(3-Fluoro-benzoyl)-piperazine-1-carboxylic acid (2,6-dichloro-pyridin-4-yl)-amide (Compound 24);
4-(3-Fluoro-benzoyl)-piperazine-1-carboxylic acid (3-phenoxy-phenyl)-amide (Compound 25);
4-(3-Fluoro-benzoyl)-piperazine-1-carboxylic acid (4-phenoxy-phenyl)-amide (Compound 26);
4-(3-Fluoro-benzoyl)-piperazine-1-carboxylic acid benzo[b]thiophen-5-ylamide (Compound 27);
4-(3-Fluoro-benzoyl)-piperazine-1-carboxylic acid (4-thiophen-2-yl-phenyl)-amide (Compound 28);
4-(3-Fluoro-benzoyl)-piperazine-1-carboxylic acid [4-(2-methyl-thiazol-3-yl)-phenyl]-amide (Compound 29);
4-(3-Fluoro-benzoyl)-piperazine-1-carboxylic acid [3-(5-methyl-[1,2,4]oxadiazol-3-yl)-phenyl]-amide (Compound 30);
4-(3-Fluoro-benzoyl)-piperazine-1-carboxylic acid (3-morpholin-4-yl-phenyl)-amide (Compound 31);
4-(3-Fluoro-benzoyl)-piperazine-1-carboxylic acid (3-bromo-phenyl)-amide (Compound 32);
4-{[4-(3-Fluoro-benzoyl)-piperazine-1-carbonyl]-amino}-benzoic acid ethyl ester (Compound 33);
3-{[4-(3-Fluoro-benzoyl)-piperazine-1-carbonyl]amino}-benzoic acid methyl ester (Compound 34);
4-(3-Fluoro-benzoyl)-piperazine-1-carboxylic acid (3-cyano-phenyl)-amide (Compound 35);
4-(3-Fluoro-benzoyl)-piperazine-1-carboxylic acid (3-nitro-phenyl)-amide (Compound 36);
4-(3-Fluoro-benzoyl)-piperazine-1-carboxylic acid (4-nitro-phenyl)-amide (Compound 37);
4-(3-Fluoro-benzoyl)-piperazine-1-carboxylic acid (3-chloro-4-morpholin-4-yl-phenyl)-amide (Compound 38);
4-(3-Fluoro-benzoyl)-piperazine-1-carboxylic acid (5-chloro-benzooxazol-2-yl)-amide (Compound 39);
Preparation of 4-(6-Fluoro-pyridine-2-carbonyl)-piperazine-1-carbothioic acid (3-fluoro-4-morpholin-4-yl-phenyl)-amide (Compound 40);
4-(3-Fluoro-benzoyl)-piperazine-1-carboxylic acid (3-fluoro-4-morpholin-4-yl-phenyl)-amide (Compound 41);
4-(3-Fluoro-benzoyl)-piperazine-1-carboxylic acid (4-benzoyl-phenyl)-amide (Compound 42);
4-(3-Fluoro-benzoyl)-piperazine-1-carboxylic acid (4-bromo-3-pyridin-4-yl-phenyl)-amide (Compound 43);
4-(3-Fluoro-benzoyl)-piperazine-1-carboxylic acid [6-(pyridin-3-yloxy)-pyridin-3-yl]-amide (Compound 44);
4-(3-Fluoro-benzoyl)-piperazine-1-carboxylic acid quinolin-3-ylamide (Compound 45);
4-(2-Fluoro-4-{[4-(3-fluoro-benzoyl)-piperazine-1-carbonyl]-amino}-phenyl)-piperazine-1-carboxylic acid tert-butyl ester (Compound 46);
4-(2,5-Difluoro-benzoyl)-piperazine-1-carboxylic acid (3-fluoro-4-morpholin-4-yl-phenyl)-amide (Compound 47);
4-(4-{[4-(2,5-Difluoro-benzoyl)-piperazine-1-carbonyl]-amino}-2-fluoro-phenyl)-piperazine-1-carboxylic acid tert-butyl ester (Compound 48);
4-(2,5-Difluoro-benzoyl)-piperazine-1-carboxylic acid (4-nitro-phenyl)-amide (Compound 49);
4-(2-Fluoro-4-{[4-(3-fluoro-benzoyl)-piperazine-1-carbonyl]-amino}-phenyl)-piperazine-1-carboxylic acid isopropyl ester (Compound 50);
4-(2,5-Difluoro-benzoyl)-piperazine-1-carboxylic acid (3-fluoro-4-piperazin-1-yl-phenyl)-amide (Compound 51);
4-(3-fluorobenzoyl)-N-{3-fluoro-4-[4-(isopropylcarbamoyl)piperazin-1-yl]phenyl}piperazine-1-carboxamide (Compound 52);
N-{4-[4-(-butylcarbamoyl)piperazin-1-yl]-3-fluorophenyl}-4-(3-fluorobenzoyl)piperazine-1-carboxamide (Compound 53);
N-{4-[4-(cyclopentylcarbamoyl)piperazin-1-yl]-3-fluorophenyl}-4-(3-fluorobenzoyl)piperazine-1-carboxamide (Compound 54);
4-(2,5-difluorobenzoyl)-N-{3-fluoro-4-[4-(isopropylcarbamoyl)piperazin-1-yl]phenyl}piperazine-1-carboxamide (Compound 55);
4-(3-Fluoro-benzoyl)-piperazine-1-carboxylic acid {4-[4-(2,2-dimethyl-propionyl)-piperazin-1-yl]-3-fluoro-phenyl}-amide (Compound 56);
4-(3-Fluoro-benzoyl)-piperazine-1-carboxylic acid [3-fluoro-4-(4-isobutyryl-piperazin-1-yl)-phenyl]-amide (Compound 57);
4-(2,5-Difluoro-benzoyl)-piperazine-1-carboxylic acid [3-fluoro-4-(4-isobutyryl-piperazin-1-yl)-phenyl]-amide (Compound 58);
4-(2-Fluoro-4-{[4-(3-fluoro-benzoyl)-piperazine-1-carbonyl]-amino}-phenyl)-piperazine-1-carboxylic acid tetrahydro-furan-3-yl ester (Compound 59);
4-(3-Fluoro-benzoyl)-piperazine-1-carboxylic acid [3-fluoro-4-(4-methyl-piperazin-1-yl)-phenyl]-amide (Compound 60);
4-(3-Fluoro-benzoyl)-piperazine-1-carboxylic acid [4-(4-acetyl-piperazin-1-yl)-3-fluoro-phenyl]-amide (Compound 61);
4-(3-Fluoro-benzoyl)-piperazine-1-carboxylic acid (5-methyl-isoxazol-3-yl)-amide (Compound 62);
4-(3-Fluoro-benzoyl)-piperazine-1-carboxylic acid (6-chloro-pyridin-3-yl)-amide (Compound 63);
4-(3-Fluoro-benzoyl)-piperazine-1-carboxylic acid (1H-benzoimidazol-2-yl)-amide (Compound 64);
4-(3-Fluoro-benzoyl)-piperazine-1-carboxylic acid benzothiazol-2-ylamide (Compound 65);
4-(3-Fluoro-benzoyl)-piperazine-1-carboxylic acid (6-fluoro-benzothiazol-2-yl)-amide (Compound 66);
4-(3-Fluoro-benzoyl)-piperazine-1-carboxylic acid (6-trifluoromethoxy-benzothiazol-2-yl)-amide (Compound 67);
4-(3-Fluoro-benzoyl)-piperazine-1-carboxylic acid (6-bromo-benzothiazol-2-yl)-amide (Compound 68);
4-(3-Fluoro-benzoyl)-piperazine-1-carboxylic acid (4-phenyl-thiazol-2-yl)-amide (Compound 69);
4-(3-Fluoro-benzoyl)-piperazine-1-carboxylic acid (3-phenyl-[1,2,4]thiadiazol-5-yl)-amide (Compound 70);
4-(3-Fluoro-benzoyl)-piperazine-1-carboxylic acid (5-pyridin-4-yl-[1,3,4]thiadiazol-2-yl)-amide (Compound 71);
4-(3-Fluoro-benzoyl)-piperazine-1-carboxylic acid (5-phenyl-[1,3,4]oxadiazol-2-yl)-4-(3-Fluoro-benzoyl)-piperazine-1-carboxylic acid (4-pyridin-2-yl-thiazol-2-yl)-amide (Compound 73);
4-(3-Fluoro-benzoyl)-piperazine-1-carboxylic acid (4-pyridin-3-yl-thiazol-2-yl)-amide (Compound 74);
4-(3-Fluoro-benzoyl)-piperazine-1-carboxylic acid (4-pyridin-4-yl-thiazol-2-yl)-amide (Compound 75);
4-(3-Fluoro-benzoyl)-piperazine-1-carboxylic acid [3-(4-methyl-4H-[1,2,4]triazol-3-yl)-phenyl]-amide (Compound 76);
4-(3-Fluoro-benzoyl)-piperazine-1-carboxylic acid [3-(2-methyl-thiazol-4-yl)-phenyl]-amide (Compound 77);
4-(3-Fluoro-benzoyl)-piperazine-1-carboxylic acid [3-(2-methyl-pyrimidin-4-yl)-phenyl]-amide (Compound 78);
4-(3-Fluoro-benzoyl)-piperazine-1-carboxylic acid (5,6-dimethyl-benzothiazol-2-yl)-amide (Compound 79);
4-(3-Fluoro-benzoyl)-piperazine-1-carboxylic acid (6-methyl-benzothiazol-2-yl)-amide (Compound 80);
4-(3-Fluoro-benzoyl)-piperazine-1-carboxylic acid (6-methoxy-benzothiazol-2-yl)-amide (Compound 81);
4-(3-Fluoro-benzoyl)-piperazine-1-carboxylic acid (6-chloro-benzothiazol-2-yl)-amide (Compound 82);
2-{[4-(3-Fluoro-benzoyl)-piperazine-1-carbonyl]-amino}-benzothiazole-6-carboxylic acid ethyl ester (Compound 83);
4-(3-Fluoro-benzoyl)-piperazine-1-carboxylic acid (6-acetylamino-benzothiazol-2-yl)-amide (Compound 84);
4-(3-Fluoro-benzoyl)-piperazine-1-carboxylic acid (3-cyano-4-fluoro-phenyl)-amide (Compound 85);
4-(3-Fluoro-benzoyl)-piperazine-1-carboxylic acid (3-pyridin-4-yl-phenyl)-amide (Compound 86);
4-(3-Fluoro-benzoyl)-piperazine-1-carboxylic acid (3-pyridin-3-yl-phenyl)-amide (Compound 87);
4-(3-Fluoro-benzoyl)-piperazine-1-carboxylic acid (4-bromo-3-fluoro-phenyl)-amide (Compound 88);
4-(3-Fluoro-benzoyl)-piperazine-1-carboxylic acid [4-(1H-pyrazol-3-yl)-phenyl]-amide (Compound 89);
4-(3-Fluoro-benzoyl)-piperazine-1-carboxylic acid [4-(2-methyl-thiazol-4-yl)-phenyl]-amide (Compound 90);
4-(3-Fluoro-benzoyl)-piperazine-1-carboxylic acid [4-(morpholine-4-sulfonyl)-phenyl]-amide (Compound 91);
4-(3-Fluoro-benzoyl)-piperazine-1-carboxylic acid [3-(morpholine-4-sulfonyl)-phenyl]-amide (Compound 92);
4-(3-Fluoro-benzoyl)-piperazine-1-carboxylic acid (3-methylcarbamoyl-phenyl)-amide (Compound 93);
2-Fluoro-5-{[4-(3-fluoro-benzoyl)-piperazine-1-carbonyl]-amino}-benzoic acid methyl ester (Compound 94);
2-Fluoro-4-{[4-(3-fluoro-benzoyl)-piperazine-1-carbonyl]-amino}-benzoic acid methyl ester (Compound 95);
2-Fluoro-4-{[4-(3-fluoro-benzoyl)-piperazine-1-carbonyl]-amino}-benzoic acid (Compound 96);
4-(3-Fluoro-benzoyl)-piperazine-1-carboxylic acid [3-fluoro-4-(morpholine-4-carbonyl)-phenyl]-amide (Compound 97);
4-(3-Fluoro-benzoyl)-piperazine-1-carboxylic acid [3-(2-methyl-thiazol-4-yl)-phenyl]-amide (Compound 98);
4-(3-Fluoro-benzoyl)-piperazine-1-carboxylic acid [3-fluoro-4-(6-fluoro-pyridin-3-ylcarbamoyl)-phenyl]-amide (Compound 99);
4-{[4-(3-Fluoro-benzoyl)-piperazine-1-carbonyl]amino}-benzoic acid (Compound 100);
4-(3-Fluoro-benzoyl)-piperazine-1-carboxylic acid [4-(morpholine-4-carbonyl)-phenyl]-amide (Compound 101);
4-(3-Fluoro-benzoyl)-piperazine-1-carboxylic acid (4-diethylcarbamoyl-phenyl)-amide (Compound 102);
4-(3-Fluoro-benzoyl)-piperazine-1-carboxylic acid [4-(6-fluoro-pyridin-3-ylcarbamoyl)-phenyl]-amide (Compound 103);
4-(3-Fluoro-benzoyl)-piperazine-1-carboxylic acid (4-carbamoyl-3-fluoro-phenyl)-amide (Compound 104);
4-(3-Fluoro-benzoyl)-piperazine-1-carboxylic acid (4-carbamoyl-phenyl)-amide (Compound 105);
4-(3-Fluoro-benzoyl)-piperazine-1-carboxylic acid (3-amino-phenyl)-amide (Compound 106);
4-(3-Fluoro-benzoyl)-piperazine-1-carboxylic acid {3-[(pyridine-2-carbonyl)-amino]-phenyl}-amide (Compound 107);
4-(3-Fluoro-benzoyl)-piperazine-1-carboxylic acid {3-[(1-oxy-pyridine-2-carbonyl)-amino]-phenyl}-amide (Compound 108);
4-(3-Fluoro-benzoyl)-piperazine-1-carboxylic acid {3-[(1-oxy-pyridine-3-carbonyl)-amino]-phenyl}-amide (Compound 109);
4-(3-Fluoro-benzoyl)-piperazine-1-carboxylic acid {3-[(1-oxy-pyridine-4-carbonyl)-amino]-phenyl}-amide (Compound 110);
4-(3-Fluoro-benzoyl)-piperazine-1-carboxylic acid {3-[(6-fluoro-pyridine-3-carbonyl)-amino]-phenyl}-amide (Compound 111);
4-(3-Fluoro-benzoyl)-piperazine-1-carboxylic acid (4-amino-phenyl)-amide (Compound 112);
4-(2,5-Difluoro-benzoyl)-piperazine-1-carboxylic acid (4-amino-phenyl)-amide (Compound 113);
4-(3-Fluoro-benzoyl)-piperazine-1-carboxylic acid {4-[(pyridine-2-carbonyl)-amino]-phenyl}-amide (Compound 114);
4-(3-Fluoro-benzoyl)-piperazine-1-carboxylic acid {4-[(1-oxy-pyridine-2-carbonyl)-amino]-phenyl}-amide (Compound 115);
4-(3-Fluoro-benzoyl)-piperazine-1-carboxylic acid {4-[(1-oxy-pyridine-3-carbonyl)-amino]-phenyl}-amide (Compound 116);
4-(3-Fluoro-benzoyl)-piperazine-1-carboxylic acid {4-[(6-fluoro-pyridine-3-carbonyl)-amino]-phenyl}-amide (Compound 117);
4-(2,5-Difluoro-benzoyl)-piperazine-1-carboxylic acid {4-[(1-oxy-pyridine-2-carbonyl)-amino]-phenyl}-amide (Compound 118);
4-(2,5-Difluoro-benzoyl)-piperazine-1-carboxylic acid {4-[(1-oxy-pyridine-3-carbonyl)-amino]-phenyl}-amide (Compound 119);
4-(3-Fluoro-benzoyl)-piperazine-1-carboxylic acid [4-(4-fluoro-benzoylamino)-phenyl]-amide (Compound 120);
4-(3-Fluoro-benzoyl)-piperazine-1-carboxylic acid (4-isobutyrylamino-phenyl)-amide (Compound 121);
4-(3-Fluoro-benzoyl)-piperazine-1-carboxylic acid {4-[(pyridine-3-carbonyl)-amino]-phenyl}-amide (Compound 122);
4-(3-Fluoro-benzoyl)-piperazine-1-carboxylic acid {4-[(pyridine-4-carbonyl)-amino]-phenyl}-amide (Compound 123);
4-(3-Fluoro-benzoyl)-piperazine-1-carboxylic acid {4-[(tetrahydro-pyran-4-carbonyl)-amino]-phenyl}-amide (Compound 124);
4-(3-Fluoro-benzoyl)-piperazine-1-carboxylic acid (4-benzenesulfonylamino-phenyl)-amide (Compound 125);
4-(3-Fluoro-benzoyl)-piperazine-1-carboxylic acid (4-methanesulfonylamino-phenyl)-amide (Compound 126);
4-(3-Fluoro-benzoyl)-piperazine-1-carboxylic acid (4-cyclopropanesulfonylamino-phenyl)-amide (Compound 127);
4-(3-Fluoro-benzoyl)-piperazine-1-carboxylic acid [4-(4-fluoro-benzenesulfonylamino)-phenyl]-amide (Compound 128);
N-{4-[bis(methylsulfonyl)amino]phenyl}-4-(3-fluorobenzoyl)piperazine-1-carboxamide (Compound 129);
4-(3-Fluoro-benzoyl)-piperazine-1-carboxylic acid [3-(4-fluoro-benzenesulfonylamino)-phenyl]-amide (Compound 130);
4-(3-Fluoro-benzoyl)-piperazine-1-carboxylic acid (3-cyclopropanesulfonylamino-phenyl)-amide (Compound 131);
4-(2,5-Difluoro-benzoyl)-piperazine-1-carboxylic acid (4-cyclopropanesulfonylamino-phenyl)-amide (Compound 132);
4-(2,5-Difluoro-benzoyl)-piperazine-1-carboxylic acid (4-methanesulfonylamino-phenyl)-amide (Compound 133);
4-(3-Fluoro-benzoyl)-piperazine-1-carboxylic acid (3-fluoro-4-nitro-phenyl)-amide (Compound 134);
4-(3-Fluoro-benzoyl)-piperazine-1-carboxylic acid [4-(1H-tetrazol-5-yl)-phenyl]amide (Compound 135);
4-(3-Fluoro-benzoyl)-piperazine-1-carboxylic acid (6-amino-benzothiazol-2-yl)-amide (Compound 136);
4-(3-Fluoro-benzoyl)-piperazine-1-carboxylic acid (6-cyclopropanesulfonylamino-benzothiazol-2-yl)-amide (Compound 137);
2-{[4-(3-Fluoro-benzoyl)-piperazine-1-carbonyl]-amino}-benzothiazole-6-carboxylic acid (Compound 138);
4-(3-Fluoro-benzoyl)-piperazine-1-carboxylic acid [6-(morpholine-4-carbonyl)-benzothiazol-2-yl]-amide (Compound 139);
4-(3-Fluoro-benzoyl)-piperazine-1-carboxylic acid {6-[(pyridine-2-carbonyl)-amino]-benzothiazol-2-yl}-amide (Compound 140);
2-{[4-(3-Fluoro-benzoyl)-piperazine-1-carbonyl]-amino}-benzothiazole-6-carboxylic acid amide (Compound 141);
4-(3-Fluoro-benzoyl)-piperazine-1-carboxylic acid [4-(3-isopropyl-[1,2,4]oxadiazol-5-yl)-phenyl]-amide (Compound 142);
4-(3-Fluoro-benzoyl)-piperazine-1-carboxylic acid (4-pyrazol-1-yl-phenyl)-amide (Compound 143);
4-(3-Fluoro-benzoyl)-piperazine-1-carboxylic acid (4-imidazol-1-yl-phenyl)-amide (Compound 144);
4-(3-Fluoro-benzoyl)-piperazine-1-carboxylic acid (4-[1,2,4]triazol-1-yl-phenyl)-amide (Compound 145);
4-(3-Fluoro-benzoyl)-piperazine-1-carboxylic acid [3-fluoro-4-(4-pyridin-2-yl-piperazin-1-yl)-phenyl]-amide (Compound 146);
4-(3-Fluoro-benzoyl)-piperazine-1-carboxylic acid (3-fluoro-4-pyridin-3-yl-phenyl)-amide (Compound 147);
4-(2,5-Difluoro-benzoyl)-piperazine-1-carboxylic acid (4-trifluoromethyl-phenyl)-amide (Compound 148);
4-(3-Fluoro-benzoyl)-piperazine-1-carboxylic acid (4-trifluoromethyl-phenyl)-amide (Compound 149);
4-(2,5-Difluoro-benzoyl)-piperazine-1-carbothioic acid (6-morpholin-4-yl-pyridin-3-yl)-amide (Compound 150);
4-(3-Fluoro-benzoyl)-piperazine-1-carbothioic acid (6-morpholin-4-yl-pyridin-3-yl)-amide (Compound 151);
4-(2-Methyl-benzoyl)-piperazine-1-carbothioic acid (6-morpholin-4-yl-pyridin-3-yl)-amide (Compound 152);
4-(3-Nitro-benzoyl)-piperazine-1-carbothioic acid (6-morpholin-4-yl-pyridin-3-yl)-amide (Compound 153);
4-(3-Fluoro-benzoyl)-piperazine-1-carboxylic acid (6-morpholin-4-yl-pyridin-3-yl)-amide (Compound 154);
4-(3-Fluoro-benzoyl)-piperazine-1-carboxylic acid (4-fluoro-phenyl)-amide (Compound 155);
4-(2-Methyl-benzoyl)-piperazine-1-carboxylic acid (4-fluoro-phenyl)-amide (Compound 156);
4-(3-Carbamoyl-benzoyl)-piperazine-1-carboxylic acid (4-fluoro-phenyl)-amide (Compound 157);
4-(3-Fluoro-benzoyl)-piperazine-1-carboxylic acid (3-carbamoyl-phenyl)-amide (Compound 158);
4-(3-Fluoro-benzoyl)-piperazine-1-carboxylic acid [3-(2H-[1,2,4]triazol-3-yl)-phenyl]-amide (Compound 159);
4-(3-Fluoro-benzoyl)-piperazine-1-carboxylic acid (3-carbamoyl-4-fluoro-phenyl)-amide (Compound 160);
4-(3-Fluoro-benzoyl)-piperazine-1-carboxylic acid [4-fluoro-3-(2H-[1,2,4]triazol-3-yl)-phenyl]-amide (Compound 161);
4-(3-Fluoro-benzoyl)-piperazine-1-carboxylic acid [3-(3-isopropyl-[1,2,4]oxadiazol-5-yl)-phenyl]-amide (Compound 162);
4-(3-Fluoro-benzoyl)-piperazine-1-carboxylic acid [4-fluoro-3-(3-isopropyl-[1,2,4]oxadiazol-5-yl)-phenyl]-amide (Compound 163);
4-Benzoyl-piperazine-1-carboxylic acid (4-trifluoromethyl-phenyl)-amide (Compound 164);
4-(3-Chloro-benzoyl)-piperazine-1-carboxylic acid (4-trifluoromethyl-phenyl)-amide (Compound 165);
4-(3-Fluoro-benzoyl)-piperazine-1-carboxylic acid (3-phenylamino-phenyl)-amide (Compound 166);
4-(3-Fluoro-benzoyl)-piperazine-1-carboxylic acid [3-(4-fluoro-phenylamino)-phenyl]-amide (Compound 167);
or a pharmacologically acceptable salt, hydrate, solvate, complex prodrug thereof.

43. A process for preparing a compound as claimed in claim 1, the process comprising:
a) reacting a compound of general formula (II). 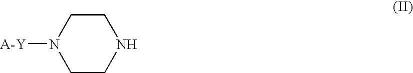
wherein A and Y are as defined in claim 1 with a compound of general formula (III):
B—(CH₂)ₙ—N═C═X (III)
wherein B and X are as defined in claim 1; or
b) reacting a compound of general formula (XIX) with a compound of general formula (IX):
A-C(O)Cl (IX)
wherein A is as defined in claim 1;
to give a compound of general formula (I) wherein Y is C═O; or
d) reacting a compound of general formula (XIX) with a compound of general formula (X):
A-C(O)OH (X)
wherein A is as defined in claim 1;
to give a compound of general formula (I) wherein Y is C═O; or
e) reacting a compound of general formula (XII):
B—(CH₂)ₙ—NH₂ (XII)
wherein B and n are as defined in claim 1;
with a chloroformate followed by a compound of general formula (II) as defined above; or
f) reacting a compound of general formula (XXX):
B—C(═O)OH (XXX)
wherein B is as defined in claim 1;
with a stable azide-transfer agent in the presence of an amine, to form the corresponding acyl azide intermediate, followed by reaction with a compound of general formula (II); or
g) converting a compound as claimed in claim 1 to another compound as claimed in claim 1.

44. A compound as claimed in claim 1 or a pharmacologically acceptable salt, hydrate, solvate, complex prodrug thereof for use in medicine.

45. A compound as claimed in claim 1 or a pharmacologically acceptable salt, hydrate, solvate, complex prodrug thereof for use in the treatment or prevention of metabolic disorders, inflammatory conditions, allergic conditions, fever, pain including allodynia and nociception, eating disorders, cachexia, brain injuries, cancer of the genitals, sleep apnoea, cardiovascular disease, flush effect associated with nicotinic acid and related compounds or for the promotion of wound healing.

46. A method of treating a patient in need thereof comprising administering a compound as claimed in claim 1 or a pharmacologically acceptable salt, hydrate, solvate, complex prodrug thereof in an amount effective to treat metabolic disorders, inflammatory conditions, allergic conditions, fever, pain including allodynia and nociception, eating disorders, cachexia, brain injuries, cancer of the genitals, sleep apnoea, cardiovascular disease, flush effect associated with nicotinic acid and related compounds or for the promotion of wound healing in said patient.

47. A compound or a pharmacologically acceptable salt, hydrate, solvate, complex prodrug thereof as claimed in claim 41, wherein the metabolic disorder is metabolic syndrome, a disorder of the lipid or carbohydrate metabolic system, for example, obesity, inflammation associated with obesity, impaired glucose tolerance, diabetes mellitus (particularly diabetes mellitus type I, diabetes mellitus type II and latent autoimmune diabetes in adults) and complications thereof, and lipid disorders and complications thereof.

48. A compound or the use as claimed in claim 43, wherein the complications associated with diabetes are diabetic gangrene, diabetic arthropathy, diabetic osteopenia, diabetic glomerosclerosis, diabetic nephropathy, diabetic dermopathy, diabetic neuropathy, diabetic cataract and diabetic retinopathy, diabetic maculopathy, diabetic feet syndrome, diabetic coma with or without ketoacidosis, diabetic hyperosmolar coma, hypoglycemic coma, hyperglycemic coma, diabetic acidosis, diabetic ketoacidosis, intracapillary glomerulonephrosis, Kimmelstiel-Wilson syndrome, diabetic amyotrophy, diabetic autonomic neuropathy, diabetic mononeuropathy, diabetic polyneuropathy, diabetic angiopathies, diabetic peripheral angiopathy, diabetic ulcer, diabetic arthropathy and obesity in diabetes.

49. A compound as claimed in claim 43, wherein the complications associated with lipid disorders include hypercholesterolemia, familial hypercholesterolemia, Fredrickson's hyperlipoproteinemia, hyperbetalipoproteinemia, hyperlipidemia, low-density-lipoprotein-type [LDL] hyperlipoproteinemia, pure hyperglyceridemia, endogenous hyperglyceridemia, isolated hypercholesterolemia, isolated hypertroglyceridemia, cardiovascular diseases such as hypertension, ischemia, varicose veins, retinal vein occlusion, atherosclerosis, stroke, thrombosis, angina pectoris, myocardial infarction, stenocardia, pulmonary hypertension, congestive heart failure, glomerulopaty, tubulointestitial disorders, renal failure, angiostenosis, or cerebrovascular disorders, such as cerebral apoplexy.

50. A compound or a pharmacologically acceptable salt, hydrate, solvate, complex prodrug thereof as claimed in claim 41, wherein the inflammatory condition is granuloma, systemic lupus erythematosus, inflammatory bowel disease, Crohn's disease, ulcerative colitis, multiple sclerosis and other demylelinating diseases, osteoarthritis, rheumatoid arthritis, psoriatic arthritis, systemic vasculitis, amyotrophic lateral sclerosis (ALS or Lou Gehrig's disease), hypothyroidism, chronic obstructive pulmonary disease (COPD), asthma, psoriasis, wound healing, and brain injury.

51. A compound or a pharmacologically acceptable salt, hydrate, solvate, complex prodrug thereof as claimed in claim 41, wherein the allergic condition is anaphylaxis, allergic rhinitis (hay fever) and mastocytosis.

52. A compound as claimed in claim 1 or a pharmacologically acceptable salt, hydrate, solvate, complex prodrug thereof for use in the treatment of diabetes and wherein B is a monocyclic ring system substituted with R¹⁰ and the R¹⁰ group is C₁₋₆ alkyl, C₁₋₆ haloalkyl, 5- or 6-membered cyclic or heterocyclic groups or 5- or 6-membered aryl or heteroaryl groups, wherein the cyclic, heterocyclic, aryl or heteroaryl groups may be further substituted with a halo, C₁₋₄ alkyl, C₁₋₄ haloalkyl or C₃₋₆ cycloalkyl, and a pyridyl group may be present as an N-oxide.

53. A method of treating a patient with diabetes comprising administering a compound as claimed in claim 1 or a pharmacologically acceptable salt, hydrate, solvate, complex prodrug thereof, wherein B is a monocyclic ring system substituted with R¹⁰ and the R¹⁰ group is C₁₋₆ alkyl, C₁₋₆ haloalkyl, 5- or 6-membered cyclic or heterocyclic groups or 5- or 6-membered aryl or heteroaryl groups, wherein the cyclic, heterocyclic, aryl or heteroaryl groups may be further substituted with a halo, C₁₋₄ alkyl, C₁₋₄ haloalkyl or C₃₋₆ cycloalkyl, and a pyridyl group may be present as an N-oxide.

54. A compound or a pharmacologically acceptable salt, hydrate, solvate, complex prodrug thereof as claimed in claim 52, wherein R¹⁰ is methyl, ethyl, isopropyl, n-propyl, n-butyl, isobutyl, t-butyl, trifluoromethyl, morpholinyl, phenyl, pyridyl, triazolyl, imidazolyl, pyrazolyl, thiazolyl, oxadiazolyl and tetrazolyl, wherein the cyclic groups may be substituted as the complications associated with diabetes are diabetic gangrene, diabetic arthropathy, diabetic osteopenia, diabetic glomerosclerosis, diabetic nephropathy, diabetic dermopathy, diabetic neuropathy, diabetic cataract and diabetic retinopathy, diabetic maculopathy, diabetic feet syndrome, diabetic coma with or without ketoacidosis, diabetic hyperosmolar coma, hypoglycemic coma, hyperglycemic coma, diabetic acidosis, diabetic ketoacidosis, intracapillary glomerulonephrosis, Kimmelstiel-Wilson syndrome, diabetic amyotrophy, diabetic autonomic neuropathy, diabetic mononeuropathy, diabetic polyneuropathy, diabetic angiopathies, diabetic peripheral angiopathy, diabetic ulcer, diabetic arthropathy and obesity in diabetes, and the complications associated with lipid disorders include hypercholesterolemia, familial hypercholesterolemia, Fredrickson's hyperlipoproteinemia, hyperbetalipoproteinemia, hyperlipidemia, low-density-lipoprotein-type [LDL] hyperlipoproteinemia, pure hyperglyceridemia, endogenous hyperglyceridemia, isolated hypercholesterolemia, isolated hypertroglyceridemia, cardiovascular diseases such as hypertension, ischemia, varicose veins, retinal vein occlusion, atherosclerosis, stroke, thrombosis, angina pectoris, myocardial infarction, stenocardia, pulmonary hypertension, congestive heart failure, glomerulopaty, tubulointestitial disorders, renal failure, angiostenosis, or cerebrovascular disorders, such as cerebral apoplexy.

55. A pharmaceutical composition comprising a compound as claimed in claim 1 or a pharmacologically acceptable salt, hydrate, solvate, complex prodrug thereof together with a pharmaceutical excipiept or carrier.

56. A pharmaceutical composition as claimed in claim 47 which is adapted to be administered orally, parenteraly, such as subcutaneously, intravenously, intramuscularly, intraperitoneally, intrathecally, transdermally, transmucosally, subdurally, locally or topically via iontopheresis, sublingually, by inhalation spray, aerosol, or rectally.

57. A pharmaceutical composition as claimed in claim 55 further comprising an additional therapeutic agent.

58. A pharmaceutical composition as claimed in claim 57, wherein the additional therapeutic agent is selected from antidiabetics like insulin, long and short acting insulin analogues, sulfonylureas and other antidiabetics derived from thiazolidindiones, lipid lowering agents such as statines, fibrates, ion exchange resins, nicotinic acid and derivatives thereof, or HMG-CoA reductase inhibitors, cardiovascular therapeutics such as nitrates, antihypertensiva such as β-blockers, ACE inhibitors, Ca-channel blockers, angiotensin II receptor antagonists, diuretics, thrombocyte aggregation inhibitors, or antineoplastic agents such as alkaloids, alkylating agents, antibiotics or antimetabolites.

59. A pharmaceutical composition as claimed in claim 58, wherein the additional therapeutic agent is selected from human NPH insulin, human lente or ultralente insulin, insulin Lispro, insulin Asptart, or insulin Glargine, nicotinic acid atenolol, bisoprolol, metoprolol, esmolol, celiprolol, talinolol, oxprenolol, pindolol, propanolol, bupropanolol, penbutolol, mepindolol, sotalol, certeolol, nadolol, carvedilol, nifedipin, nitrendipin, amlodipin, nicardipin, nisoldipin, diltiazem, enalapril, verapamil, gallopamil, quinapril, captopril, lisinopril, benazepril, ramipril, peridopril, fosinopril, trandolapril, irbesatan, losartan, valsartan, telmisartan, eprosartan, olmesartan, hydrochlorothiazide, piretanid, chlorotalidone, mefruside, furosemide, bendroflumethiazid, triamterene, dehydralazine, acetylsalicylic acid, tirofiban-HCl, dipyramidol, triclopidin, iloprost-trometanol, eptifibatide, clopidogrel, piratecam, abciximab, trapidil, simvastatine, bezafibrate, fenofibrate, gemfibrozil, etofyllin, clofibrate, etofibrate, fluvastatine, lovastatine, pravastatin, colestyramide, colestipol-HCl, xantinol nicotinat, inositol nicotinat, acipimox, nebivolol, glycerolnitrate, isosorbide mononitrate, isosorbide dinitrate, pentaerythrityl tetranitrate, indapamide, cilazepril, urapidil, eprosartan, nilvadipin, metoprolol, doxazosin, molsidormin, moxaverin, acebutolol, prazosine, trapidil, clonidine, vinca alkaloids and analogues such as vinblastin, vincristin, vindesin, vinorelbin, podophyllotoxine derivatives, etoposid, teniposid, alkylating agents, nitroso ureas, N-lost analogues, cycloplonphamid, estamustin, melphalan, ifosfamid, mitoxantron, idarubicin, doxorubicin, bleomycin, mitomycin, dactinomycin, daptomycin, antimetabolites such as cytarabin, fluorouracil, fluoroarabin, gemcitabin, tioguanin, capecitabin, combinations such as adriamydin/daunorubicin, cytosine arabinosid/cytarabine, 4-HC, or other phosphamides.

60. A product comprising a compound as claimed in claim 1 or a pharmacologically acceptable salt, hydrate, solvate, complex prodrug thereof and one or more of the agents listed in claim 54 as a combined preparation for simultaneous, separate or sequential use in the treatment or prevention of of metabolic disorders, inflammatory conditions, allergic conditions, fever, pain including allodynia and nociception, eating disorders, cachexia, brain injuries, cancer of the genitals, sleep apnoea, cardiovascular disease, flush effect associated with nicotinic acid and related compounds or for the promotion of wound healing.

61. A method of treating a patient in need thereof comprising administering a compound of claim 47 and co-administering an additional therapeutic agent, wherein said additional therapeutic agent is selected from group consisting of antidiabetics, lipid lowering agents, HMG-CoA reductase inhibitors, cardiovascular therapeutics, antihypertensives, ACE inhibitors, Ca²⁺-channel blockers, angiotensin II receptor antagonists, diuretics, thrombocyte aggregation inhibitors, antineoplastic agents, alkylating agents, antibiotics, an antimetabolites

62. A compound as claimed in claim 37, wherein R¹¹ is methyl, ethyl, isopropyl, n-propyl, n-butyl, isobutyl, t-butyl, trifluoromethyl, cyclopropyl, tetrahydropyranyl, phenyl or pyridyl wherein the cyclic, aryl or heteroaryl groups may be substituted with a halo, C₁₋₄ alkyl or C₁₋₄ halo alkyl group, especially methyl or ethyl and a pyridyl group may be present as its N-oxide.
